(19) United States
(12) Patent Application Publication
(10) Pub. No.: US 2023/0138177 A1
(43) Pub. Date: May 4, 2023

(54) PROLINE HYDROXYLATION PRIMES PROTEIN KINASES FOR AUTOPHOSPHORYLATION AND ACTIVATION

(71) Applicant: The Trustees of Columbia University in the City of New York, New York, NY (US)

(72) Inventors: Antonio IAVARONE, Sunny Isles Beach, FL (US); Anna LASORELLA, New York, NY (US)

(21) Appl. No.: 18/148,841

(22) Filed: Dec. 30, 2022

Related U.S. Application Data
(63) Continuation of application No. PCT/US2021/038691, filed on Jun. 23, 2021.
(60) Provisional application No. 63/047,448, filed on Jul. 2, 2020.

(51) Int. Cl. C12Q 1/48; C12N 9/02

(52) U.S. Cl. CPC C12Q 1/485; C12N 9/0071; C12Y 114/11029; C12Y 207/12001; G01N 2333/912; G01N 2440/24

(57) ABSTRACT

The subject matter described herein relates to a method of regulating protein kinase activity by allosteric modifications such as prolyl hydroxylation.

This application is a continuation of International Patent Application No. PCT/US2021/038691, filed on Jun. 23, 2021, which claims the benefit of and priority to U.S. Provisional Patent Application No. 63/047,448, filed on Jul. 2, 2020, the entire contents of each of these applications are hereby incorporated by reference in their entirety.

### GOVERNMENT SUPPORT

The work described herein was supported in whole, or in part, by National Institutes Health Grant Nos. 5R01CA239721 and R01CA178546. Thus, the United States Government has certain rights to the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

A kinase is an enzyme that can transfer a phosphate group from ATP to specific residues of proteins inside a cell. These residues can be tyrosine, serine, or threonine. Phosphorylation functions as an “on” or “off” switch in many cellular functions.

### SUMMARY OF THE INVENTION

In certain aspects, the invention provides a method of modulating kinase activity, the method comprising modulating the prolyl hydroxylation status of said kinase.

In some embodiments, the kinase is a CMGC kinase. In some embodiments, the CMGC kinase is a dual specificity tyrosine-phosphorylation-regulated kinase (DYRK). In some embodiments, the DYRK kinase is DYRK1. In some embodiments, the DYRK1 kinase is DYRK1A. In some embodiments, the DYRK1 kinase is DYRK1B. In some embodiments, the DYRK kinase is DYRK2. In some embodiments, the DYRK kinase is DYRK3. In some embodiments, the DYRK kinase is DYRK4. In some embodiments, the CMGC kinase is a MAPK kinase. In some embodiments, the CMGC kinase is a GSK3 kinase. In some embodiments, the CMGC kinase is a HIPK kinase. In some embodiments, the CMGC kinase is a CDK kinase.

In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of the kinase. In some embodiments, DYRK1A is hydroxylated on proline 380 of the amino acid sequence. In some embodiments, DYRK1B is hydroxylated on proline 332 of the amino acid sequence.

In some embodiments, DYRK1B prolyl hydroxylation results in phosphorylation of ID2. In some embodiments, DYRK1B prolyl hydroxylation releases VHL ubiquitin ligase from ID2. In some embodiments, the modulation comprises increasing kinase activity. In some embodiments, the modulation comprises decreasing kinase activity.

In some embodiments, prolyl hydroxylation of the kinase increases kinase activity. In some embodiments, prolyl hydroxylation results in kinase autophosphorylation. In some embodiments, prolyl hydroxylation of the kinase results in phosphorylation of downstream targets of the kinase.

In some embodiments, prolyl hydroxylation of the kinase results in tumor growth suppression. In some embodiments, prolyl hydroxylation of the kinase results in glioma growth suppression.

In some embodiments, the modulator is a small molecule. In some embodiments, the modulator acts on a PHD hydroxylase to alter its activity toward the kinase. In some embodiments, the modulator is CoCl₂. In some embodiments, the modulator is dimethyloxalylglycine (DMOG). In some embodiments, the modulation comprises genetic engineering of the kinase or of a hydroxylase acting on the kinase.

In certain aspects, the invention provides a method of activating DYRK1A kinase, the method comprising promoting prolyl hydroxylation of the kinase. In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of DYRK1A. In some embodiments, DYRK1A is hydroxylated on proline residue 380 of the amino acid sequence. In some embodiments, prolyl hydroxylation of DYRK1A results in DYRK1A autophosphorylation.

In some embodiments, prolyl hydroxylation of DYRK1A results in phosphorylation of DYRK1A downstream targets. In some embodiments, prolyl hydroxylation of DYRK1A results in tumor growth suppression. In some embodiments, In some embodiments, prolyl hydroxylation of DYRK1A results in glioma growth suppression.

In some embodiments, the method comprises a small molecule activator. In some embodiments, the method comprises an activator acting on a PHD hydroxylase to alter its activity toward DYRK1A. In some embodiments, the method comprises genetic engineering of DYRK1A or genetic engineering of a hydroxylase acting on DYRK1A.

In certain aspects, the invention provides a method of activating DYRK1B kinase, the method comprising promoting prolyl hydroxylation of the kinase.

In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of DYRK1B. In some embodiments, DYRK1B is hydroxylated on proline residue 332 of the amino acid sequence. In some embodiments, prolyl hydroxylation of DYRK1B results in DYRK1B autophosphorylation. In some embodiments, DYRK1B prolyl hydroxylation results in phosphorylation of ID2. In some embodiments, prolyl hydroxylation of DYRK1B results in phosphorylation of downstream targets of the kinase. In some embodiments, DYRK1B prolyl hydroxylation releases VHL ubiquitin ligase from ID2.

In some embodiments, prolyl hydroxylation of DYRK1B results in tumor growth suppression. In some embodiments, prolyl hydroxylation of DYRK1B results in glioma growth suppression.

In some embodiments, the method comprises a small molecule activator. In some embodiments, the method comprises an activator acting on a PHD hydroxylase to alter its activity toward DYRK1B. In some embodiments, the method comprises genetic engineering of DYRK1B or genetic engineering of a hydroxylase acting on DYRK1B.

In certain aspects, the invention provides a method of treating cancer in a subject in need thereof, the method comprising administering to the subject a therapeutically effective amount of a composition capable of modulating kinase.

In some embodiments, the kinase is a CMGC kinase. In some embodiments, the CMGC kinase is a dual specificity tyrosine-phosphorylation-regulated kinase (DYRK). In some embodiments, the DYRK kinase is DYRK1. In some embodiments, the DYRK1 kinase is DYRK1A. In some embodiments, the DYRK1 kinase is DYRK1B. In some embodiments, the DYRK kinase is DYRK2. In some embodiments, the DYRK kinase is DYRK3. In some embodiments, the DYRK kinase is DYRK4. In some embodiments, the CMGC kinase is a MAPK kinase. In some embodiments, the CMGC kinase is a GSK3 kinase. In some embodiments, the CMGC kinase is a HIPK kinase. In some embodiments, the CMGC kinase is a CDK kinase.

In some embodiments, the composition promotes prolyl hydroxylation of said kinase. In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of the kinase. In some embodiments, DYRK1A is hydroxylated on proline 380 of the amino acid sequence. In some embodiments, DYRK1B is hydroxylated on proline 332 of the amino acid sequence. In some embodiments, DYRK1B prolyl hydroxylation results in phosphorylation of ID2. In some embodiments, DYRK1B prolyl hydroxylation releases VHL ubiquitin ligase from ID2.

In some embodiments, the modulation comprises increasing kinase activity. In some embodiments, the modulation comprises decreasing kinase activity. In some embodiments, prolyl hydroxylation of the kinase increases kinase activity. In some embodiments, prolyl hydroxylation of the kinase results in the kinase autophosphorylation.

In some embodiments, prolyl hydroxylation of the kinase results in phosphorylation of downstream targets of the kinase. In some embodiments, prolyl hydroxylation of the kinase results in cancer suppression. In some embodiments, the cancer is a glioma. In some embodiments, the cancer is a breast cancer. In some embodiments the cancer is a prostate cancer. In some embodiments, the cancer is a lung cancer. In some embodiments, the cancer is a bladder cancer. In some embodiments, the cancer is a colorectal cancer. In some embodiments, the cancer is the cancer is melanoma. In some embodiments, the cancer is a kidney cancer. In some embodiments, the modulator is a small molecule.

In some embodiments, the modulator acts on a PHD hydroxylase to alter its activity toward the kinase. In some embodiments, the modulator is CoCl₂. In some embodiments, the modulator is dimethyloxalylglycine (DMOG). In some embodiments, the modulation comprises genetic engineering of the kinase or genetic engineering of a hydroxylase acting on the kinase.

In certain aspects, the invention provides a method for decreasing tumor size in a subject in need thereof, the method comprising administering to the subject a therapeutically effective amount of a composition capable of modulating kinase activity.

In some embodiments, the kinase is a CMGC kinase. In some embodiments, the CMGC kinase is a dual specificity tyrosine-phosphorylation-regulated kinase (DYRK). In some embodiments, the DYRK kinase is DYRK1. In some embodiments, the DYRK1 kinase is DYRK1A. In some embodiments, the DYRK1 kinase is DYRK1B. In some embodiments, the DYRK kinase is DYRK2. In some embodiments, the DYRK kinase is DYRK3. In some embodiments, the DYRK kinase is DYRK4. In some embodiments, the CMGC kinase is a MAPK kinase. In some embodiments, the CMGC kinase is a GSK3 kinase. In some embodiments, the CMGC kinase is a HIPK kinase. In some embodiments, the CMGC kinase is a CDK kinase.

In some embodiments, the composition promotes prolyl hydroxylation of said kinase. In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of the kinase. In some embodiments, DYRK1A is hydroxylated on proline 380 of the amino acid sequence. In some embodiments, DYRK1B is hydroxylated on proline 332 of the amino acid sequence. In some embodiments, DYRK1B prolyl hydroxylation results in phosphorylation of ID2. In some embodiments, DYRK1B prolyl hydroxylation releases VHL ubiquitin ligase from ID2.

In some embodiments, the modulation comprises increasing kinase activity. In some embodiments, the modulation comprises decreasing kinase activity. In some embodiments, prolyl hydroxylation of the kinase increases kinase activity. In some embodiments, prolyl hydroxylation of the kinase results in the kinase autophosphorylation. In some embodiments, prolyl hydroxylation of the kinase results in phosphorylation of downstream targets of the kinase.

In some embodiments, prolyl hydroxylation of the kinase results in tumor growth suppression. In some embodiments, the tumor is a glioma tumor. In some embodiments, the tumor is a tumor in the breast tissue. In some embodiments, the tumor is a tumor in the prostate tissue. In some embodiments, the tumor is a tumor in the lung tissue. In some embodiments, the tumor is a tumor in the bladder tissue. In some embodiments, the tumor is a tumor in the colorectal tissue. In some embodiments, the tumor is melanoma tumor. In some embodiments, the tumor is a tumor in the kidney.

In some embodiments, the modulator is a small molecule. In some embodiments, the modulator acts on a PHD hydroxylase to alter the activity of the hydroxylase toward the kinase. In some embodiments, the modulator is CoCl₂. In some embodiments, the modulator is dimethyloxalylglycine (DMOG). In some embodiments, the modulation comprises genetic engineering of the kinase or genetic engineering of a hydroxylase acting on the kinase.

### BRIEF DESCRIPTION OF THE FIGURES

The patent or application file contains at least one drawing in color. To conform to the requirements for PCT patent applications, many of the figures presented herein are black and white representations of images originally created in color.

FIGS. 1A-G show that proline 332 of DYRK1B is hydroxylated by PHD1. (FIG. 1A) Recombinant GSTDYRK1B was incubated in the presence or absence of PHD1 and immunoprecipitated using hydroxyl proline antibody. Immunoprecipitated proteins were processed by immunoblot using DYRK1B antibody. Total proteins in the in vitro reaction were included as control. (FIG. 1B) Hydroxyl proline or phospho-tyrosine antibody were used in immunoprecipitation assay in U87 cells. Proteins were detected by western blot using DYRK1A or DYRK1B antibodies. (FIG. 1C) U87 glioma cells were co-transfected with FLAGDYRK1B and MYC-PHD1 or the empty vector. Prolyl hydroxylated FLAG-DYRK1B was captured from sequential immunoprecipitations with hydroxyl-proline followed by reprecipitation with FLAG antibodies. Shown are liquid chromatography-tandem mass spectrometry (LC-MS/MS) data corresponding to DYRK1B peptides hydroxylated at Pro332 (lower panel) or non-hydroxylated (upper panel, from single FLAG immunoprecipitates in cells lacking PHD1). The sequence of the identified peptide is: IVEVLGIP#PAAMLDQAPK. A +16-Da shift is observed in the y-ion series at the y¹¹ ion, corresponding to fragments containing P332. Peak heights are relative abundances of the corresponding fragment ions. In green are the identified N-terminus containing ions, b ions; in black are C-terminus containing ions, y ions. Amino acid sequence alignment of DYRK1B and DYRK1A comprising DYRK1B peptide for which LC-MS/MS data are presented (bold). Hydroxylated P332 in DYRK1B and the conserved P380 in DYRK1A are in red. (FIG. 1D) GFP-DYRK1B wild type and proline to alanine mutants were co-expressed with FLAG-PHD1 in U251 glioma cells. Cell lysates were analyzed for proline hydroxylation by immunoprecipitation using hydroxyl proline antibody followed by western blot for GFP-DYRK1B. Cell lysates were also analyzed for the interaction between GFPDYRK1B and FLAG-PHD1 by FLAG immunoprecipitation followed by western blot for GFP-DYRK1B. (FIG. 1E) Glioma cells were co-transfected with GFP-DYRK1B wild type or P332A, P333A, K140R (kinase dead) mutants, HA-VHL and FLAG-ID2. Cellular lysates were analyzed for ID2-T27 phosphorylation and for the interaction between ID2 and VHL by FLAG immunoprecipitation followed by western blot for HA-VHL. (FIG. 1F) FLAG-tagged recombinant ID2 was used in kinase assays in vitro including GFP-DYRK1B proteins immunoprecipitated from U87 cells expressing DYRK1B wild type or P332A, P333A and K140R mutants. After DYRK1 kinase assay, the FLAG-ID2 substrate was used in an in vitro binding assay with the VHL-ELOC-ELOB (VCB)-CUL2 complex. FLAG-ID2 immunoprecipitates were tested by western blot for the presence of bound VHL, phospho-ID2-T27 and FLAG-ID2. (FIG. 1G) GFP-DYRK1B wild type or P332A, P333A and K140R mutants were expressed in U87 cells. Cellular lysates were analyzed for total levels and phosphorylation of endogenous DYRK1 kinase substrates as indicated using total protein and phospho-site specific antibodies. WCL, whole cellular lysates. See also FIGS. 8, 9 and 15.

FIGS. 2A-F show that hydroxylation of P332 in DYRK1B and P380 in DYRK1A is necessary for kinase activity. (FIG. 2A) FLAG-PHD1 and GFP-DYRK1B wild type or P332A, P333A and K140R mutants were transfected into U87. Cellular lysates were analyzed by immunoprecipitation using anti-phospho-tyrosine or anti-hydroxyl proline antibody, respectively followed by western blot for GFP-DYRK1B. Cellular lysates were also analyzed for the interaction between DYRK1B and PHD1 by FLAG immunoprecipitation followed by western blot for DYRK1B. (FIG. 2B) FLAG-PHD1 and GFP-DYRK1A wild type or P380A and K188R mutants were expressed in U87 cells. Cellular lysates were analyzed by immunoprecipitation using phospho-tyrosine or hydroxyl proline antibody, respectively followed by western blot for DYRK1A. Cellular lysates were also analyzed for the interaction between DYRK1A and PHD1 by FLAG immunoprecipitation followed by western blot for DYRK1A. (FIG. 2C) Glioma cells were transfected with FLAG-DYRK1B wild type or FLAG-DYRK1B-P332A. Cells were treated with harmine (10 μM), CX-4945 (1 μM) for 8 hours or DMOG (1 mM) and CoCl₂ (150 μM) for 12 hours. Cellular lysates were analyzed by immunoprecipitation using hydroxyl proline or phospho-tyrosine antibody, respectively followed by western blot for DYRK1B. FLAG-DYRK1B proteins were also immunoprecipitated using FLAG antibody and used in a kinase assay towards recombinant ID2 in vitro. (FIG. 2D) GFP-DYRK1B wild type or P332A and P333A mutants were co-transfected with FLAG-PHD1 and treated with vehicle or DMOG. Cell lysates were analyzed by immunoprecipitation using hydroxyl proline or phospho-tyrosine antibody, respectively followed by western blot for DYRK1B. FAK is shown as a control. (FIG. 2E) FLAGDYRK1B wild type was subjected to in vitro transcription/translation for the indicated times. Reactions were immunoprecipitated with FLAG antibody followed by western blot using hydroxyl proline, phospho-tyrosine and FLAG antibodies. (FIG. 2F) Sequential double immuno-precipitation using hydroxyl-proline antibody followed by FLAG antibody from cells transfected with FLAG-DYRK1B wild type or FLAG-DYRK1B-P332A in the presence of HA-PHD1. Immunoprecipitates were analyzed by western blot using phosphotyrosine and FLAG antibody. WCL, whole cellular lysate. See also FIGS. 8, 9 and 15.

FIGS. 3A-D show that loss of hydroxylation of P332 alters the DYRK1B-chaperone complex and protein kinase maturation. (FIG. 3A) FLAG-DYRK1B wild type and mutants P332A, P333A or K140R were transfected into U87. FLAG-DYRK1B proteins were immuno-precipitated using FLAG antibody and analyzed for the interaction with endogenous CDC37 and HSP90. (FIG. 3B) FLAG-DYRK1B wild type and mutants P332A or K140R were transfected in glioma cells. Sub-cellular fractions were analyzed by western blot using markers as indicated. WCL, whole cellular lysate. (FIG. 3C) GFP-DYRK1B wild type and mutants P332A, P333A or K140R were transfected in glioma cells. GFP localization was examined by fluorescence microscopy. Nuclei were counterstained with DAPI. Scale bar: 50 μm. (FIG. 3D) Higher magnification of the areas outlined in (FIG. 3C) for GFP-DYRK1B-P332A (left panels) and GFP-DYRK1B-K140R (right panels). Scale bar: 25 μm.

FIGS. 4A-E shows that prolyl hydroxylation of DYRK1 kinases enhances VHL^{CRL} function. (FIG. 4A) DYRK1B wild type and DYRK1B-P332A proteins were immunoprecipitated from cells treated with vehicle or CoCl₂ and used in kinase assay towards recombinant FLAG-ID2 (in vitro kinase assay). The ID2 protein from the kinase reactions was used to challenge VBC-CUL2 complex integrity and interaction with HIF2α (obtained from cells transfected with HA-HIF2α in absence or presence of CoCl₂). GST pulldown was used to capture GST-VHL and CUL2 or FLAG-ID2 and the interaction between GST-VHL and HIF2α, in vitro binding/GST pull-down (see also FIG. 10D for HIF2α hydroxyl-proline immunoprecipitation). Phosphorylation of ID2-T27 was monitored by western blot. Proline hydroxylation of DYRK1B proteins was monitored by hydroxyl-proline immunoprecipitation followed by western blot for FLAG-DYRK1B. The total amount of proteins used in the reactions was also analyzed. (FIG. 4B) ID2 wild type, ID2-T27A expressing plasmids or the empty vector were transiently transfected in U87. Endogenous HIF2α, AURKA, Cyclin D1, and ZHX2 proteins were analyzed by western blot after 48 hrs. (FIG. 4C) U87 cells were infected with shRNA targeting ID2 or control lentivirus. 48 hrs later endogenous HIF2α, AURKA, Cyclin D1, and ZHX2 proteins were analyzed by western blot. (FIG. 4D) U87 cells were transfected with FLAG-DYRK1B wild type, DYRK1B-P332A or DYRK1B-K140R expressing plasmids or the empty vector. VHL was included as control for the effect of DYRK1B proteins on VHL substrates. Endogenous HIF2α, AURKA, Cyclin D1 and ZHX2 were analyzed by western blot. (FIG. 4E) GFP-DYRK1B wild type, GFP-DYRK1BP332A, GFP-DYRK1B-P333A or GFP-DYRK1B-K140R expressing plasmids were cotransfected with MYC-ubiquitin into U87 cells. HA-VHL was transfected as a positive control for the extent of ubiquitylation of the VHL substrates. Cells were treated with MG132 for 6 h and cellular lysates were immunoprecipitated with MYC antibody followed by western blot for the indicated proteins (WCL, whole cellular lysate is shown in FIG. 10F). See also FIG. 10.

FIG. 5A-D show that loss of prolyl hydroxylation impairs DYRK1B tumor suppression. (FIG. 5A) Western blot analysis of DYRK1B in U87 cells expressing doxycycline inducible DYRK1B wild type, DYRK1B-P332A or DYRK1B-P333A and treated with 1 μg ml⁻¹ doxycycline or vehicle for 36 hrs. (FIG. 5B) Tumor growth expressed as volume of individual sub-cutaneous xenografts of U87 cells expressing doxycycline inducible DYRK1B wild type, DYRK1BP333A or DYRK1B-P332A. Doxycycline treatment starts at day 0. Mice were euthanized after 25 days of treatment or when the tumor volume of control mice reached approximately 2,000 mm³ (n=4 mice per group; p-value: <0.0001 for DYRK1B wild type and DYRK1B-P333A versus DYRK1B-P332A treated with doxycycline, ANCOVA). (FIG. 5C) Tissue sections from experiment in (FIG. 5B) were immunostained using K167 antibody (red). Nuclei were counterstained with DAPI (blue). Scale bar, 100 μM. (FIG. 5D) Quantification of Ki67 positive cells from the experiment in (FIG. 5C); data in the histograms are means±s.d. (n=3 tumors; p-value 0.0008 and 3.31E-5 for DYRK1B wild type and DYRK1B-P333A versus DYRK1B-P332A treated with doxycycline, respectively; two-sided t-test, unequal variance). Asterisks indicate statistical significance. See also FIG. 11.

FIGS. 6A-F show structural representation of the elements in p38α and DYRK1B that regulate kinase activity. (FIG. 6A) Crystallographic structure of the kinase domain of p38α (PDB 5mtx) showing relative locations of active site, octyl-glucoside ligand binding site, and CMGC insert. Inset magnification shows the relative location of P242 that is subject to hydroxylation and the octyl-glucoside ligand. (FIG. 6B) Quantification of the change in the Van der Waals energy caused by 4-hydroxylation of P242 when octyl glucoside is bound (PDB 5mtx coordinates were used for van der Waals, electrostatic and internal energy calculations performed with ICM-Pro). (FIG. 6C) Electrostatic surface of crystallographic octylglucoside binding pocket in p38α. (FIG. 6D) Electrostatic surface of octylglucoside binding pocket in the model of DYRK1B (model built using ICM-Homology software and PDB 4mql as a template). (FIG. 6E) Immunoprecipitation using hydroxyl-proline antibody followed by FLAG antibody from cells transfected with FLAG-p38α wild type or FLAG-p38α-P242A in the presence of HA-PHD1. Immunoprecipitates were analyzed by western blot using FLAG and HA antibodies. WCL, whole cell lysate. (FIG. 6F) Cells transfected with FLAG-p38α wild type or FLAG-p38α-P242A were analyzed by western blot using p38α and phospho-T180/Y182-p38α antibodies. See also FIG. 12, FIG. 15, and FIG. 16.

FIGS. 7A-G show that prolyl hydroxylation by PHD enzymes is a key event in the maturation of kinases of the CMGC family. (FIG. 7A) FLAG-PHD1, FLAG-PHD2, FLAG-PHD3 or the empty vector were co-transfected with HA-DYRK3 in U87 glioma cells. Cellular lysates were analyzed for the interaction between PHD enzymes and DYRK3 by FLAG immunoprecipitation followed by western blot for HA-DYRK3 and tested for DYRK3 proline hydroxylation and tyrosine phosphorylation by hydroxyl-proline and phosphor-tyrosine immunoprecipitation, respectively followed by western blot for HA-DYRK3. (FIG. 7B) FLAG-PHD1, FLAG-PHD2, FLAG-PHD3 or the empty vector were co-transfected with HA-DYRK4 in U87 cells. Cellular lysates were analyzed for the interaction between PHD enzymes and DYRK4 by FLAG immunoprecipitation followed by western blot for HADYRK4 and tested for DYRK4 proline hydroxylation and tyrosine phosphorylation by hydroxyl-proline and phospho-tyrosine immunoprecipitation, respectively followed by western blot for HA-DYRK4. (FIG. 7C) FLAG-PHD1, FLAG-PHD2, FLAG-PHD3 or the empty vector were co-transfected with HA-GSK3β in U87 glioma cells. Cellular lysates were analyzed for the interaction between PHD enzymes and GSK3β by FLAG immunoprecipitation followed by western blot for HA-GSK3β and tested for GSK3β proline hydroxylation and tyrosine phosphorylation by hydroxyl-proline and phospho-tyrosine immunoprecipitation, respectively followed by western blot for HA-GSK3β. (FIG. 7D) FLAGPHD1 and HA-DYRK4 wild type or P323A were co-transfected into U87. Cellular lysates were analyzed for proline hydroxylation and tyrosine phosphorylation by immunoprecipitation using anti-HA antibody followed by western blot analysis using hydroxyl-proline or phospho-tyrosine antibody, respectively. CoCl₂ treatment was included as a control for PHD1 inhibition. (FIG. 7E) FLAG-PHD1 and HA-GSK3β wild type or P255A, P276A mutants or the double mutant HA-GSK3β-P255A-P276A were cotransfected into U87. Cell lysates were analyzed by immunoprecipitation using HA antibody followed by western blot using hydroxyl-proline or phospho-tyrosine antibody, respectively. CoCl₂ treatment was included as a control for PHD1 inhibition. In vitro kinase assay of immunoprecipitated HA-GSK3β proteins was performed using GST-MYC as substrate followed by western blot with phospho-MYC antibody. (FIG. 7F) HA-DYRK4 was subjected to in vitro transcription/translation for the indicated times. CoCl₂ was used to inhibit PHD enzymes. The reaction was immunoprecipitated with HA antibody followed by western blot using hydroxyl-proline and phospho-tyrosine antibody, respectively. (FIG. 7G) HA-GSK3β was subjected to in vitro transcription/translation for the indicated times. CoCl₂ was used to inhibit PHD enzymes. The reaction was immunoprecipitated with HA antibody followed by western blot using hydroxyl-proline and phospho-tyrosine antibody, respectively. WCL, whole cellular lysate. See also FIG. 13, FIG. 15, and FIG. 16.

FIGS. 8A-F (Related to FIG. 1) show DYRK1 kinase proline hydroxylation in vitro and mass spectrometry analysis of DYRK1B protein. (FIG. 8A) Silver staining of 100 ng of recombinant GSTDYRK1A and GST-DYRK1B purified from baculovirus, 100 ng of MYC-PHD1 purified from HEK-293T cells and 150 ng of ID2-FLAG purified from E. coli. Purified proteins were used for in vitro prolyl hydroxylation and kinase assays. (FIG. 8B) DYRK1A is prolyl hydroxylated in vitro by PHD1. Recombinant GST-DYRK1A produced in baculovirus was incubated in the presence or absence of PHD1 and immunoprecipitated using anti-hydroxyl proline antibody. Immunoprecipitated proteins were processed by SDS-PAGE and immunoblot using DYRK1A antibody. Total proteins in the in vitro reaction were included as control. (FIG. 8C) Bacterially expressed GST-DYRK1B was incubated in the presence or the absence of PHD1 and used to phosphorylate recombinant ID2-FLAG (in vitro kinase assay). Total proteins in the in vitro reaction were included as control. (FIG. 8D) Elution profiles of DYRK1B hydroxylated peptide IVEVLGIP#PAAMLDQAPK from cells expressing DYRK1B in the absence (upper panel) or presence (lower panel) of PHD1. The retention time profile of the peptide was generated by tracing a specific fragment, y11 (m/z 1154.5874) of peptide IVEVLGIP#PAAMLDQAPK (m/z 636.685) over time. The area underneath curve was automatically calculated by QualBrowser. Y axis was kept constant. (FIG. 8E) GFP-DYRK1B wild type and proline to alanine point mutants and double mutant P332A-P333A were co-expressed with PHD1 in U87 cells. Cell lysates were analyzed for the presence of proline hydroxylation and tyrosine phosphorylation by immunoprecipitation using anti-hydroxyl proline or anti-phospho-tyrosine antibody followed by western blot for GFP-DYRK1B. (FIG. 8F) Whole cellular lysate (WCL) from the experiment in E.

FIGS. 9A-B (Related to FIG. 2) show evolutionary conservation of proline 380 in DYRK1A and proline 332 in DYRK1B. (FIG. 9A) Amino acid sequence flanking P380 of DYRK1A is evolutionarily conserved. (FIG. 9B) Amino acid sequence flanking P332 of DYRK1B is evolutionarily conserved. The prolyl hydroxylation motif is boxed in red.

FIGS. 10A-F (Related to FIG. 4) show stoichiometry, functional analysis of hydroxylated P332 of DYRK1B and analysis of VHL substrates. (FIG. 10A) Hydroxyl-proline immunoprecipitation of HA-DYRK1B or HA-HIF2α expressed in 293T cells (500 μg of cellular lysates; 100% or 50% of DYRK1B or HIF2α reactions, respectively) and serial dilutions of total extract (μg: total μg of lysates) were processed by western blot using HA antibodies and analyzed by densitometry. Indicated is the fraction of hydroxylated proline in DYRK1B and HIF2α in the presence of FLAG-PHD1. (FIG. 10B) FLAG-DYRK1B proteins immunoprecipitated from U87 cells transfected with plasmids coding for DYRK1B wild type and the mutant P332G were used for hydroxyl-proline immunoprecipitation followed by western blot for FLAG. DYRK1B wild type and DYRK1BP332G were immunopurified from the same lysates and used in kinase assay in vitro towards recombinant ID2. (FIG. 10C) GFP-DYRK1A wild type or mutants P380A and K188R were transfected into U87. Cellular lysates were analyzed for total levels and phosphorylation of endogenous DYRK1 kinase substrates as described in FIG. 1G. (FIG. 10D) Western blot analysis of HIF2α from cells treated with vehicle or CoCl₂. HIF2α protein was used for GST-VHL pull-down experiment in FIG. 4A. (FIG. 10E) Interaction between AURKA, cyclin D1, ZHX2 with PHD3. U87 cells were transfected with plasmids expressing FLAG-PHD1, FLAG-PHD2, FLAG-PHD3 or the empty vector. Cellular lysates were immunoprecipitated using FLAG antibody followed by western blot for the indicated endogenous proteins (left panels). Reciprocal immunoprecipitations were also performed to test interaction of endogenous VHL substrates with FLAG-PHD proteins (right panels). (FIG. 10F) Western blot analysis of whole cellular lysates used in the ubiquitylation assay in FIG. 4E. WCL, whole cellular lysate.

FIG. 11 (Related to FIG. 5) shows histological analysis of DYRK1B expressing glioblastoma xenografts. Hematoxylin and Eosin staining shows enlarged tumor cells after doxycycline-induced expression of DYRK1B wild type and DYRK1B-P333A but not DYRK1BP332A. Scale bar: 100 μM.

FIG. 12A-B (Related to FIG. 6 and FIGS. 15 and 16) show that a prolyl hydroxylation motif is present in DYRK and MAPK kinase family members. (FIG. 12A) The prolyl hydroxylation motif (L/xGxP) is conserved across DYRK family members. (FIG. 12B) The prolyl hydroxylation motif (L/xGxP) is conserved across MAPK family members. Prolyl hydroxylation motif and auto-phosphorylation sites are boxed in red and blue, respectively.

FIGS. 13A-C(Related to FIG. 7 and FIG. 17) show that a prolyl hydroxylation motif is present in GSK, HIPK and CDK kinases. (FIG. 13A) The prolyl hydroxylation motif (L/xGxP) is conserved across GSK3 family members. (FIG. 13B) The prolyl hydroxylation motif (L/xGxP) is conserved across HIPK family members. (FIG. 13C) The prolyl hydroxylation motif (L/xGxP) is conserved across CDK family members. Prolyl hydroxylation motif and auto-phosphorylation sites are boxed in red and blue, respectively.

FIG. 14 (Related to FIG. 4) shows that PHD-dependent activation of DYRK1 kinases sustains tumor suppression by VCB^{CRL}. In cellular contexts that favor prolyl hydroxylase activity, PHD1 functions on newly synthesized DYRK1 kinases causing hydroxylation of proline 332 of DYRK1B (shown) and proline 380 of DYRK1A. This event is required for tyrosine phosphorylation and full activation of DYRK1. The activity of DYRK1 kinases on threonine 27 of ID2 imposes a functional constraint on ID2 ability to interact with the VHL-Elongin C-Elongin B (VCB complex) and inactivate the ubiquitin ligase activity of VCB^{CRL} by displacing CUL2 from the complex. With functional PHDs and DYRK1 kinases, the VCB^{CRL} ubiquitin ligase complex remains competent to efficiently ubiquitylate its substrates (HIF-α, Cyclin D1, ZHX2).

FIG. 15 (Related to FIGS. 1, 7 and FIGS. 9, 12) shows a list of prolyl hydroxylation motifs, sites of autophosphorylation in the activation loop and relative distance are reported for DYRK family members.

FIG. 16 (Related to FIG. 6 and FIG. 12) shows a list of prolyl hydroxylation motifs, sites of autophosphorylation in the activation loop and relative distance are reported for MAPK family members.

FIG. 17 (Related to FIG. 7 and FIG. 13) shows a list of prolyl hydroxylation motifs, sites of autophosphorylation in the activation loop and relative distance are reported for GSK3 and HIPK family members.

FIG. 18 (Related to FIG. 7 and FIG. 13) shows a list of prolyl hydroxylation motifs, sites of autophosphorylation in the activation loop and relative distance are reported for CDK family members.

FIG. 19 shows a schematic of hydroxylation of DYRK1.

FIG. 20 shows a list of reagents or resources utilized in the subject matter disclosed herein.

### DETAILED DESCRIPTION OF THE INVENTION

In certain aspects, the invention provides a method of modulating kinase activity, the method comprising modulating the prolyl hydroxylation status of said kinase.

In some embodiments, the kinase is a CMGC kinase. In some embodiments, the CMGC kinase is a dual specificity tyrosine-phosphorylation-regulated kinase (DYRK). In some embodiments, the DYRK kinase is DYRK1. In some embodiments, the DYRK1 kinase is DYRK1A. In some embodiments, the DYRK1 kinase is DYRK1B. In some embodiments, the DYRK kinase is DYRK2. In some embodiments, the DYRK kinase is DYRK3. In some embodiments, the DYRK kinase is DYRK4. In some embodiments, the CMGC kinase is a MAPK kinase. In some embodiments, the CMGC kinase is a GSK3 kinase. In some embodiments, the CMGC kinase is a HIPK kinase. In some embodiments, the CMGC kinase is a CDK kinase.

In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of the kinase. In some embodiments, DYRK1A is hydroxylated on proline 380 of the amino acid sequence. In some embodiments, DYRK1B is hydroxylated on proline 332 of the amino acid sequence.

In some embodiments, DYRK1B prolyl hydroxylation results in phosphorylation of ID2. In some embodiments, DYRK1B prolyl hydroxylation releases VHL ubiquitin ligase from ID2. In some embodiments, the modulation comprises increasing kinase activity. In some embodiments, the modulation comprises decreasing kinase activity.

In some embodiments, prolyl hydroxylation of the kinase increases kinase activity. In some embodiments, prolyl hydroxylation results in kinase autophosphorylation. In some embodiments, prolyl hydroxylation of the kinase results in phosphorylation of downstream targets of the kinase.

In some embodiments, prolyl hydroxylation of the kinase results in tumor growth suppression. In some embodiments, prolyl hydroxylation of the kinase results in glioma growth suppression.

In some embodiments, the modulator is a small molecule. In some embodiments, the modulator acts on a PHD hydroxylase to alter its activity toward the kinase. In some embodiments, the modulator is CoCl₂. In some embodiments, the modulator is dimethyloxalylglycine (DMOG). In some embodiments, the modulation comprises genetic engineering of the kinase or of a hydroxylase acting on the kinase.

In certain aspects, the invention provides a method of activating DYRK1A kinase, the method comprising promoting prolyl hydroxylation of the kinase. In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of DYRK1A. In some embodiments, DYRK1A is hydroxylated on proline residue 380 of the amino acid sequence. In some embodiments, prolyl hydroxylation of DYRK1A results in DYRK1A autophosphorylation.

In some embodiments, prolyl hydroxylation of DYRK1A results in phosphorylation of DYRK1A downstream targets. In some embodiments, prolyl hydroxylation of DYRK1A results in tumor growth suppression. In some embodiments, In some embodiments, prolyl hydroxylation of DYRK1A results in glioma growth suppression.

In some embodiments, the method comprises a small molecule activator. In some embodiments, the method comprises an activator acting on a PHD hydroxylase to alter its activity toward DYRK1A. In some embodiments, the method comprises genetic engineering of DYRK1A or genetic engineering of a hydroxylase acting on DYRK1A.

In certain aspects, the invention provides a method of activating DYRK1B kinase, the method comprising promoting prolyl hydroxylation of the kinase.

In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of DYRK1B. In some embodiments, DYRK1B is hydroxylated on proline residue 332 of the amino acid sequence. In some embodiments, prolyl hydroxylation of DYRK1B results in DYRK1B autophosphorylation. In some embodiments, DYRK1B prolyl hydroxylation results in phosphorylation of ID2. In some embodiments, prolyl hydroxylation of DYRK1B results in phosphorylation of downstream targets of the kinase. In some embodiments, DYRK1B prolyl hydroxylation releases VHL ubiquitin ligase from ID2.

In some embodiments, prolyl hydroxylation of DYRK1B results in tumor growth suppression. In some embodiments, prolyl hydroxylation of DYRK1B results in glioma growth suppression.

In some embodiments, the method comprises a small molecule activator. In some embodiments, the method comprises an activator acting on a PHD hydroxylase to alter its activity toward DYRK1B. In some embodiments, the method comprises genetic engineering of DYRK1B or genetic engineering of a hydroxylase acting on DYRK1B.

In certain aspects, the invention provides a method of treating cancer in a subject in need thereof, the method comprising administering to the subject a therapeutically effective amount of a composition capable of modulating kinase.

In some embodiments, the kinase is a CMGC kinase. In some embodiments, the CMGC kinase is a dual specificity tyrosine-phosphorylation-regulated kinase (DYRK). In some embodiments, the DYRK kinase is DYRK1. In some embodiments, the DYRK1 kinase is DYRK1A. In some embodiments, the DYRK1 kinase is DYRK1B. In some embodiments, the DYRK kinase is DYRK2. In some embodiments, the DYRK kinase is DYRK3. In some embodiments, the DYRK kinase is DYRK4. In some embodiments, the CMGC kinase is a MAPK kinase. In some embodiments, the CMGC kinase is a GSK3 kinase. In some embodiments, the CMGC kinase is a HIPK kinase. In some embodiments, the CMGC kinase is a CDK kinase.

In some embodiments, the composition promotes prolyl hydroxylation of said kinase. In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of the kinase. In some embodiments, DYRK1A is hydroxylated on proline 380 of the amino acid sequence. In some embodiments, DYRK1B is hydroxylated on proline 332 of the amino acid sequence. In some embodiments, DYRK1B prolyl hydroxylation results in phosphorylation of ID2. In some embodiments, DYRK1B prolyl hydroxylation releases VHL ubiquitin ligase from ID2.

In some embodiments, the modulation comprises increasing kinase activity. In some embodiments, the modulation comprises decreasing kinase activity. In some embodiments, prolyl hydroxylation of the kinase increases kinase activity. In some embodiments, prolyl hydroxylation of the kinase results in the kinase autophosphorylation.

In some embodiments, prolyl hydroxylation of the kinase results in phosphorylation of downstream targets of the kinase. In some embodiments, prolyl hydroxylation of the kinase results in tumor growth suppression. In some embodiments, the cancer is a glioma. In some embodiments, the cancer is a breast cancer. In some embodiments the cancer is a prostate cancer. In some embodiments, the cancer is a lung cancer. In some embodiments, the cancer is a bladder cancer. In some embodiments, the cancer is a colorectal cancer. In some embodiments, the cancer is the cancer is melanoma. In some embodiments, the cancer is a kidney cancer. In some embodiments, the modulator is a small molecule.

In some embodiments, the modulator acts on a PHD hydroxylase to alter its activity toward the kinase. In some embodiments, the modulator is CoCl₂. In some embodiments, the modulator is dimethyloxalylglycine (DMOG). In some embodiments, the modulation comprises genetic engineering of the kinase or genetic engineering of a hydroxylase acting on the kinase.

In certain aspects, the invention provides a method for decreasing tumor size in a subject in need thereof, the method comprising administering to the subject a therapeutically effective amount of a composition capable of modulating kinase activity.

In some embodiments, the kinase is a CMGC kinase. In some embodiments, the CMGC kinase is a dual specificity tyrosine-phosphorylation-regulated kinase (DYRK). In some embodiments, the DYRK kinase is DYRK1. In some embodiments, the DYRK1 kinase is DYRK1A. In some embodiments, the DYRK1 kinase is DYRK1B. In some embodiments, the DYRK kinase is DYRK2. In some embodiments, the DYRK kinase is DYRK3. In some embodiments, the DYRK kinase is DYRK4. In some embodiments, the CMGC kinase is a MAPK kinase. In some embodiments, the CMGC kinase is a GSK3 kinase. In some embodiments, the CMGC kinase is a HIPK kinase. In some embodiments, the CMGC kinase is a CDK kinase.

In some embodiments, the composition promotes prolyl hydroxylation of said kinase. In some embodiments, the prolyl hydroxylation is achieved by a PHD hydroxylase. In some embodiments, the PHD hydroxylase is PHD1. In some embodiments, the prolyl hydroxylation is in the L/xGxP consensus sequence of the kinase. In some embodiments, DYRK1A is hydroxylated on proline 380 of the amino acid sequence. In some embodiments, DYRK1B is hydroxylated on proline 332 of the amino acid sequence. In some embodiments, DYRK1B prolyl hydroxylation results in phosphorylation of ID2. In some embodiments, DYRK1B prolyl hydroxylation releases VHL ubiquitin ligase from ID2.

In some embodiments, the modulation comprises increasing kinase activity. In some embodiments, the modulation comprises decreasing kinase activity. In some embodiments, prolyl hydroxylation of the kinase increases kinase activity. In some embodiments, prolyl hydroxylation of the kinase results in the kinase autophosphorylation. In some embodiments, prolyl hydroxylation of the kinase results in phosphorylation of downstream targets of the kinase.

In some embodiments, prolyl hydroxylation of the kinase results in tumor growth suppression. In some embodiments, the tumor is a glioma tumor. In some embodiments, the tumor is a tumor in the breast tissue. In some embodiments, the tumor is a tumor in the prostate tissue. In some embodiments, the tumor is a tumor in the lung tissue. In some embodiments, the tumor is a tumor in the bladder tissue. In some embodiments, the tumor is a tumor in the colorectal tissue. In some embodiments, the tumor is melanoma tumor. In some embodiments, the tumor is a tumor in the kidney.

In some embodiments, the modulator is a small molecule. In some embodiments, the modulator acts on a PHD hydroxylase to alter the activity of the hydroxylase toward the kinase. In some embodiments, the modulator is CoCl₂. In some embodiments, the modulator is dimethyloxalylglycine (DMOG). In some embodiments, the modulation comprises genetic engineering of the kinase or genetic engineering of a hydroxylase acting on the kinase.

Activation of dual specificity tyrosine-phosphorylation-regulated kinases 1A and 1B (DYRK1A and DYRK1B) requires prolyl-hydroxylation by PHD1 prolyl hydroxylase. Prolyl-hydroxylation of DYRK1 initiates a cascade of events leading to the release of molecular constraints on VHL ubiquitin ligase tumor suppressor function. However, hydroxylated proline/s and role of prolyl-hydroxylation in DYRK1 tyrosine autophosphorylation are unknown. We found that a highly conserved proline in the CMGC insert of DYRK1 kinase domain is hydroxylated by PHD1 and this event precedes tyrosine autophosphorylation. Mutation of the hydroxylation acceptor proline precludes tyrosine autophosphorylation and folding of DYRK1, resulting in a kinase unable to preserve VHL function and lacking glioma suppression activity. The consensus proline sequence is shared by most CMGC kinases and prolyl-hydroxylation is essential for catalytic activation. Thus, formation of prolyl-hydroxylated intermediates is a novel mechanism of kinase maturation and likely a general mechanism of regulation of CMGC kinases in eukaryotes.

In some embodiments, the subject matter described herein relates to an effective drug inhibitor of oncoprotein kinases for lethal cancer. In some embodiments, the subject matter described herein relates to an effective protein kinase inhibitors of the allosteric type.

In some embodiments, the subject matter described herein relates to the study of eukaryotic protein kinases. In some embodiments, the subject matter described herein relates to novel mechanisms of kinase activation. In some embodiments, the subject matter described herein relates to therapeutics for oncology. In some embodiments, the subject matter described herein relates to the design of protein kinase allosteric inhibitors.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Abstract

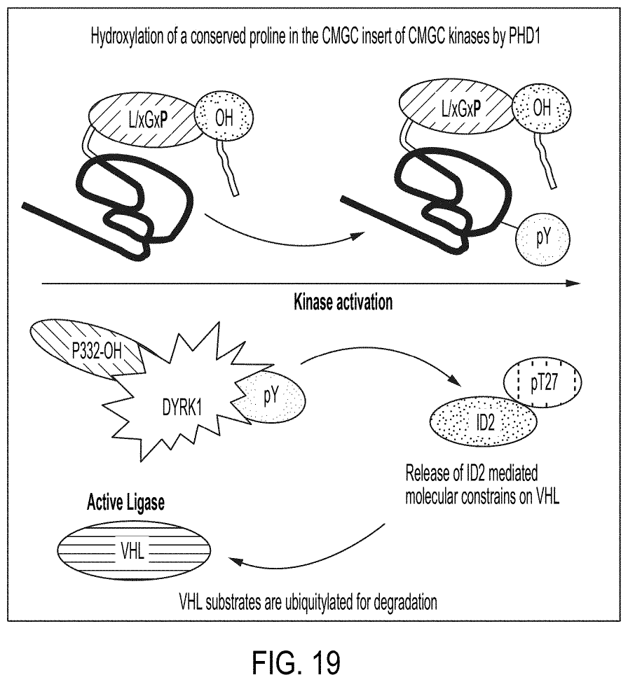

Activation of dual specificity tyrosine-phosphorylation-regulated kinases 1A and 1B (DYRK1A and DYRK1B) requires prolyl-hydroxylation by PHD1 prolyl hydroxylase. Prolyl-hydroxylation of DYRK1 initiates a cascade of events leading to the release of molecular constraints on VHL ubiquitin ligase tumor suppressor function. However, hydroxylated proline/s and role of prolyl-hydroxylation in DYRK1 tyrosine autophosphorylation are unknown. In one embodiment, the subject matter disclosed herein relates to a highly conserved proline in the CMGC insert of DYRK1 kinase domain, which is hydroxylated by PHD1 and this event precedes tyrosine autophosphorylation as shown in FIG. 19. Mutation of the hydroxylation acceptor proline precludes tyrosine autophosphorylation and folding of DYRK1, resulting in a kinase unable to preserve VHL function and lacking glioma suppression activity. The consensus proline sequence is shared by most CMGC kinases and prolyl-hydroxylation is essential for catalytic activation. Thus, formation of prolyl-hydroxylated intermediates is a novel mechanism of kinase maturation and likely a general mechanism of regulation of CMGC kinases in eukaryotes.

### Introduction

Proline hydroxylation is a common but still poorly understood protein modification (Gorres and Raines, 2010). The proline hydroxylation reaction is catalyzed by the 2-oxoglutarate and oxygen dependent dioxygenases PHD1, PHD2 and PHD3 (Semenza, 2001). PHD enzymes hydroxylate HIF1α and HIF2α proteins, thus promoting recognition and destruction of these transcription factors by the Von Hippel Lindau (VHL)-CUL-2 ubiquitin ligase and tumor suppressor (Kaelin and Ratcliffe, 2008). Recently, it was uncovered an additional layer of control of HIFα in cancer stem cells that is initiated by hydroxylation of the Dual-specificity tyrosine (Y) phosphorylation-regulated kinases 1A (DYRK1A) and 1B (DYRK1B) by PHD1 (Aranda et al., 2011; Lee et al., 2016). PHD1-mediated hydroxylation promoted DYRK1 kinase activity towards a conserved threonine (T27) of ID2, a protein that in its active unphosphorylated form drives the cancer stem cell state and multiple aspects of tumor aggressiveness (Lasorella et al., 2014). When phosphorylated by DYRK1 on T27, ID2 was unable to bind and disrupt the VHL-CUL2 ubiquitin ligase complex, thus restoring VHL function and HIFα protein degradation (Lee et al., 2016). However, this work did not identify the DYRK1 proline residue(s) that are directly modified by PHD1 hydroxylation. Consequently, the biochemical events linking PHD1-mediated prolyl hydroxylation of DYRK1 to maturation into a catalytically active kinase remain unknown.

DYRK1A and DYRK1B belong to an evolutionary conserved family of protein kinases, the CMGC group, which includes Cyclin dependent kinases (C), Mitogen activated protein kinases (M), Glycogen synthase kinases (G) and CDC-like kinases (C) (Manning et al., 2002). The DYRK kinase family is composed of five members including DYRK1A, DYRK1B, DYRK2, DYRK3 and DYRK4 (Aranda et al., 2011; Soundararajan et al., 2013). DYRK1A and DYRK1B are the most studied members of the DYRK family. DYRK1A is critically important in the development of the central nervous system (Dowjat et al., 2007; Mazur-Kolecka et al., 2012). DYRK1B has been implicated in promoting terminal differentiation in several systems including muscle cells and neural progenitors and is the causal mutation of a familial metabolic syndrome (Abu Jhaisha et al., 2017; Mercer et al., 2005). Multiple studies reported that both DYRK1A and DYRK1B inhibit proliferation and activate cellular quiescence programs (Aranda et al., 2011; Becker, 2012; Hammerle et al., 2011; Litovchick et al., 2011; Park et al., 2010; Yabut et al., 2010). These data are consistent with the tumor suppressor activity of DYRK1 in glioblastoma, which is primarily executed by suppressing ID2 and HIF2α-driven glioma stemness (Lee et al., 2016). It remains unclear whether the tumor suppressor activity of DYRK1 requires prolyl hydroxylation and involves the regulation of other oncoprotein substrates of VHL besides HIFα proteins.

Phosphorylation in the activation loop is a critical step for the conversion of inactive to active conformation common to most eukaryotic kinases (Nolen et al., 2004). Several mechanisms are involved in activation loop phosphorylation and in the most recent years autophosphorylation has been recognized as a highly prevalent mechanism of kinase self-activation (Beenstock et al., 2016). DYRK1A and DYRK1B are prototypic examples of protein kinases that require tyrosine autophosphorylation of the activation loop for catalytic activation (Himpel et al., 2000; Kentrup et al., 1996). Although autophosphorylation and transphosphorylation have been recognized as a hallmark of active conformations, the paradox of how inert non-phosphorylated molecules manage to catalyze the phospho-transfer reaction on their own activation loop sites has remained unsolved (Johnson et al., 1996). It was suggested that other mechanisms acting in trans might drive the transformation of nascent kinase polypeptides during or immediately after protein translation into a “prone-to-autophosphorylate conformation” that would create intermediate forms of the kinases as an essential step in the process of achieving full catalytic activity (Beenstock et al., 2016).

The finding that prolyl hydroxylation is sufficient to enhance the catalytic activity of DYRK1 kinases left unanswered the question of when prolyl hydroxylation and tyrosine autophosphorylation occur during DYRK1 maturation and whether they are causally connected. More importantly, findings have raised the question as to whether prolyl hydroxylation was a unique property of DYRK1A and DYRK1B or a more general mechanism for activation of protein kinases.

In one embodiment, the subject matter disclosed herein relates to the identification of a highly conserved proline in the kinase domain of DYRK1 that is hydroxylated by PHD1. Proline hydroxylation precedes and is indispensable for tyrosine autophosphorylation during translation of the DYRK1 polypeptide. The subject matter disclosed herein also relates to the finding that prolyl hydroxylation is necessary for DYRK1 tumor suppression. Finally, it is reported herein that prolyl hydroxylation by PHD1 occurs in a conserved domain of CMGC kinases and is an essential early event by which kinases of this large family acquire catalytic activity.

### Result

### Identification of the Proline Residue of DYRK1 Hydroxylated by PHD1

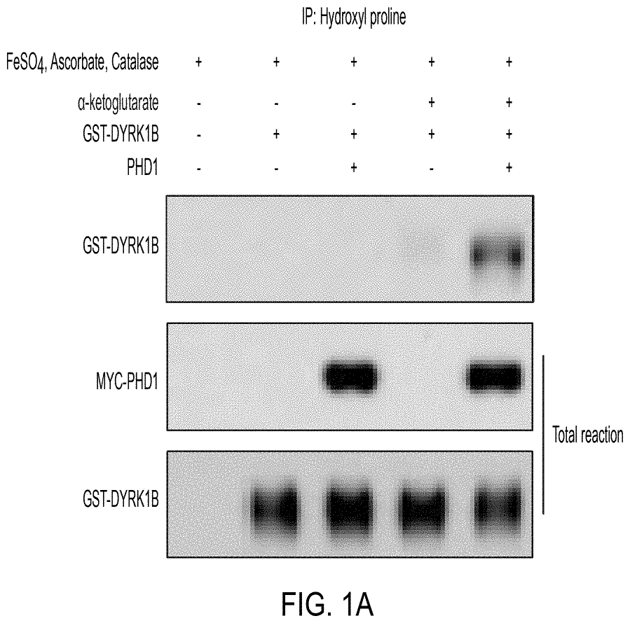

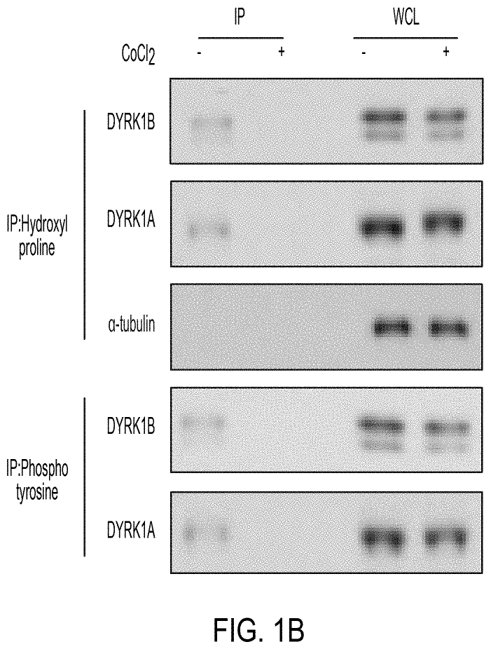

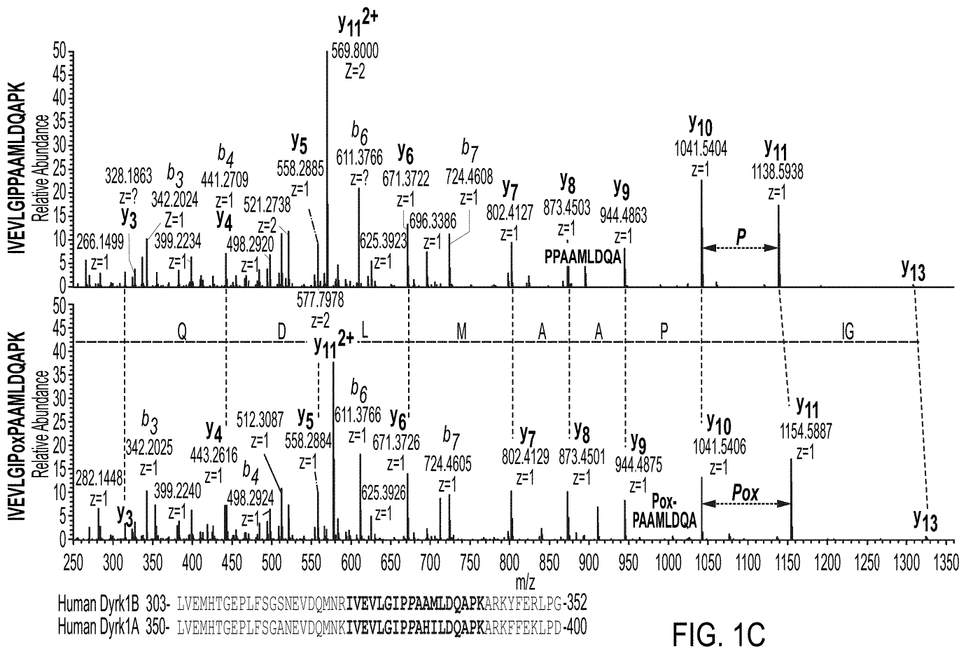

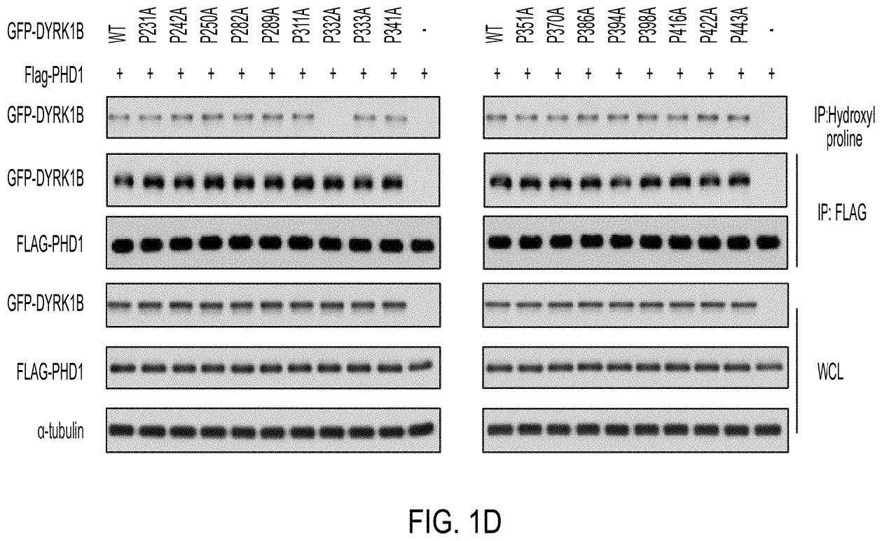

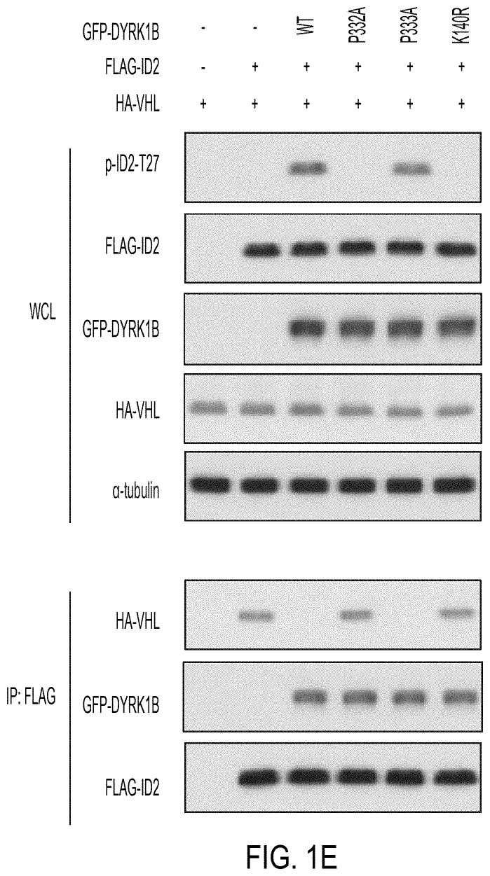

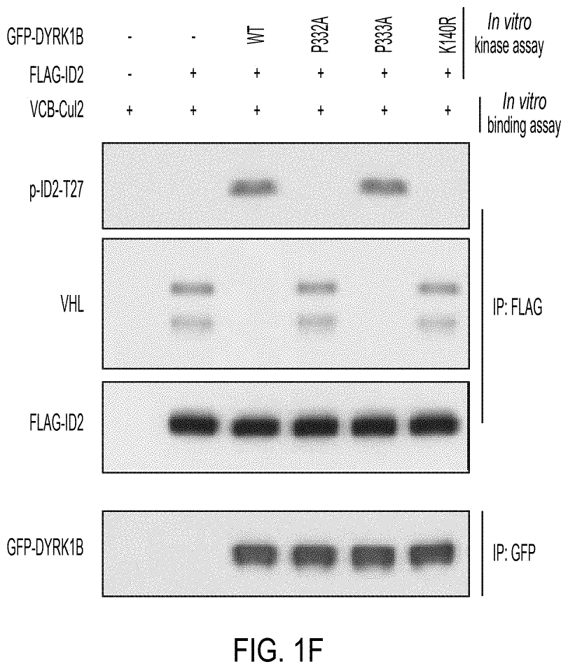

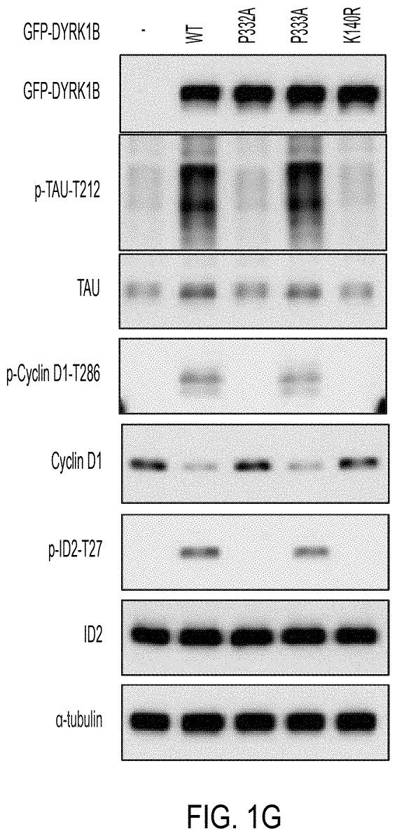

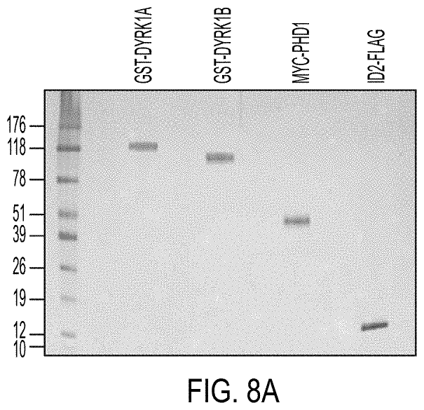

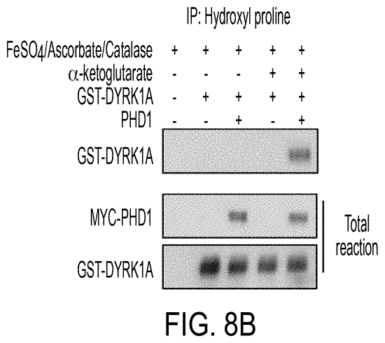

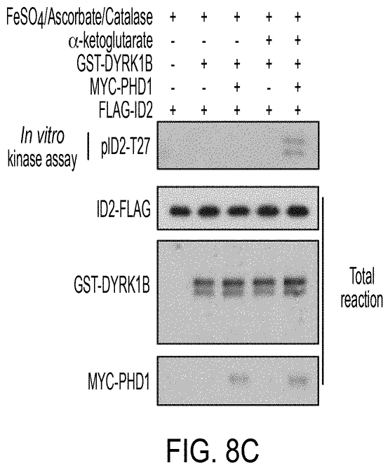

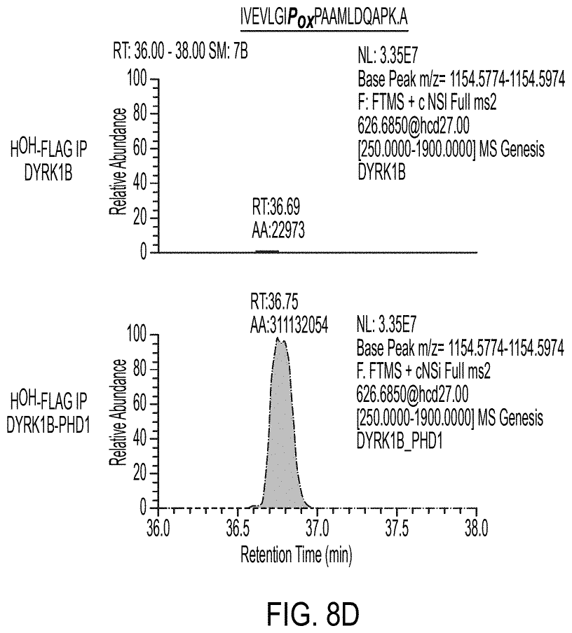

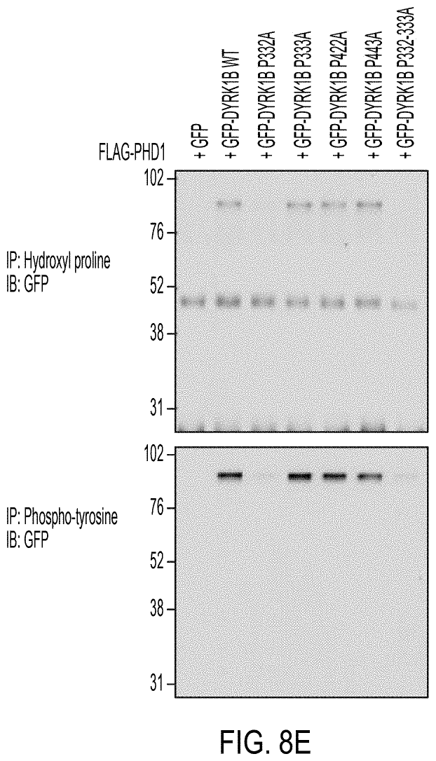

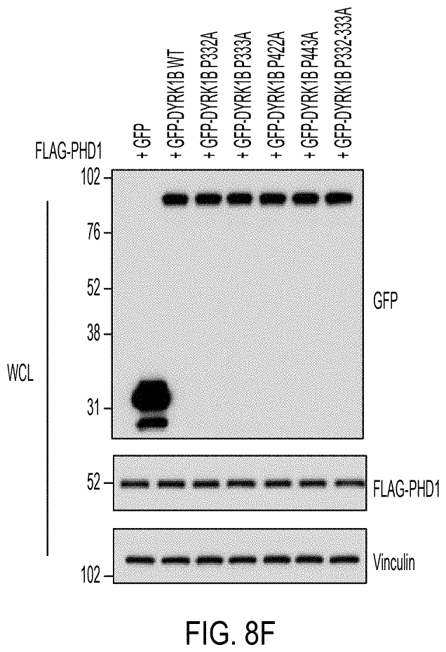

To determine whether DYRK1A and DYRK1B are direct substrates of prolyl hydroxylation by PHD1, an in vitro hydroxylation assay was developed to model PHD1-mediated proline hydroxylation of DYRK1. In this system, baculovirus-expressed GST-DYRK1A or GST-DYRK1B were incubated (FIG. 8A) in the presence or absence of recombinant MYC-tagged PHD1 in a reaction containing essential cofactors required for the enzymatic activity of PHD prolyl hydroxylases (Selak et al., 2005). As the PHD enzymes require α-ketoglutarate as co-substrate (MacKenzie et al., 2007; Schofield and Ratcliffe, 2004), the effect of α-ketoglutarate was also tested. Prolyl hydroxylated DYRK1 proteins were recovered by immunoprecipitation with hydroxyl-proline antibody followed by western blot for DYRK1A or DYRK1B. Efficient hydroxylation of GST-DYRK1A and GST-DYRK1B in vitro required both α-ketoglutarate and recombinant PHD1, thus indicating that prolyl hydroxylation of DYRK1 kinases is mediated by the α-ketoglutarate-dependent oxygenase activity of PHD1 (FIG. 1A, FIG. 8B). PHD1 and α-ketoglutarate were also required to induce the enzymatic activity of GST-DYRK1B purified from bacteria as shown by phosphorylation of recombinant FLAG-ID2 using in vitro kinase assay (FIG. 8C). Prolyl hydroxylated polypeptides of endogenous DYRK1A and DYRK1B are also present in vivo by performing immuno-precipitation using hydroxylproline antibody (FIG. 1B). Treatment of cells with the hypoxia-mimicking and PHD inhibitor CoCl₂ abolished prolyl hydroxylation and tyrosine autophosphorylation of DYRK1A and DYRK1B, thus underscoring the link between prolyl hydroxylation and tyrosine autophosphorylation, the latter being a marker of active DYRK1 kinases (FIG. 1B) (Himpel et al., 2000; Kentrup et al., 1996).

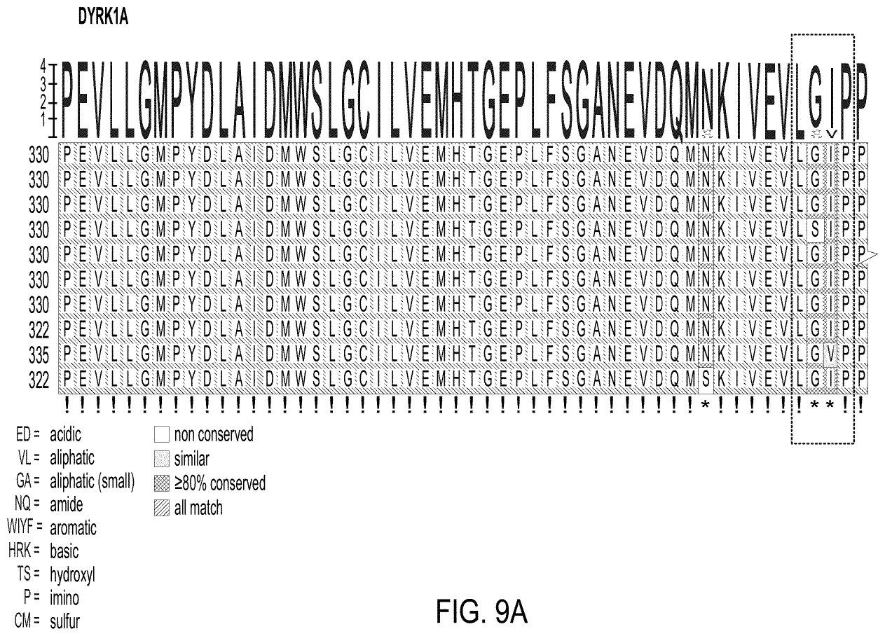

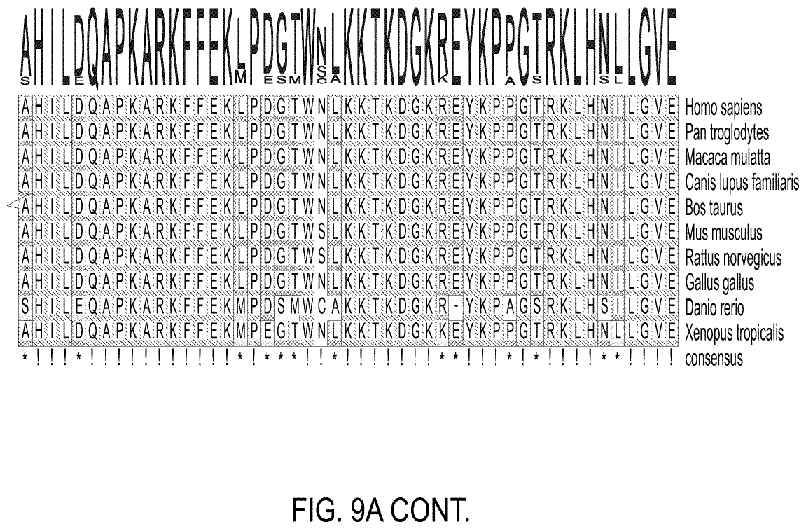

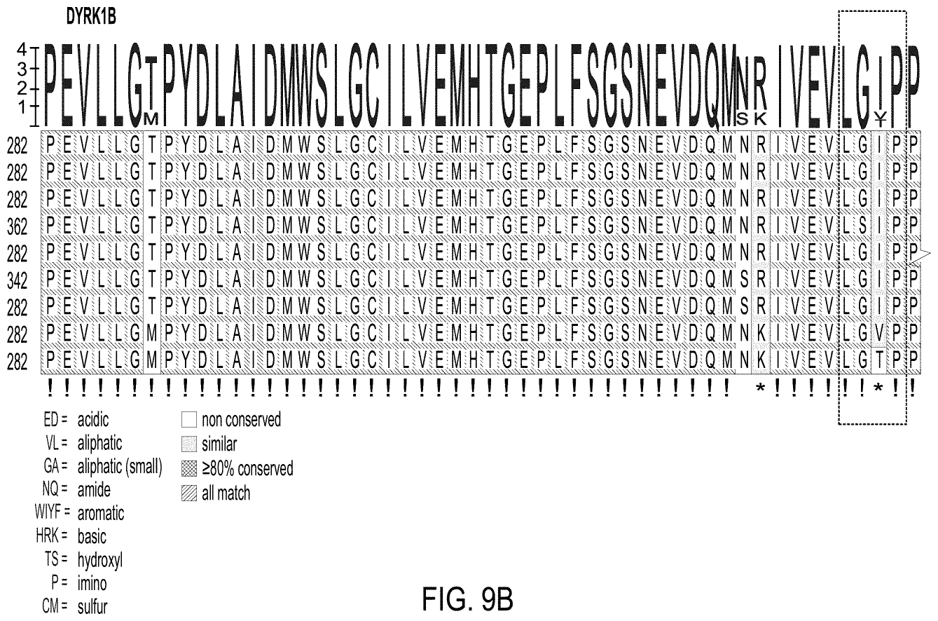

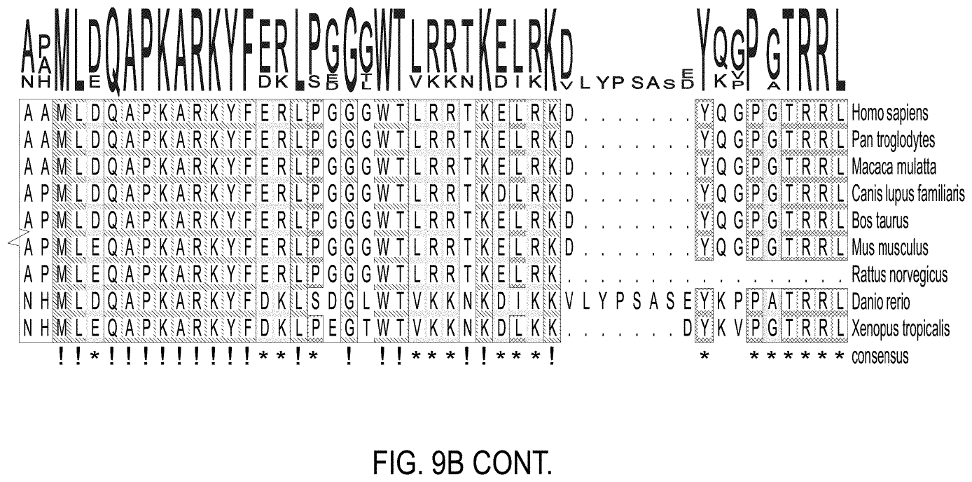

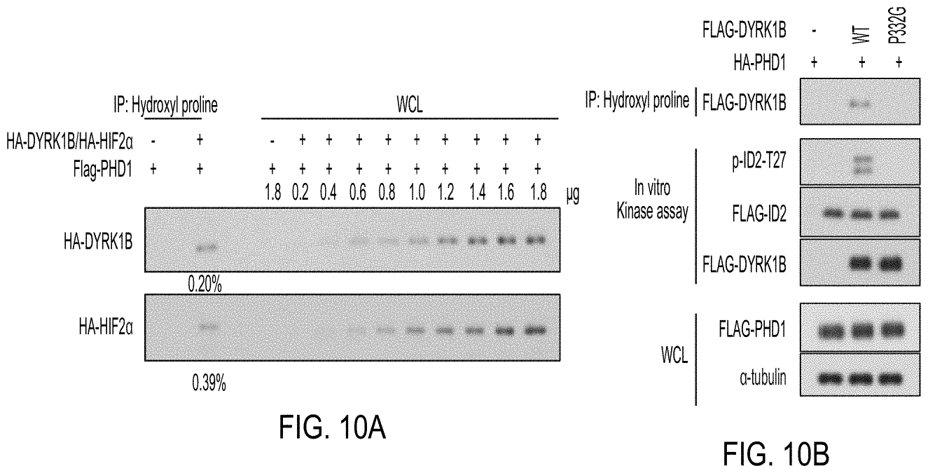

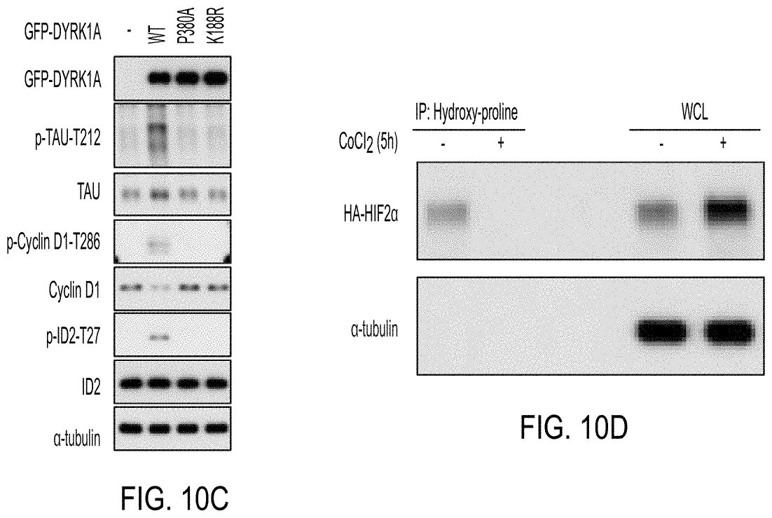

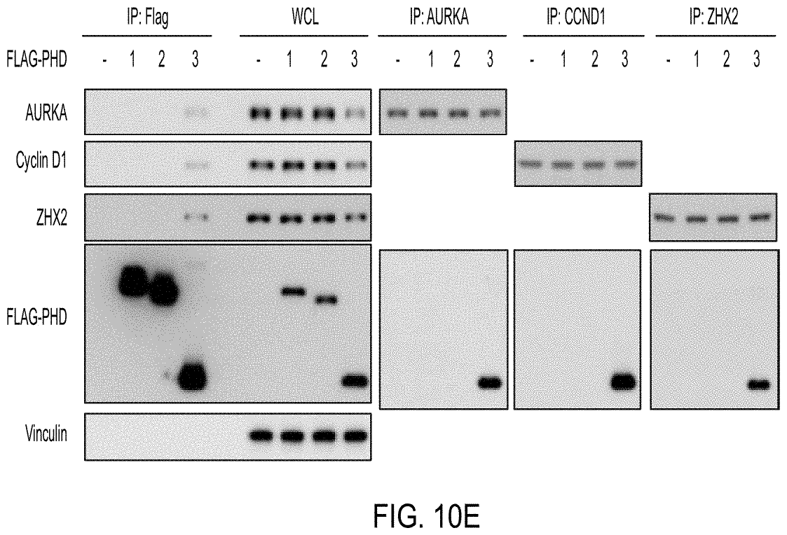

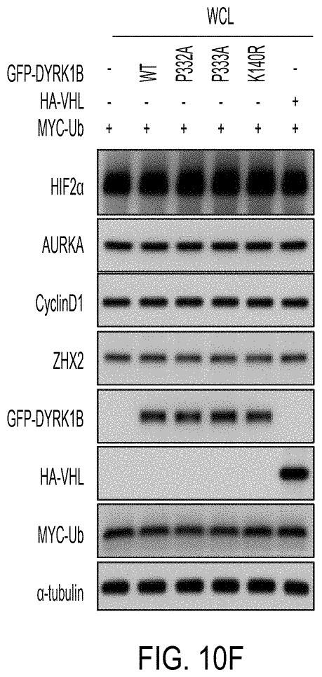

Next, two complementary and unbiased approaches were implemented to identify the proline residue/s on DYRK1 that are hydroxylated by PHD1. The two methods independently converged on the finding that PHD1 hydroxylates proline-332 (P332) in the kinase domain of DYRK1B. In the first analysis, glioma cells expressing FLAG-DYRK1B in the presence or absence of PHD1 were used. To enrich for proline-hydroxylated proteins, lysates were immunoprecipitated with an antibody against hydroxyl-proline followed by FLAG re-precipitation to specifically capture proline-hydroxylated FLAG-DYRK1B. As control for non-hydroxylated DYRK1B, FLAG-DYRK1B was immunoprecipitated with FLAG antibody from cells lacking PHD1. Immunoprecipitates were processed by LC-MS/MS to identify peptides containing hydroxylated proline/s. A peptide was identified with the following sequence: IVEVLGIP^{#}PAAMLDQAPK exhibiting +16-Da shift in the y-ion series at the y¹¹ ion, corresponding to fragments containing Pro-332 (FIG. 1C). From the LC-MS/MS, the retention time profiles of a specific fragment of the peptide IVEVLGIP^{ox}PAAMLDQAPK (y11, m/z 1154.5874) were traced in hydroxyl proline immunoprecipitations from cells transfected with FLAG-DYRK1B in the absence of PHD1 (FIG. 8D, upper panel) or in the presence of PHD1 (FIG. 1C, FIG. 8D, lower panel). The area under the curve was calculated and shows a peak of relative abundance corresponding to the peptide containing hydroxylated P332 in the sample from cells expressing DYRK1B and PHD1 but not in the samples expressing DYRK1B without PHD1 (FIG. 8D). The latter lacks detectable signal as proline hydroxylation was absent. In the second approach, 17 proline residues were individually mutated to alanine in or near the kinase domain of DYRK1B and performed hydroxyl-proline immunoprecipitation in the presence of PHD1 (FIG. 1D). It was found that wild-type DYRK1B and 16 proline-to-alanine mutants were equally immunoprecipitated by hydroxyl-proline antibody from cells expressing PHD1. Conversely, the P332A mutation abrogated PHD1-mediated proline hydroxylation of DYRK1B (FIG. 1D, IP: Hydroxyl-proline). The activity of PHD1 on P332 was highly specific, as mutation of the adjacent proline, P333A, did not affect DYRK1B hydroxylation, which was similarly abolished in the P332A mutant and double mutant P332A/P333A (FIG. 8E, F). However, whereas the DYRK1B P332A mutation abrogated DYRK1B prolyl hydroxylation, it did not affect the interaction between DYRK1B and PHD1 (FIG. 1D, IP: FLAG). P332 of DYRK1B is conserved in the kinase domain of the paralogous DYRK1A (P380) and both P332 in DYRK1B and P380 in DYRK1A are highly conserved throughout evolution (FIGS. 9A, B). To estimate the fraction of proline hydroxylated DYRK1B, HA-DYRK1B or HA-HIF2α, an established proline hydroxylated control, were immunoprecipitated using hydroxylproline antibody. Immunoprecipitates and increasing amounts of whole cellular lysates were processed by western blot using HA antibody. By densitometric comparison of bands from immunoprecipitation and cell lysates, it was established that 0.2% of HADYRK1B and 0.39% of HA-HIF2α are hydroxylated in the cell (FIG. 10A). These values are likely a considerable underestimation of the fraction of prolyl-hydroxylated DYRK1B and HIF2α in the cell as they reflect the limited immunoprecipitation efficiency of the anti-hydroxyl-proline antibody.

### DYRK1 Kinase Activity Requires Hydroxylation of P332 of DYRK1B and P380 of DYRK1A

Threonine 27 of ID2 is phosphorylated by DYRK1 kinases and the phospho-T27-ID2 protein cannot bind and disrupt the VHL^{CRL} ubiquitin ligase complex (Lee et al., 2016). Beside ID2, cyclin D1 and TAU are well-characterized substrates of DYRK1 kinases (Ashford et al., 2014; Yin et al., 2017). When phosphorylated on T286 by DYRK1, cyclin D1 is destabilized (Ashford et al., 2014). DYRK1 also phosphorylates T212 of the neuronal protein TAU and induces the abnormal protein aggregation typically associated with neurodegeneration (Yin et al., 2017). Here, it was asked whether prolyl hydroxylation of DYRK1 is required for kinase activity towards the ID2, cyclin D1 and TAU.

In glioma cells, expression of wild type DYRK1B or the DYRK1B-P333A mutant induced ID2-T27 phosphorylation and dissociation of ID2 from VHL (FIG. 1E). Conversely, prolyl hydroxylation deficient DYRK1B-P332A and kinase-inactive DYRK1B-K140R failed to phosphorylate T27 of ID2 and preserved the ID2-VHL interaction (FIG. 1E, IP FLAG). Loss of ID2-T27 phosphorylation by DYRK1B-P332A was not the consequence of an impaired interaction as DYRK1B wild type and DYRK1B-P332A exhibited very similar association with ID2 (FIG. 1E). Equally, in a combined DYRK1 kinase/ID2-VHL binding assay in vitro, DYRK1B-P332A was unable to phosphorylate ID2-T27 and preserved ID2 binding to VHL (Lee et al., 2016) (FIG. 1F). To further verify that loss of P332 hydroxylation impacts DYRK1B kinase activity, P332 was substituted with glycine (P332G). Mirroring DYRK1B-P332A mutation, DYRK1B-P332G lost proline hydroxylation and kinase activation as shown by hydroxyl-proline immunoprecipitation and absence of ID2 phosphorylation in vitro (FIG. 10B). The P332A mutation also abolished DYRK1B-mediated phosphorylation of Cyclin D1 on T286 and TAU on T212 (FIG. 1G). Consistent with the reported consequences of cyclin D1-T286 phosphorylation by DYRK1, wild type DYRK1B and DYRK1B-P333A reduced cyclin D1 protein levels but this effect was impaired by the expression of DYRK1B-P332A or DYRK1B-K140R (FIG. 1G). The P380A mutation of DYRK1A (corresponding to P332 in DYRK1B) exhibited similar effects (FIG. 10C). Taken together, these results show that prolyl hydroxylation of P332 of DYRK1B and P380 of DYRK1A are essential for DYRK1 kinase activity.

### Mechanism of Activation of DYRK1 Kinases by Prolyl Hydroxylation

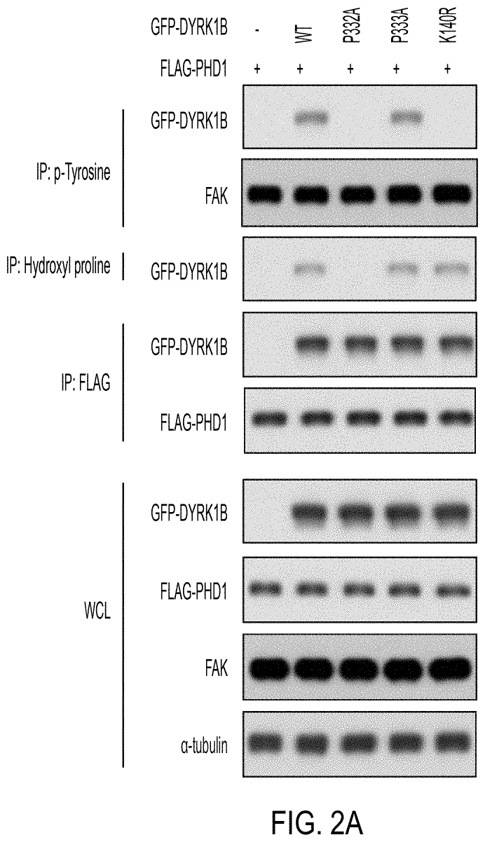

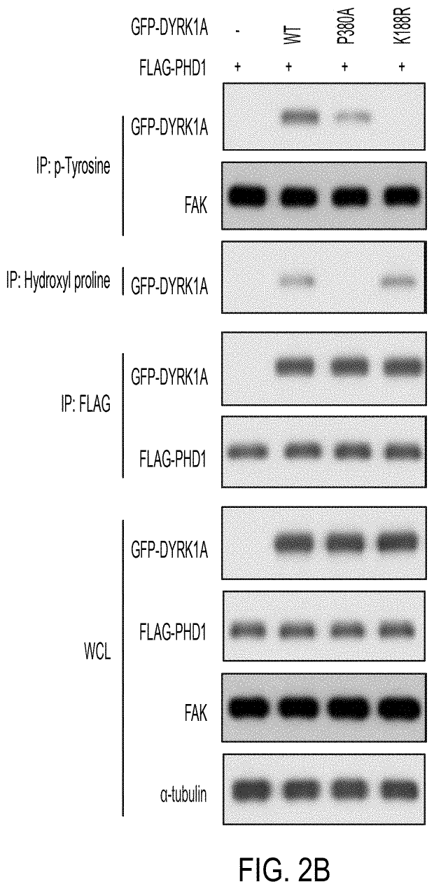

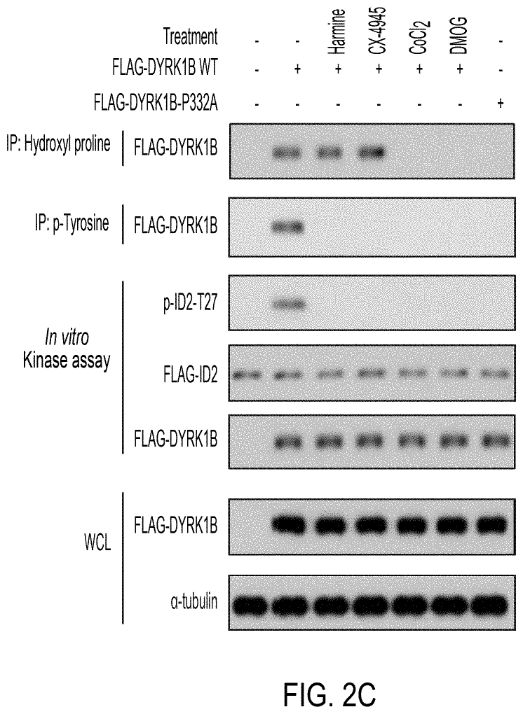

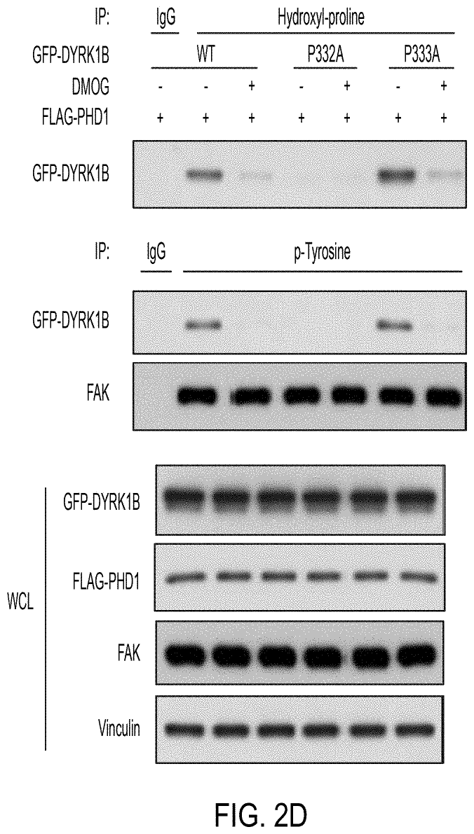

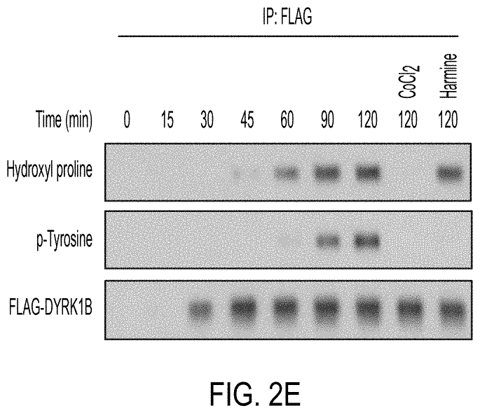

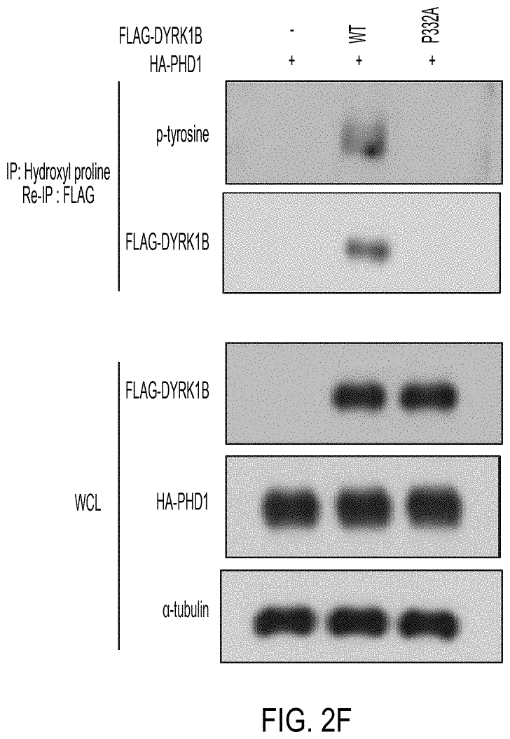

Autophosphorylation of a critical tyrosine in cis in the activation loop of DYRK1 kinases is an essential maturation event required for enzymatic activity. Tyrosine phosphorylation occurs during translation of DYRK1 before the kinase is released from the ribosome (Lochhead et al., 2005). The finding that prolyl hydroxylation is required for DYRK1 kinase activity left open the possibility that hydroxylation of P332 of DYRK1B (and P380 of DYRK1A) is also a translational event that either precedes or follows tyrosine autophosphorylation of DYRK1 kinases. It also remained to be established whether these two modifications, which are both essential for DYRK1 kinase activity, are causally linked or are independent and parallel events. To unravel the relationship between proline hydroxylation and tyrosine autophosphorylation of DYRK1 kinases, DYRK1B WT, the proline hydroxylation-deficient mutant P332A, control P333A and the kinase-dead K140R were first expressed in human glioma cells and performed immunoprecipitation using phospho-tyrosine antibody followed by western blot for DYRK1B. Lysates from the same cell cultures were analyzed for proline hydroxylation. Both DYRK1B wild type and the control P333A mutant were hydroxylated and tyrosine phosphorylated. However, loss of proline hydroxylation in DYRK1B-P332A abolished tyrosine autophosphorylation (FIG. 2A). This effect was comparable to the loss of tyrosine autophosphorylation in the ATP-binding site mutant DYRK1B-K140R (Jin et al., 2005). However, despite lacking tyrosine auto-phosphorylation, the inactive DYRK1B-K140R was still prolyl hydroxylated by PHD1 (FIG. 2A). Similarly, proline hydroxylation-deficient mutation of DYRK1A (DYRK1A-P380A) impaired tyrosine auto-phosphorylation whereas the ATP-binding site mutant, kinase-dead DYRK1A-K188R retained prolyl hydroxylation in the absence of tyrosine autophosphorylation (FIG. 2B). The interaction with PHD1 was maintained by all DYRK1 proteins regardless of prolyl hydroxylation and tyrosine autophosphorylation (FIGS. 2A, B). It was also asked herein whether inhibition of tyrosine auto-phosphorylation caused by harmine and CX-4945, two compounds that act as ATP competitive inhibitors of DYRK1 kinases (Ashford et al., 2016; Gockler et al., 2009; Kim et al., 2016), affected prolyl hydroxylation. Mimicking the properties of the kinase dead DYRK1 mutants, harmine and CX-4945 failed to perturb prolyl hydroxylation of DYRK1B, despite severely impairing tyrosine autophosphorylation and phosphorylation of the ID2-T27 substrate (FIG. 2C).

To investigate whether the enzymatic activity of PHD1 was required for tyrosine auto-phosphorylation of DYRK1, cells expressing DYRK1B wild type or DYRK1B-P332A and P333A were exposed to the prolyl hydroxylase inhibitors dimethyloxalylglycine (DMOG) or CoCl₂. Treatment of glioma cells expressing wild type DYRK1B with DMOG or CoCl₂ and cells expressing DYRK1B-P333A with DMOG caused loss of prolyl hydroxylation and tyrosine auto-phosphorylation (FIGS. 2C, D). Conversely, DYRK1B-P332A lost both prolyl hydroxylation and tyrosine autophosphorylation regardless of PHD1 activity (FIGS. 2C, D). Therefore, while PHD1-mediated proline hydroxylation of DYRK1 kinases was required for tyrosine auto-phosphorylation in the activation loop, loss of tyrosine autophosphorylation caused by mutation or pharmacological inhibition did not impair PHD1-mediated hydroxylation of the conserved prolines of DYRK1 kinases.

Having established a hierarchical mechanistic link between proline hydroxylation and tyrosine phosphorylation of DYRK1, the temporal relationship between the two events was investigated. A time-course assay was established for DYRK1B translation using the in vitro rabbit reticulocyte lysate transcription/translation system, in which it was possible to concurrently measure the level of proline hydroxylation and tyrosine auto-phosphorylation in the newly synthesized DYRK1B polypeptides. The analysis showed that proline hydroxylation was induced 45-60 min after the beginning of DYRK1B transcription/translation whereas tyrosine auto-phosphorylation became clearly detectable after 90 minutes (FIG. 2E). To verify that proline hydroxylated DYRK1B was concurrently phosphorylated on tyrosine, a sequential double immunoprecipitation was performed from cells co-expressing FLAG-DYRK1B or FLAG-DYRK1B-P332A in the presence of HA-PHD1. Cellular lysates were first immunoprecipitated using hydroxyl-proline antibody and collected on protein A/G agarose matrix. Hydroxyl-proline-containing proteins were then released from the matrix and proline-hydroxylated FLAG-DYRK1B was re-precipitated by FLAG antibodies. Western blot analysis using FLAG and phospho-tyrosine antibodies showed that proline hydroxylated molecules of DYRK1B wild type were also tyrosine phosphorylated. The hydroxyl-proline/FLAG sequential immunoprecipitation of DYRK1B-P332A mutant protein resulted in negative signals from either FLAG or phosphotyrosine western blot (FIG. 2F). Together, these results indicate that proline hydroxylation of DYRK1 is essential for the ensuing tyrosine auto-phosphorylation and full catalytic activation of DYRK1 kinases.

### Chaperone-Mediated Maturation of DYRK1B is Regulated by Prolyl Hydroxylation

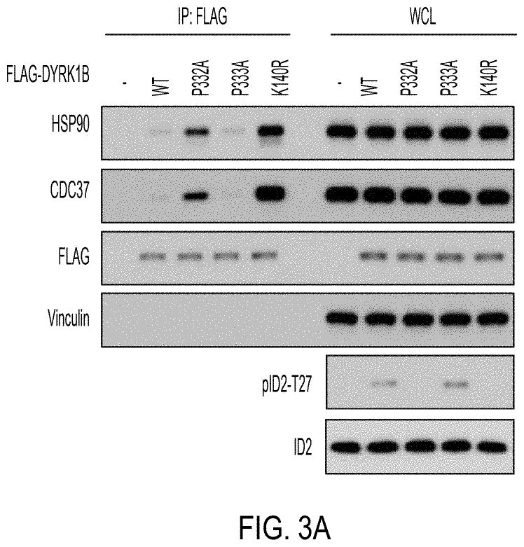

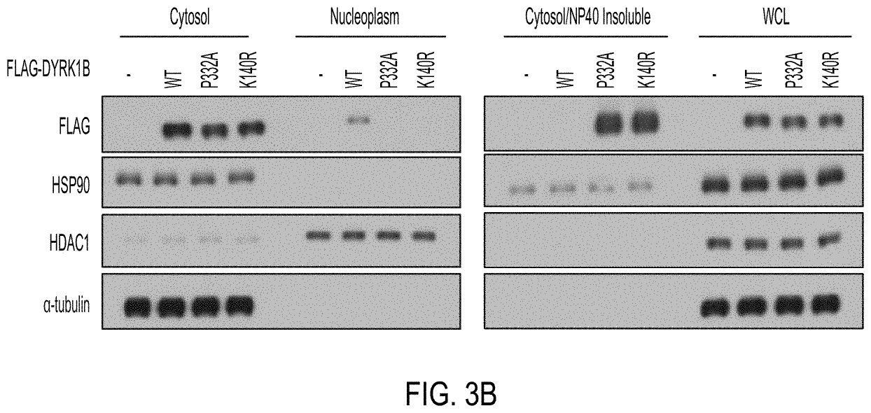

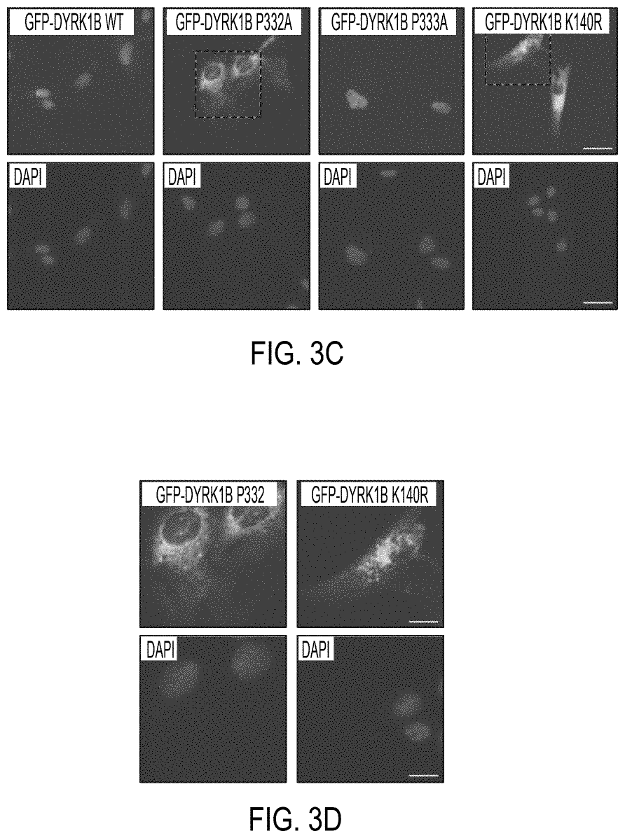

During protein translation, the CDC37-HSP90 chaperone complex transiently interacts with DYRK kinases to assist folding of the newly synthesized protein and maturation into a fully active kinase (Abu Jhaisha et al., 2017; Sonamoto et al., 2015; Taipale et al., 2012). Conversely, inactive DYRK kinases such as the kinase dead DYRK1 mutants that lack tyrosine auto-phosphorylation cannot mature into the active kinase conformation and are locked in stable complexes with CDC37-HSP90 (Abu Jhaisha et al., 2017; Sonamoto et al., 2015). As proline hydroxylation of DYRK1 precedes tyrosine auto-phosphorylation and kinase activation, it was asked whether preventing proline hydroxylation stabilizes the interaction with the CDC37-HSP90 chaperone. As expected, DYRK1B wild type and DYRK1B-P333A exhibited minimal interaction with CDC37-HSP90, reflecting the rapid turnover of these interactions. However, DYRK1B-P332A and DYRK1B-K140R mutant proteins exhibited a strong interaction with CDC37-HSP90 (FIG. 3A).

DYRK1 mutant proteins that cannot mature to active conformations aggregate in insoluble fractions of detergent-extracted cell homogenates visible under fluorescence microscopy as cytoplasmic aggregates (Abu Jhaisha et al., 2017). Consistently, DYRK1B-P332A mutant protein displayed a pattern similar to the kinase dead DYRK1B-K140R characterized by loss of nuclear localization and accumulation in the detergent-insoluble cellular fractions (FIG. 3B). Using fluorescence microscopy, it was verified that DYRK1B-P332A and DYRK1B-K140R proteins lost nuclear localization and remained cytoplasmic (FIGS. 3C, D). Conversely, DYRK1B wild type was recovered from the nuclear compartment and was absent in the detergent-insoluble fraction, a finding confirmed by fluorescence microscopy analysis of DYRK1B wild type and DYRK1B-P333A expressing cells (FIGS. 3B, C).

### Prolyl Hydroxylation of DYRK1 Protein Kinases Enhances VHL^{CRL} Function

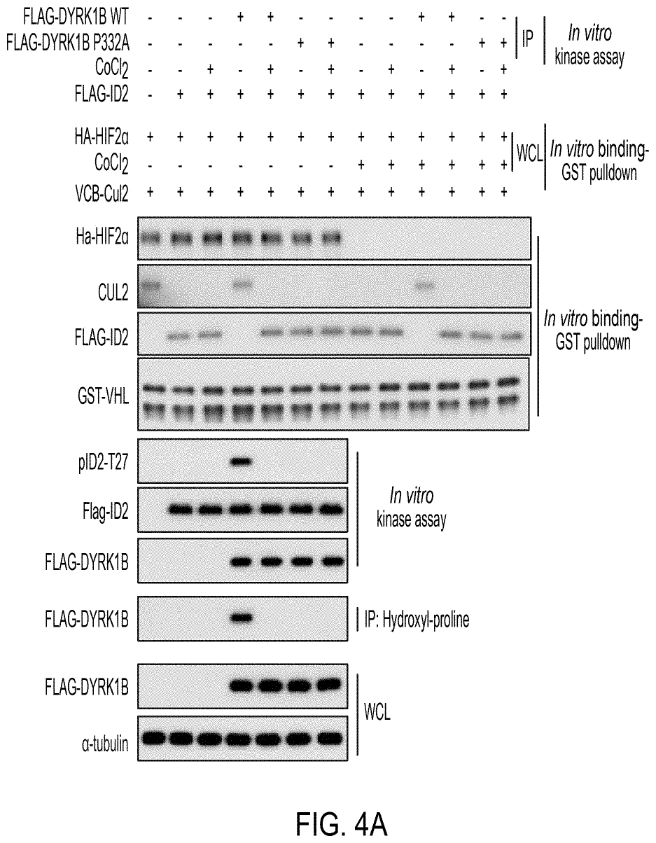

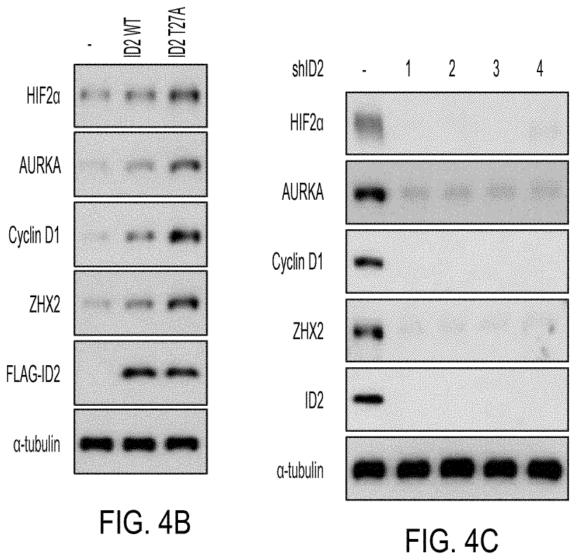

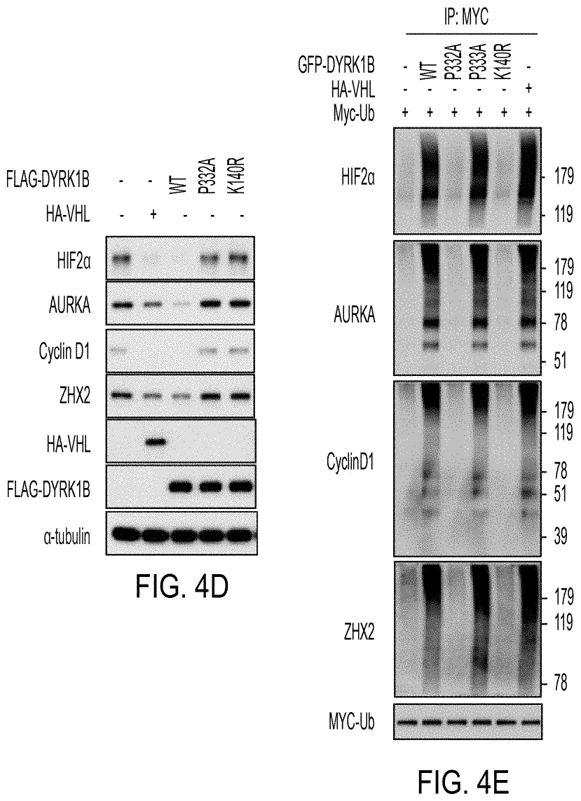

By phosphorylating ID2 on T27, DYRK1 kinases abolish ID2 ability to bind the VHL-Elongin C-Elongin B (VCB) complex and disrupt the interaction between VCB and the Cullin-2 (CUL2) adaptor, which is essential for ubiquitin ligase activity (Kershaw and Babon, 2015; Nguyen et al., 2015). A key oxygen-dependent function of DYRK1 is therefore to restrain ID2 and preserve the tumor suppressor activity of VHL towards HIFα and other oncoprotein substrates (Lee et al., 2016). Here, the consequences of DYRK1B-P332 hydroxylation were investigated on the integrity of the VCB-CUL2 complex. FLAG-tagged DYRK1B wild-type and P332A were immunoprecipitated from cells left untreated or treated with CoCl₂ to inhibit PHDs and DYRK1B hydroxylation. DYRK1B immunoprecipitates were used in a kinase reaction to phosphorylate T27 of recombinant FLAG-ID2 (FIG. 4A, middle panels, “In vitro kinase assay”). The FLAG-ID2 product of DYRK1B kinase reaction was then challenged for its ability to dissociate a pre-formed baculovirus-expressed VCB-CUL2 complex in an in vitro binding assay in which GST pulldown captured GST-VHL and components of the complex and HIF2α (FIG. 4A, upper panel, “In vitro binding, GST pulldown”). Parallel reactions were also analyzed to monitor prolyl hydroxylation of DYRK1B by hydroxyl-proline immunoprecipitation (FIG. 4A, lower panels, “IP: Hydroxyl-proline”). As expected, it was found that prolyl hydroxylated wild type DYRK1B efficiently phosphorylated ID2-T27 and prevented the interaction between ID2 and VCB, thus preserving the integrity of the VCB-CUL2 complex. Conversely, when DYRK1B-P332A immunoprecipitates were used to phosphorylate ID2 in vitro, the ID2 protein remained unphosphorylated and was able to bind VHL and dissociate CUL2 from VCB. ID2-mediated dissociation of CUL2 from VCB was also elicited if DYRK1B wild type or P332A immunoprecipitates included in the ID2 kinase reaction had been obtained from cells treated with CoCl₂ to inhibit DYRK1B prolyl hydroxylation (FIG. 4A). Regardless of the phosphorylation status of ID2 and the integrity of the VCB-CUL2 complex, GST-VHL was still able to interact in vitro with HIF2α obtained from untreated cells but VHL-HIF2α interaction was disrupted by treatment with CoCl₂ as the hypoxia mimicking agent prevented prolyl-hydroxylation of HIF2α (FIG. 4A, upper panels, FIG. 10D). Thus, prolyl hydroxylation of P332 of DYRK1B is required to phosphorylate ID2 and prevent ID2-mediated disruption of VCB-CUL2 complex and this effect is independent of the ability of VHL to recognize HIFα.

Beside acting on the HIFα proteins, VCB^{CRL} carries out its tumor suppressor function by ubiquitylation and destruction of other important oncoprotein substrates (Zhang and Zhang, 2018; Zhang and Yang, 2012), such as AURKA (Hasanov et al., 2017), cyclin D1 (Ashford et al., 2014; Bindra et al., 2002) and ZHX2 (Zhang et al., 2018). Resembling HIF2α, AURKA, cyclin D1 and ZHX2 undergo prolyl hydroxylation by PHDs, in particular PHD3 (FIG. 10E). In previous work, the effects of DYRK1 and ID2 were determined for the VHL-mediated ubiquitylation and destruction of HIFα proteins but whether the DYRK1-ID2 pathway impacts also other VHL substrates remained unknown. As first step it was asked whether AURKA, cyclin D1 and ZHX2 accumulated in the presence of ID2-T27A, a mutant protein that cannot be phosphorylated by DYRK1 (Lee et al., 2016). As for HIF2α, endogenous AURKA, cyclin D1 and ZHX2 proteins were elevated by the expression of ID2-T27A, but only minimally by wild-type ID2 (FIG. 4B). Conversely, HIF2α, AURKA, cyclin D1 and ZHX2 were all down-regulated in cells in which ID2 had been silenced by four independent shRNAs (FIG. 4C). Next, it was asked whether hydroxylation of P332 of DYRK1B was required for VHL-mediated ubiquitylation and destruction of these substrates. Expression of DYRK1B wild type, but not the P332A or the kinase-dead K140R mutants, reduced the steady state levels of HIF2α, AURKA, cyclin D1 and ZHX2 to an extent that was comparable to the levels observed after overexpression of VHL (FIG. 4D). Consistent with these observations, DYRK1B wild type and DYRK1B-P333A induced polyubiquitylation of HIF2α, AURKA, cyclin D1 and ZHX2 but DYRK1B-P332A and K140R were ineffective (FIG. 4E, FIG. 10F for whole cell lysate). Together, these results indicate that prolyl hydroxylation of the DYRK1 kinase operates upstream of VHL to regulate the ubiquitylation and accumulation of VHL substrates and is therefore part of the signaling pathway that operates through ID2 to control VHL ubiquitin ligase activity.

### Glioma Suppression by DYRK1B Requires P332 Hydroxylation

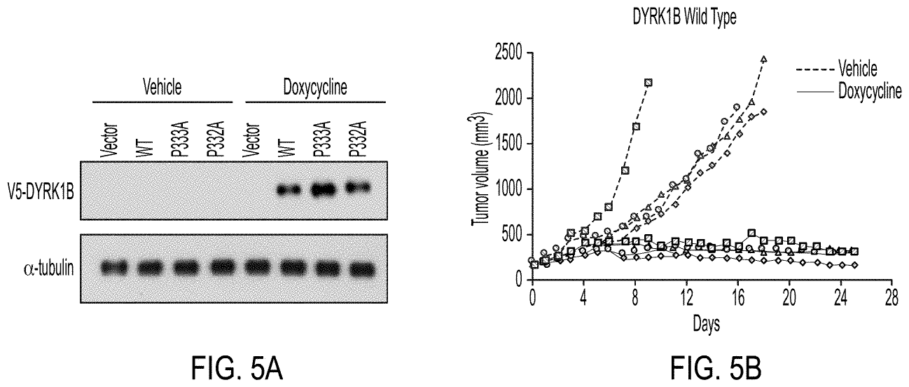

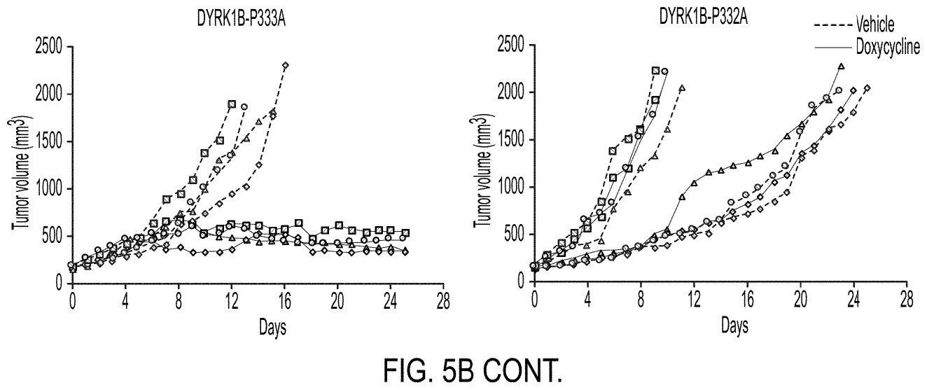

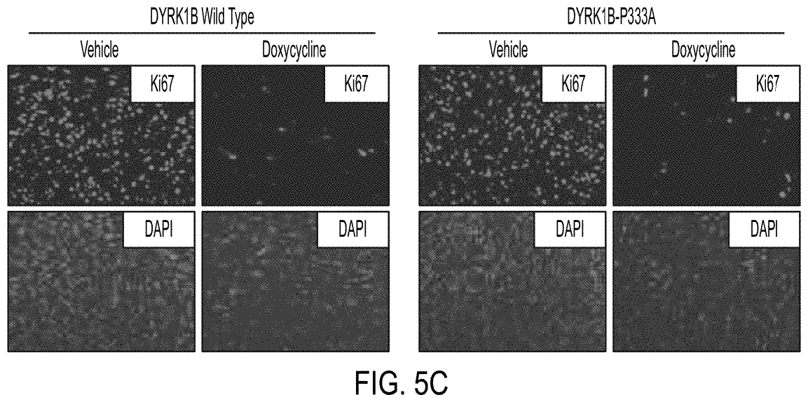

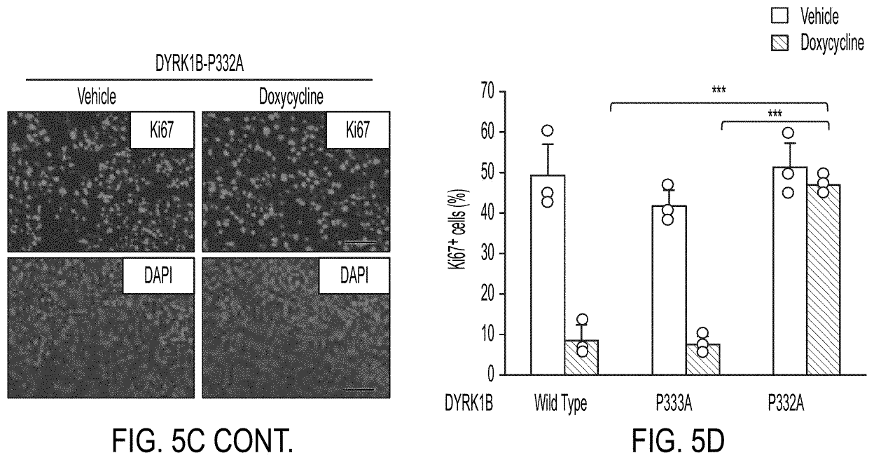

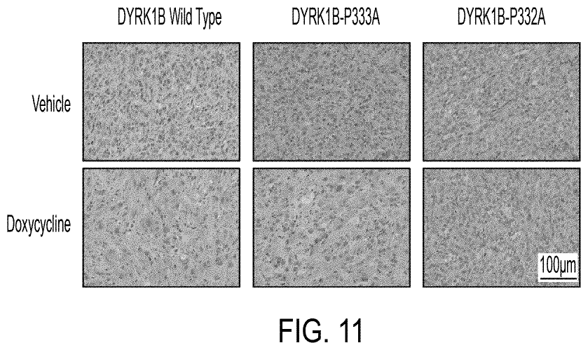

To determine the significance of proline hydroxylation for glioma progression, the effects of expressing DYRK1B wild type, DYRK1B-P332A or DYRK1B-P333A after tumors had been established were evaluated. To this aim, a doxycycline inducible system was used (Lee et al., 2016). In this model, tumors were established in the absence of doxycycline and the effects of the exogenous protein on tumor growth were determined after doxycycline induction (FIG. 5A). Animals bearing glioma undergoing treatment with vehicle exhibited a steady increase in tumor volume (FIG. 5B). However, treatment with doxycycline blocked growth of tumors expressing DYRK1B wild type and DYRK1B-P333A but not DYRK1B-P332A. Mice were euthanized after 25 days of doxycycline treatment, a time when all mice treated with vehicle and mice bearing tumors treated with doxycycline to express DYRK1B-P332A had been euthanized because tumor mass had reached the maximum volume allowed (p-value<0.0001, for DYRK1B wild type and DYRK1B-P333A versus DYRK1B-P332A treated with doxycycline). In the absence of doxycycline, tumors showed proliferation rates significantly higher than tumors induced to express DYRK1B wild type and DYRK1B-P333A as evaluated by Ki67 immunofluorescence. However, doxycycline treatment had no effect on DYRK1B-P332A expressing tumors (FIGS. 5C, D; p-value 0.0008 and 3.31E-5 for DYRK1B wild type and DYRK1B-P333A versus DYRK1B-P332A, respectively). Consistent with the proliferation results, Hematoxylin & Eosin staining showed that DYRK1B wild type and DYRK1B-P333A expressing cells acquired an enlarged, quiescent phenotype but cells expressing DYRK1B-P332A were morphologically indistinguishable from cells in untreated tumors (FIG. 11). Together, these results show that loss of hydroxylation of P332 abolishes the glioma suppression function of DYRK1B.

### Prolyl Hydroxylation is an Allosteric Modification Required for Autophosphorylation and Catalytic Activity of CMGC Protein Kinases

DYRK1A and DYRK1B are members of the DYRK family of kinases including also DYRK2, DYRK3 and DYRK4. They are part of the CMGC group of protein kinases. The evolutionarily conserved CMGC kinases include 62 members that perform critical cellular functions (Manning et al., 2002). Members of the CMGC kinases depend on autophosphorylation on the activation loop, generally on tyrosine or threonine, for their activation and a “prone-to autophosphorylation conformation” has been proposed to be part of the activation process (Beenstock et al., 2016). Therefore, without being bound by theory, hydroxylation of a critical proline might be a general event leading to autophosphorylation in the activation loop of CMGC kinases.

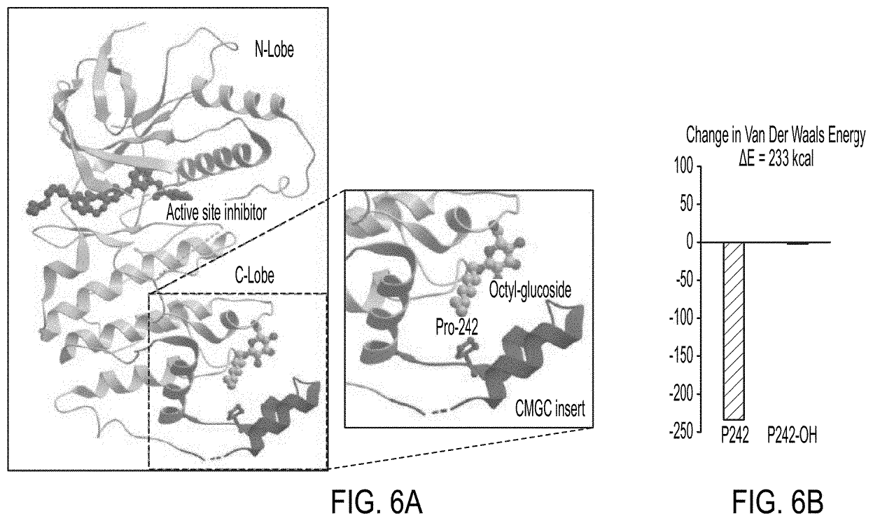

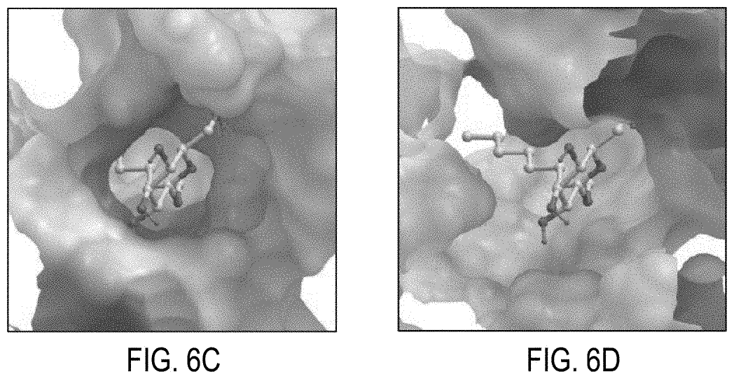

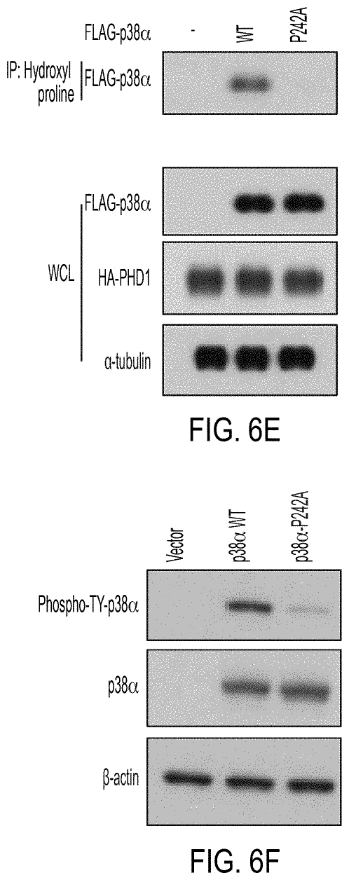

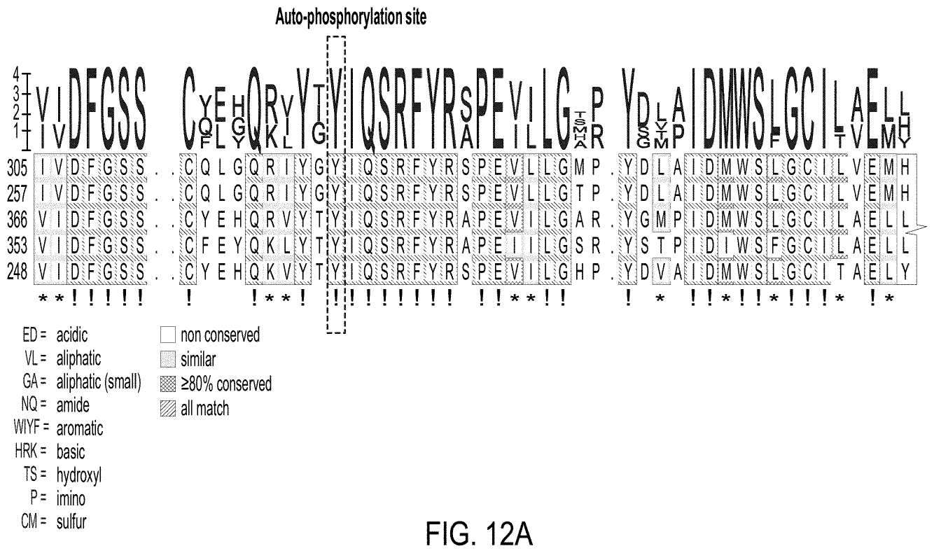

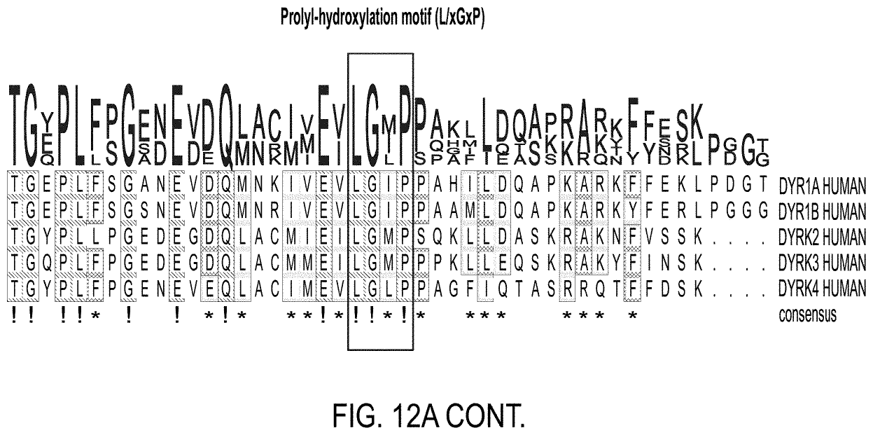

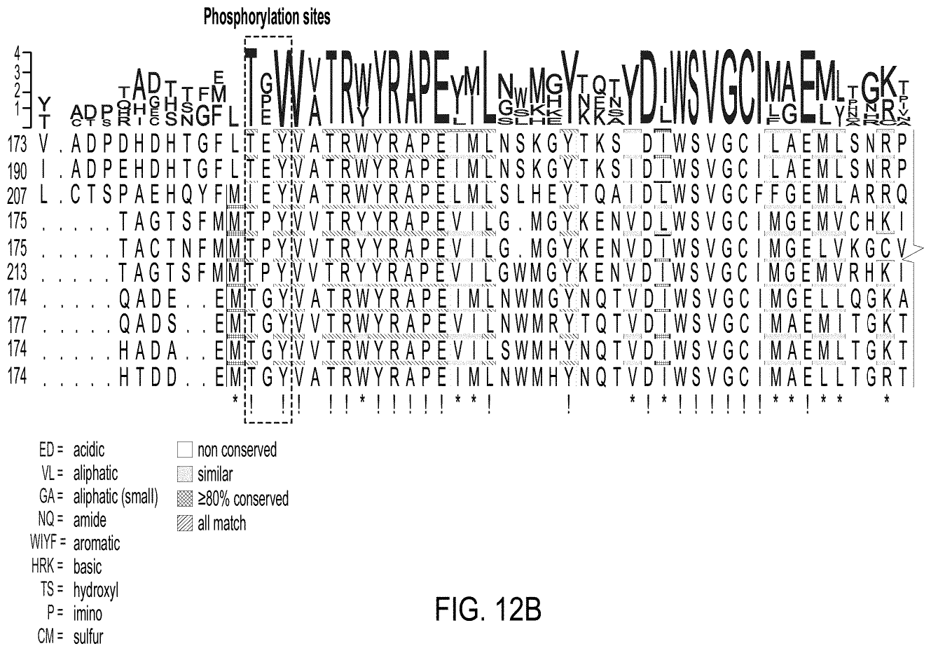

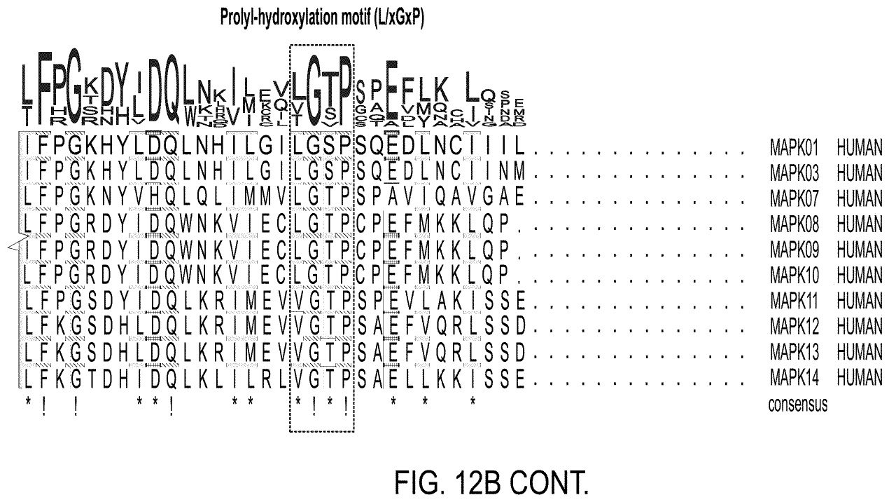

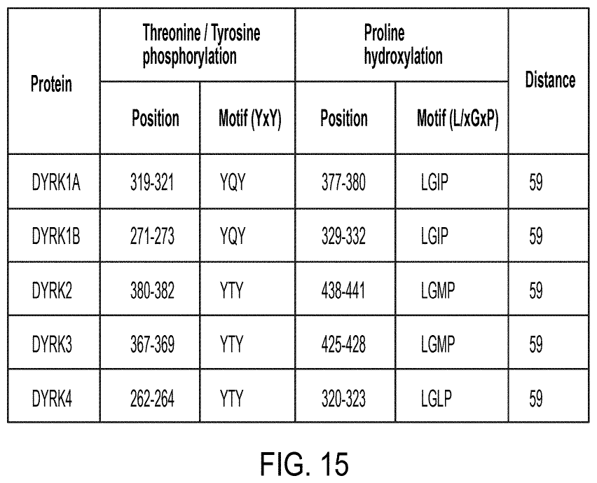

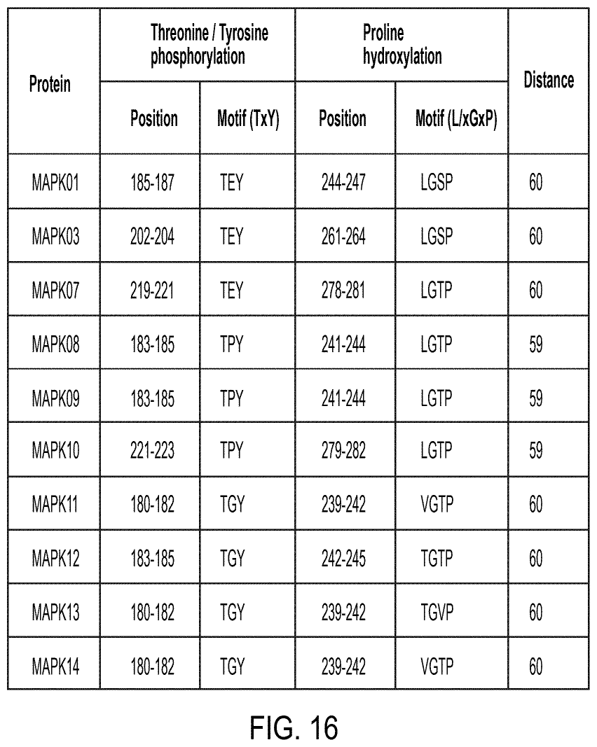

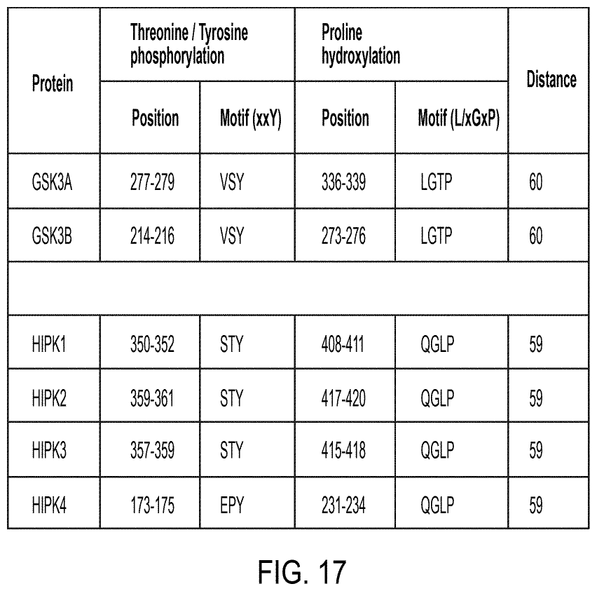

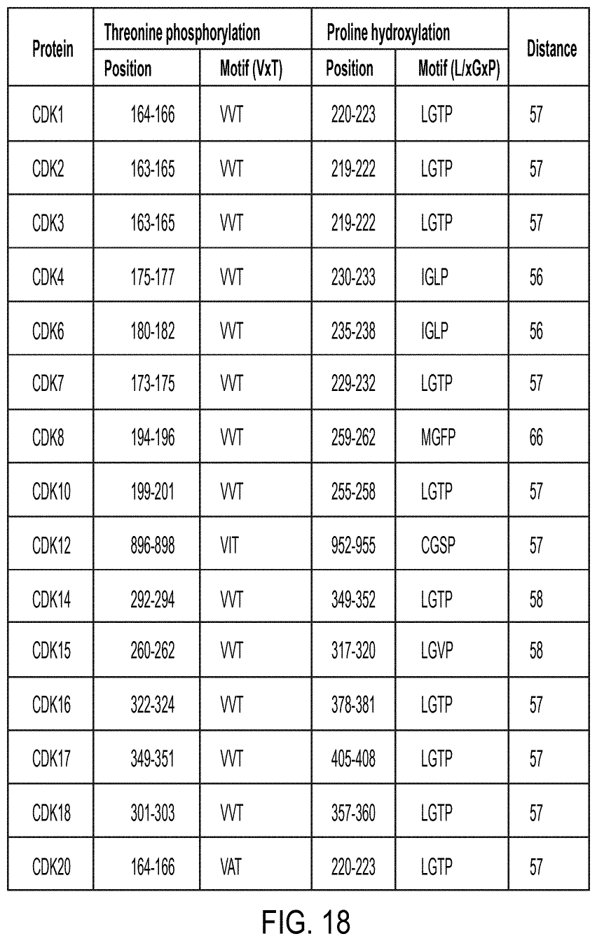

The hydroxylated proline of DYRK1A and DYRK1B is in a highly conserved L/xGxP motif present in most of the kinases composing the CMGC family (DYRK, MAPK, GSK3, HIPK and CDK, FIGS. 12, 13 and FIGS. 15-18). The motif lies just after the αG helix at the N-terminal end of the CMGC/MAPK insert, a distinctive segment of the CMGC group in the C-terminal lobe (Kannan and Neuwald, 2004) (FIG. 6A). This conserved proline residue in p38α (MAPK14, P242 corresponding to the hydroxylated DYRK1B P332) is a key pivot point in its 3D structure, allowing rotation of a cap structure that regulates access to a cavernous hydrophobic pocket (Perry et al., 2009). Previous studies have identified the pocket as a non-ATP-binding allosteric site controlling phosphorylation and kinase activity of CMGC kinases (Astolfi et al., 2018; Yueh et al., 2019). Consistent with this notion, several molecules, including non-ATP-site binders, regulate the activity of CMGC kinases through high-affinity interactions with this pocket (Astolfi et al., 2018; Comess et al., 2011; Diskin et al., 2008; Perry et al., 2009). The 4-position in P242, which is the atomic target of hydroxylation, was observed to be in direct contact with an octyl-glucoside ligand in the high-resolution crystallographic structure of p38α (FIG. 6A, inset magnification) (Comess et al., 2011; Diskin et al., 2008). To investigate whether the hydroxylation of the conserved proline might impact allosteric ligands that regulate autophosphorylation and activation of CMGC kinases in the 3D structure, the effect of P242 hydroxylation was modeled in the crystallographic p38α structure on its octyl-glucoside lipid allosteric activator. An enormous increase in van der Waals energy was calculated with 4-hydroxyl-P242, indicative of a clash with the ligand (FIG. 6B). Similarly, a 3D homology model was built of DYRK1B based on the same liganded crystallographic p38α structure (FIG. 6C) Despite the low sequence identity between DYRK1B and p38α, energy minimization of the octyl-glucoside ligand along with its surrounding shell of DYRK1B amino acid side chains resulted in a low energy, unclashed conformation similar to that seen in p38α. The model suggests the presence of an equivalent ligand binding pocket in DYRK1B (FIG. 6D). Thus, the 3D structure is consistent with the findings, showing that proline hydroxylation directly clashes structurally with a CMGC-specific, allosteric ligand in these kinases. It also suggests that endogenous allosteric repressor molecules may operate to negatively regulate the cellular activity of at least some CMGC kinases and such negative regulator(s) may be displaced upon proline hydroxylation.

To experimentally ask whether P242 of p38α is hydroxylated and determine the consequences of mutation of P242 for p38α activation, FLAG-p38α wild type and the P242A mutant protein were expressed. Wild type but not p38α-P242A was hydroxylated on proline (FIG. 6E). Western blot analysis of wild type and P242A p38α with an antibody that detects the T180-Y182 phosphorylated and active form of p38α showed that the P242A mutation reduced p38α activation (FIG. 6F).

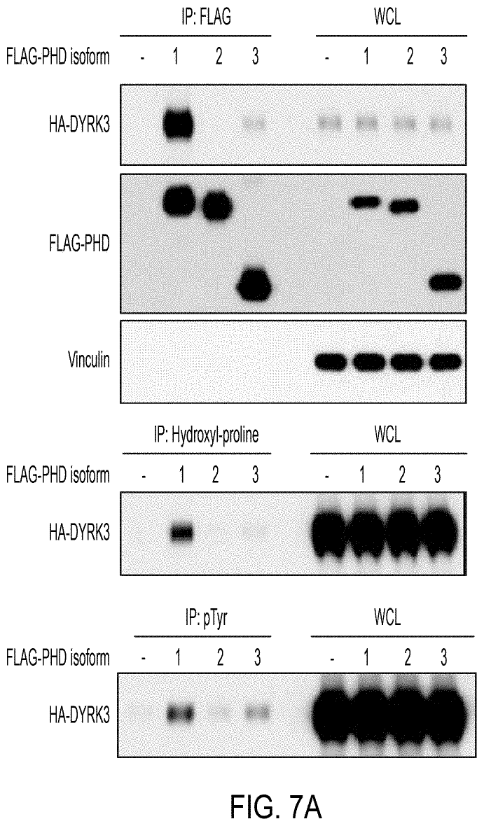

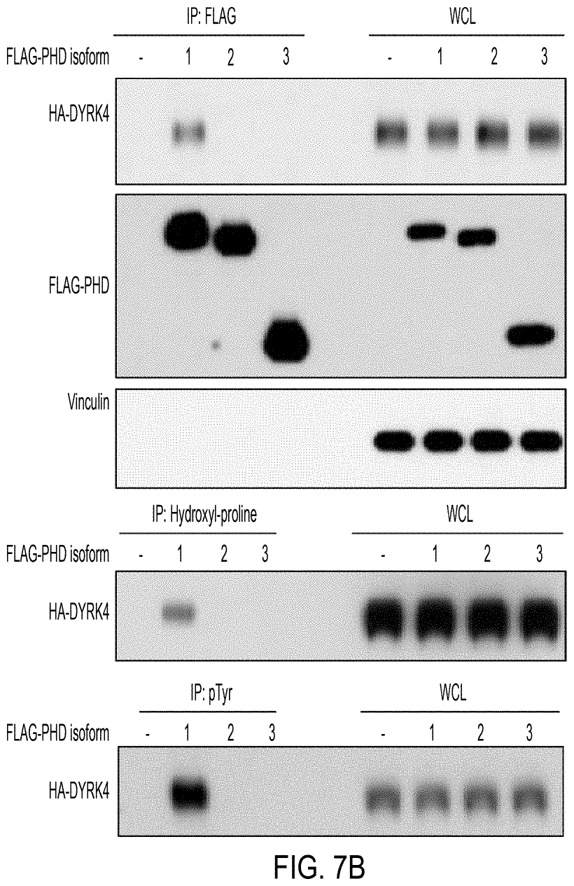

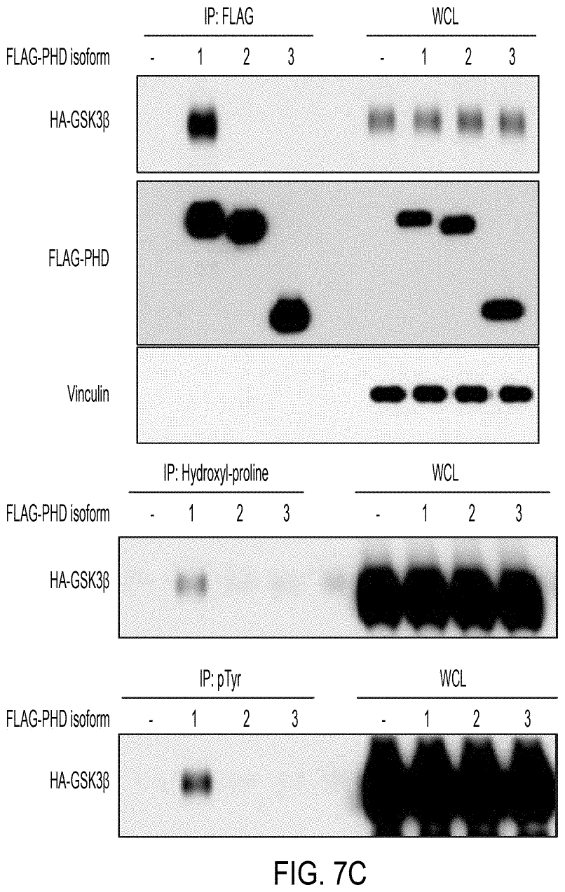

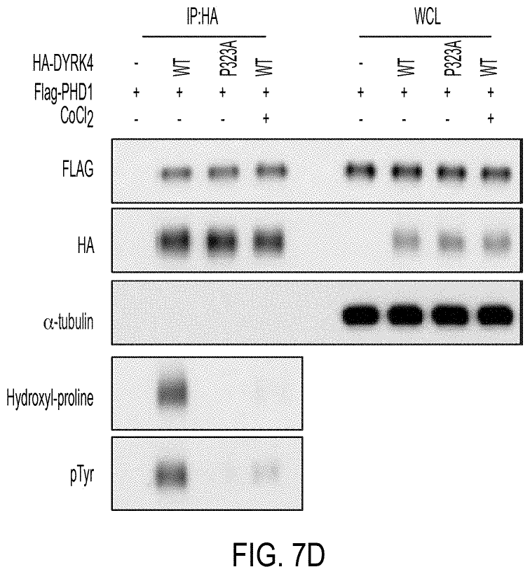

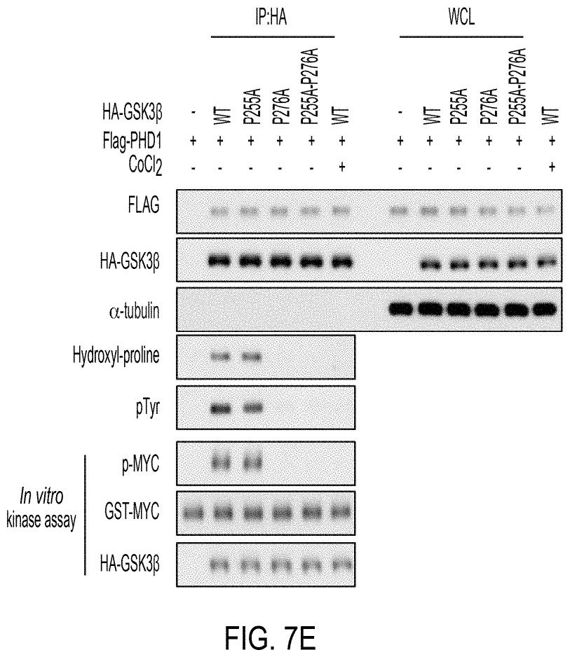

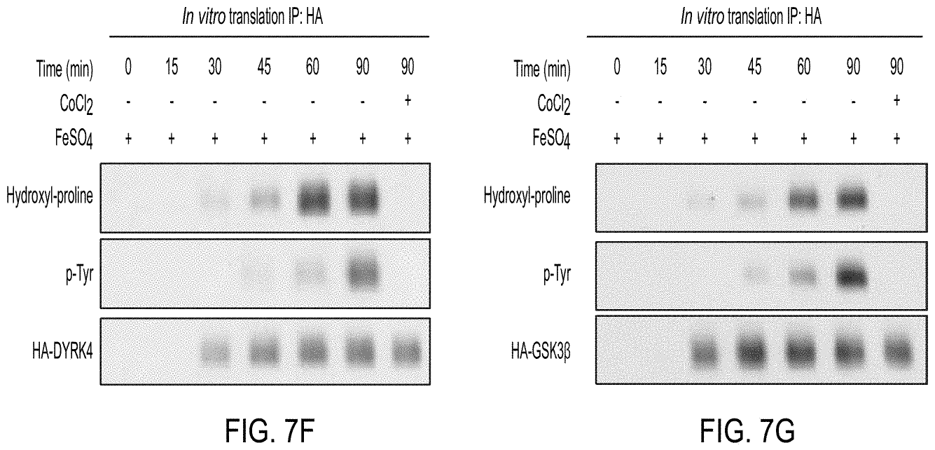

Next, it was tested experimentally whether hydroxylation of the conserved proline at the CMGC insert is a general mechanism for activation of CMGC kinases. First, it was asked whether other DYRK proteins and GSK3 kinases are also substrates of PHD enzymes. We focused on DYRK3, DYRK4 and GSK3β. It was found that each of the three kinases bound efficiently to PHD1, minimally to PHD3 but not to PHD2 (FIGS. 7A-C). Consistently, PHD1 was the only hydroxylase that, when expressed in U87 cells, elicited significant prolyl hydroxylation of DYRK3, DYRK4 and GSK3β. Consistent with the effects on proline hydroxylation phospho-tyrosine immunoprecipitation of DYRK3, DYRK4 and GSK3β demonstrated that PHD1, but not PHD2 or PHD3, enhanced tyrosine autophosphorylation of the tested kinases (FIGS. 7A-C).

To determine whether the proline embedded in the L/xGxP consensus sequences of the kinase domains is the target of hydroxylation by PHD1, the corresponding proline was mutated to alanine in DYRK4 (P323) and GSK3β (P255 and P276 are included in the L/xGxP candidate consensus LGQP and LGTP, respectively) and it was asked whether these mutations altered proline hydroxylation and/or tyrosine autophosphorylation. As the c-MYC oncoprotein is a prominent substrate for the tumor suppressor activity of the GSK3β kinase (Welcker et al., 2004), the ability of wild-type, GSK3β-P255A and GSK3β-P276A to phosphorylate purified GST-c-MYC in in vitro was interrogated. The results of these experiments showed that DYRK4-P323A and GSK3β-P276A could not be hydroxylated by PHD1 and lost tyrosine autophosphorylation whereas GSK3β-P255A behaved as GSK3β wild type. Accordingly, in the presence of PHD1, GSK3β wild type and GSK3β-P255A but not GSK3β-P276A phosphorylated GST-c-MYC in an in vitro kinase assay. Consistent with the oxygen-dependency of PHD hydroxylases, treatment of PHD1-expressing cells with the hypoxia-mimicking agent CoCl₂ caused loss of prolyl hydroxylation and tyrosine autophosphorylation of DYRK4 and GSK3β (FIGS. 7D, E). CoCl₂ also abolished GSK3β-mediated phosphorylation of GST-c-MYC in the in vitro kinase assay (FIG. 7E).

Finally, the temporal relationship between proline hydroxylation and tyrosine autophosphorylation of DYRK4 and GSK3β was examined using in vitro rabbit reticulocyte transcription-translation. The time-course translation assay revealed that proline hydroxylation of either kinase preceded tyrosine autophosphorylation and CoCl₂ abolished proline hydroxylation and consequent tyrosine autophosphorylation (FIGS. 7F, G). Hence, proline hydroxylation by PHD1 is required for the activation of CMGC kinases.

### Discussion

Recently it was reported that DYRK1A and DYRK1B undergo prolyl hydroxylation by the PHD1 oxygen-sensitive prolyl hydroxylase. Prolyl hydroxylation resulted in the activation of DYRK1 kinases and initiated a tumor suppressive cascade whereby the ID2 protein, directly phosphorylated by DYRK1, lost the ability to bind and disrupt the ubiquitin ligase activity of VHL towards HIFα substrates (Lee et al., 2016).

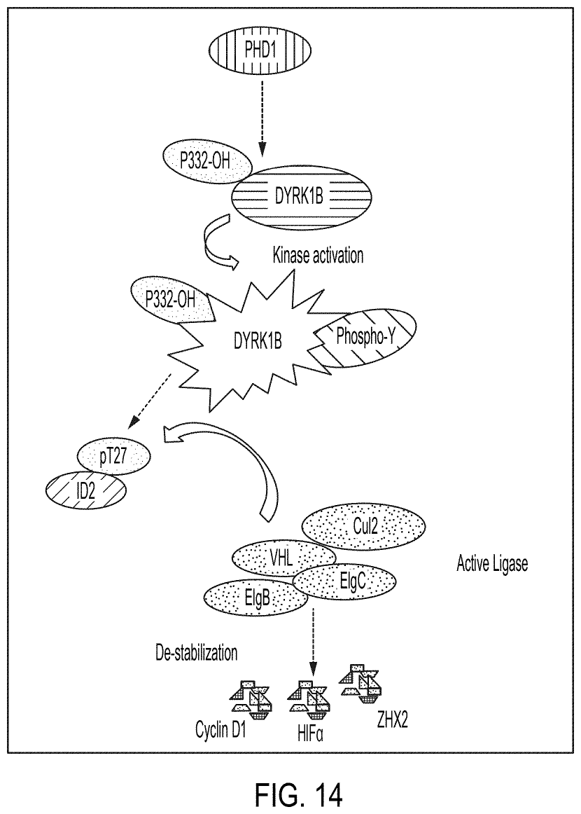

Here, a single, highly conserved proline was identified in the kinase domain of DYRK1A (P380) and DYRK1B (P332) as the acceptor residue for hydroxylation by PHD1. Recent reports suggested that bona-fide PHD substrates should be validated from in vitro prolyl hydroxylation assays in the presence of defined components (Cockman et al., 2019; Lee, 2019). Validated systems were used to confirm that DYRK1 proteins are prolyl hydroxylated (FIG. 1A, FIGS. 8B, C). Furthermore, DYRK1 emerged from an unbiased search of substrate-trapped interactors of PHDs in a quantitative proteomic screen (Rodriguez et al., 2016). Beside regulating ubiquitylation and destruction of the HIFα proteins, activation of DYRK1 kinase by prolyl hydroxylation enabled VBC^{CRL} ubiquitin ligase activity towards multiple oncoprotein substrates. Thus, proline hydroxylation is essential for DYRK1 kinase activity and the molecular events downstream of DYRK1 that are required for tumor suppression in vivo (FIG. 14).

The most unexpected finding of this work is that, rather than a specific mechanism for activation of DYRK1, prolyl hydroxylation by PHDs is likely an essential mechanism for the regulation of many eukaryotic protein kinases. In particular, the conservation of the L/xGxP motif in the CMGC group of protein kinases is suggestive of the critical function of this proline for the catalytic activity of CMGC kinases. This finding was experimentally validated for DYRK3, DYRK4, GSK3β and p38α. It was found that prolyl hydroxylation occurs either during translation and/or soon after protein synthesis and enables tyrosine phosphorylation and maturation through the HSP90 chaperone complex, ultimately promoting the catalytically active conformation.

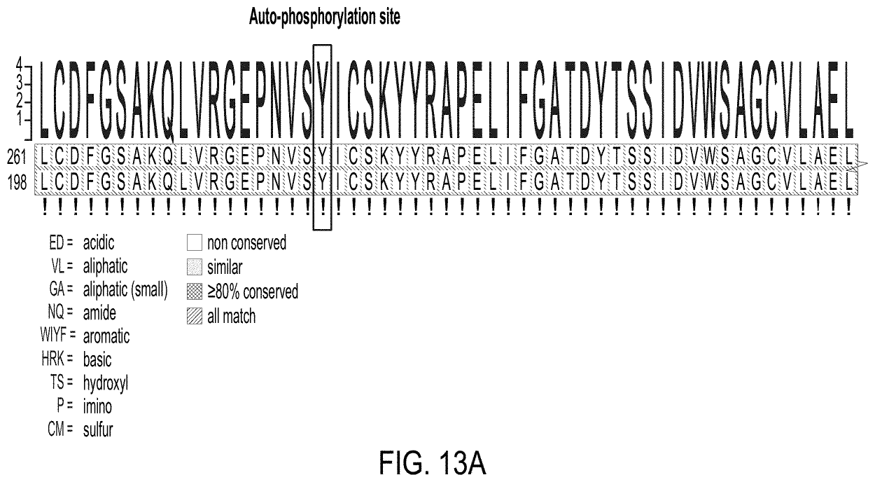

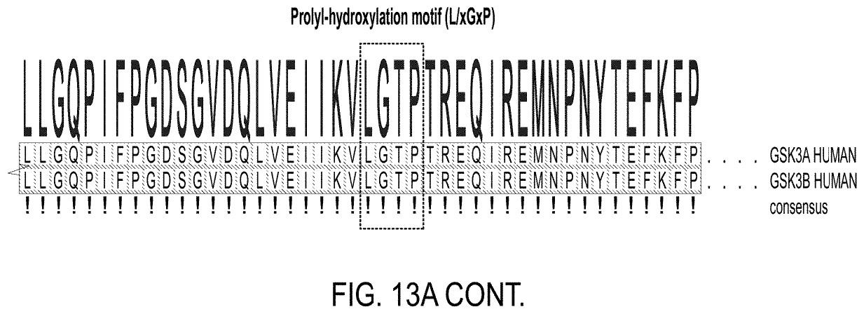

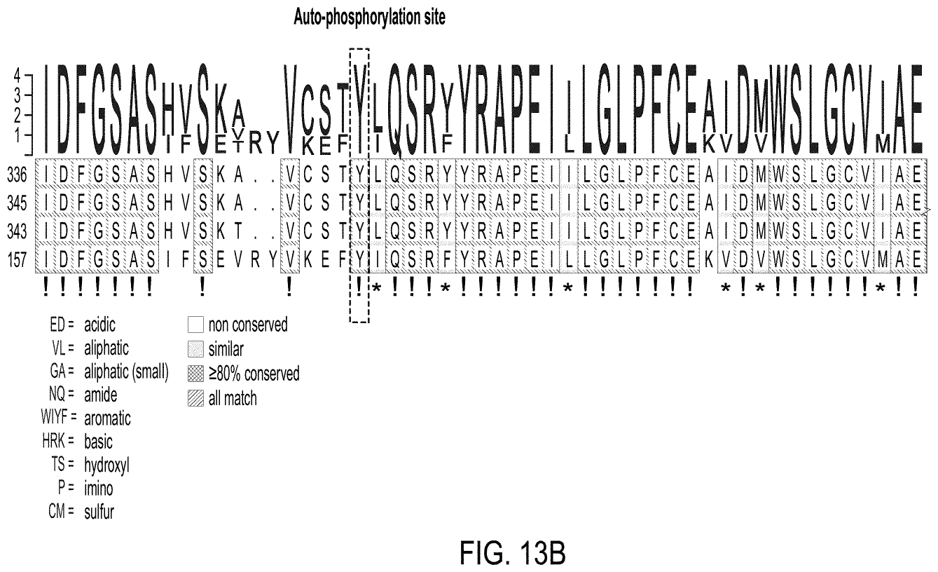

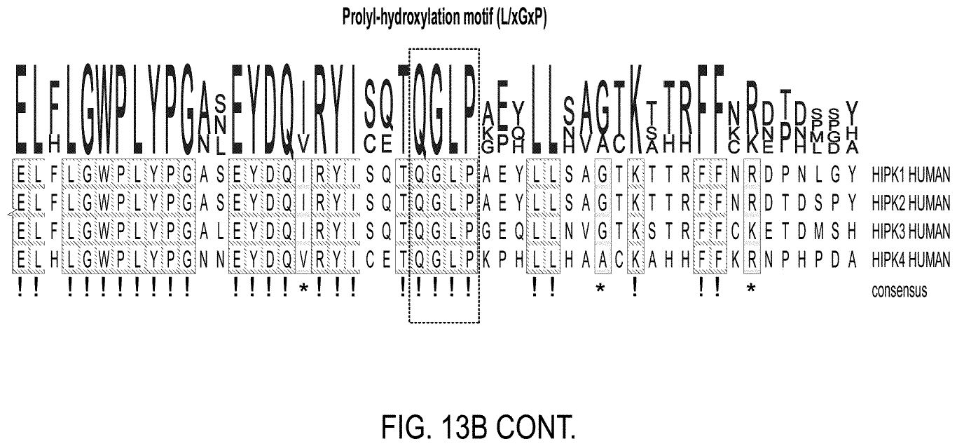

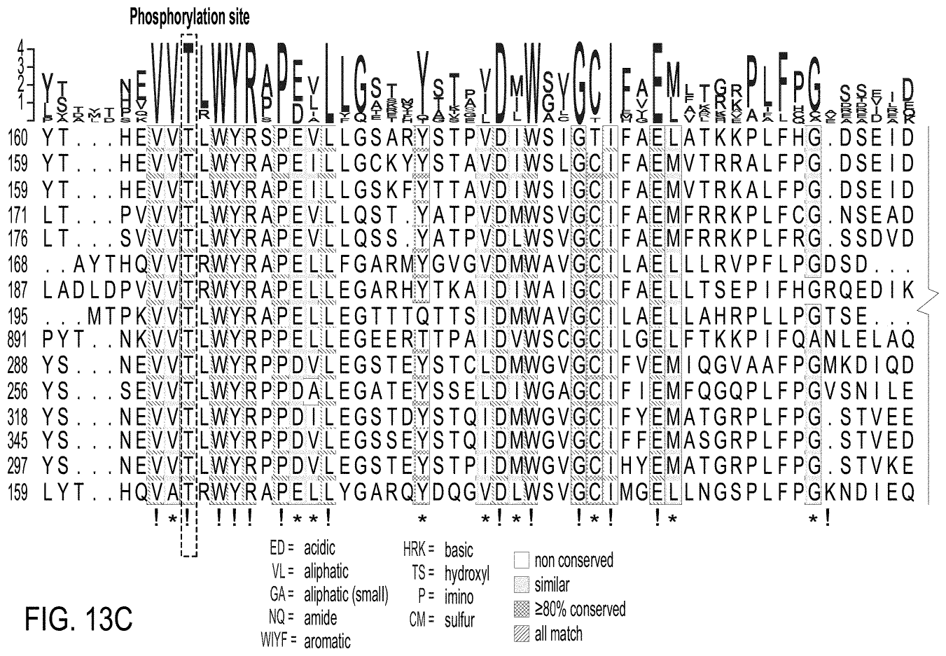

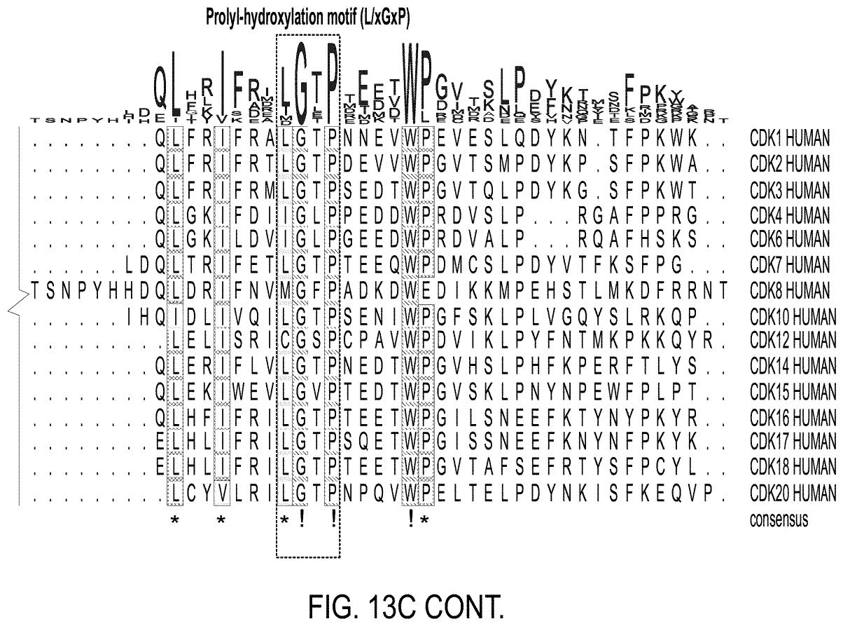

A critical regulatory step for the activation of protein kinases is transphosphorylation of the activation loop (Johnson et al., 1996). Recent studies have suggested that activation loop transphosphorylation is far more common in protein kinases than previously appreciated. However, it has remained uncertain how kinases catalyze this reaction when not in the active state (Beltrao et al., 2012; Nolen et al., 2004). It was postulated that a “prone-to-autophosphorylate” conformation is an intermediate step for kinases to catalyze the phosphotransfer reaction on their own activation-loop sites and other mechanisms in trans might be involved in facilitating the transition (Beenstock et al., 2016). The work, which focused on the group of kinases that autophosphorylate in a dimerization-independent manner and acquire a tyrosine auto-phosphorylated conformation (DYRK and GSK3), provides a clue to the steps leading to autophosphorylation and activation of protein kinases. It was shown that during and/or soon after translation of DYRK and GSK3 kinases, an essential event required for kinase activity is hydroxylation of a proline residue in the kinase domain. The evidence for a hydroxylated intermediate of these kinases is further supported by the 3D structural location of the target proline buried deep within the kinase C-lobe and not readily accessible to enzymes in the mature kinase form without unfolding of the protein. The analysis with mutants of the proline hydroxyl-acceptor site shows that loss of prolyl hydroxylation by PHD enzymes severely impacts the ability of CMGC kinases to proceed through the “prone-to-autophosphorylate conformation” and ultimately results in kinases that cannot achieve catalytic activity. The analysis of the GSK3β mutants P276A and P255A is particularly informative as it suggests that the molecular distance between the activation loop phosphorylation site and the hydroxylated proline is not random in the kinase sequence. In fact, the hydroxylated proline in CMGC kinases is spaced approximately sixty amino acids from the activation loop phosphorylation site (FIGS. 12, 13), suggesting high conservation of the requirement for proline hydroxylation. Given the broad role that activation-loop transphosphorylation has for activation of protein kinases and the pervasiveness of the proline hydroxylation modification of mammalian proteins (Gorres and Raines, 2010), it is likely that the translational proline hydroxylation of CMGC kinases reported here is a more general mechanism required for activation of protein kinases.

### Experimental Models and Subject Details

### Mice

4-6 weeks old athymic nude (Nu/Nu, Charles River Laboratories). Mice were housed in pathogen-free animal facility. No statistical method was used to pre-determine sample size. No method of randomization was used to allocate animals to experimental groups but males and females were included in the different treatment groups at approximately 50% ratio to exclude gender effects. All animal procedures including husbandry routines were approved by the Institutional Animal Care and Use Committee (IACUC) of Columbia University. Prior to experiments, mice were not involved in any procedure. The investigators were not blinded during outcome assessment. In none of the experiments did tumors exceed the maximum volume allowed according to approved IACUC protocol, specifically 20 mm in the maximum diameter.

### Method Details

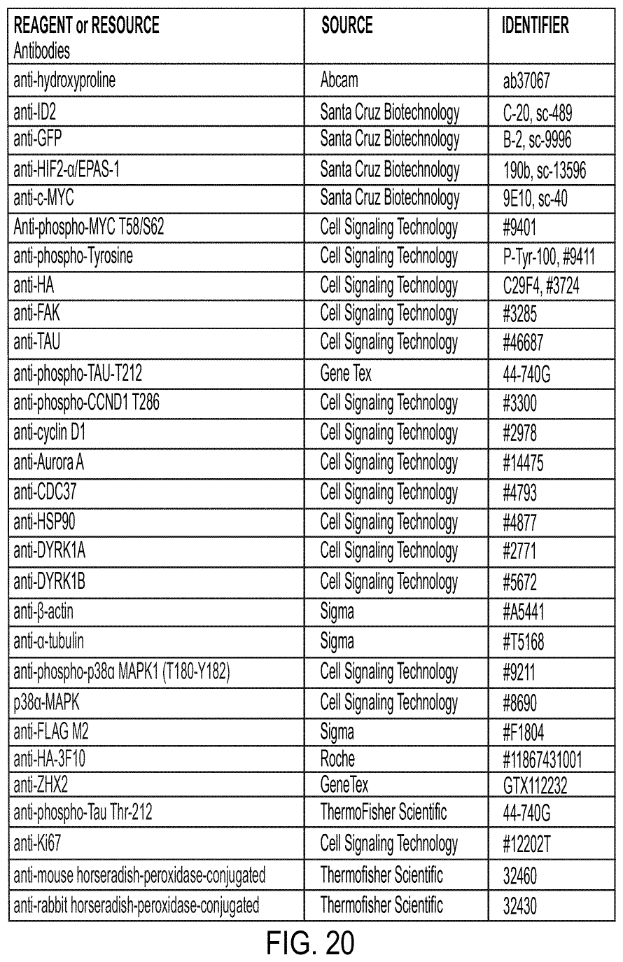

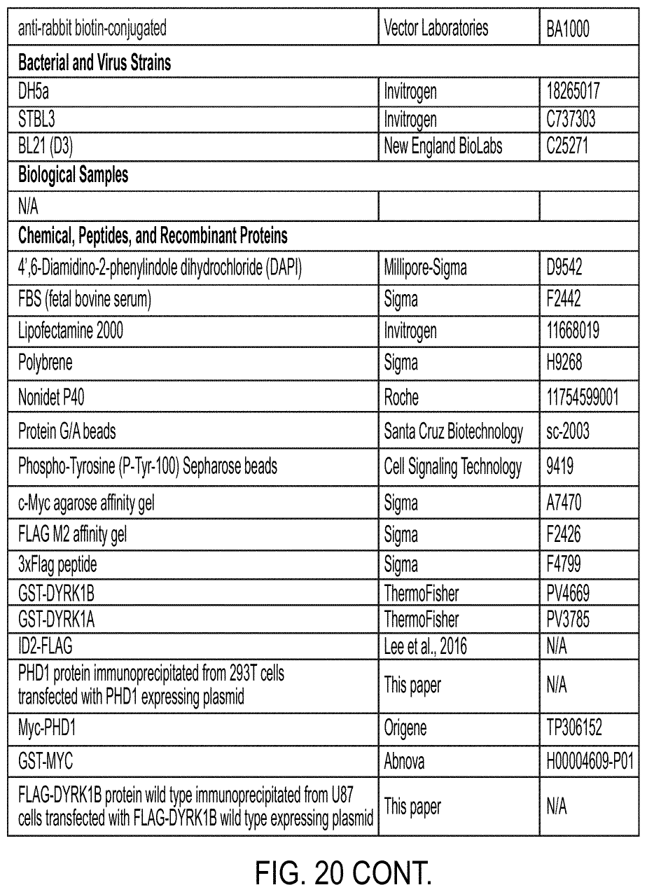

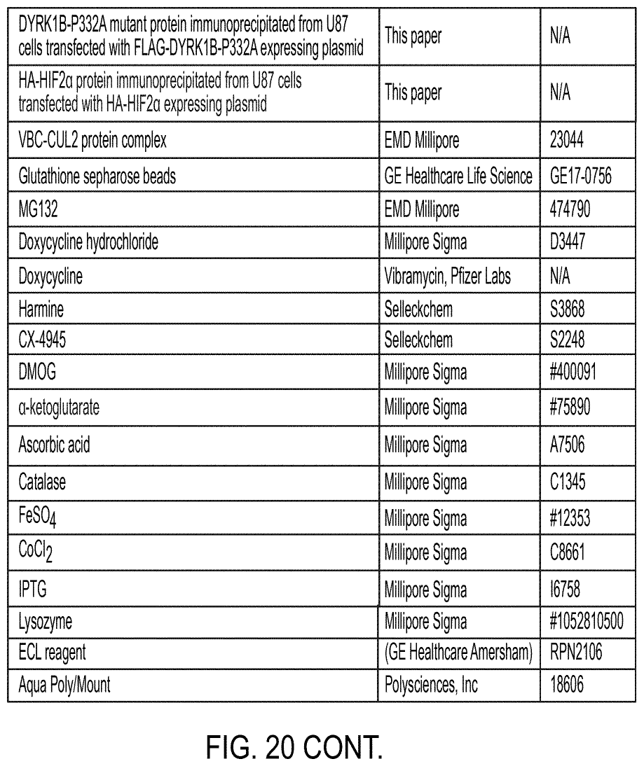

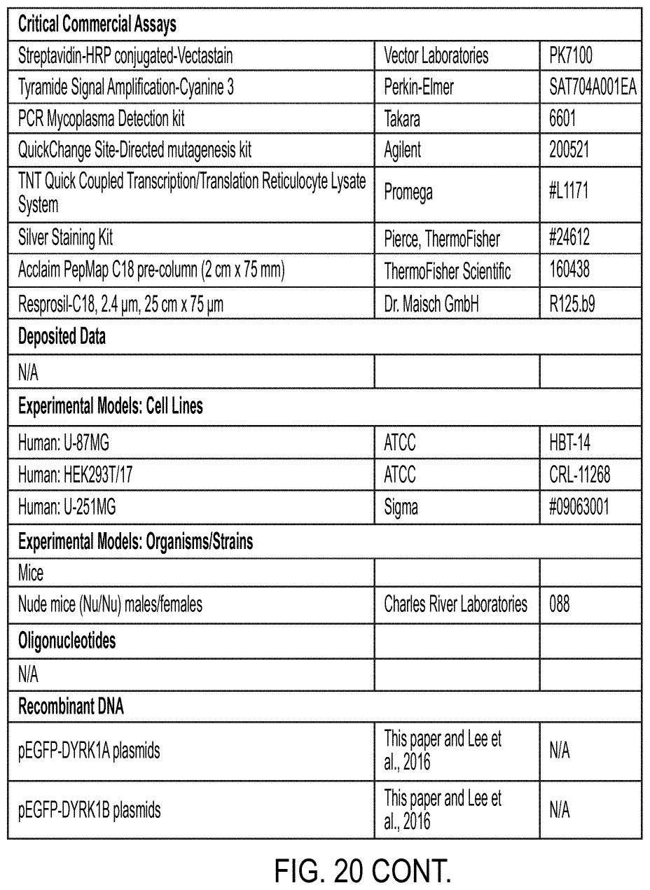

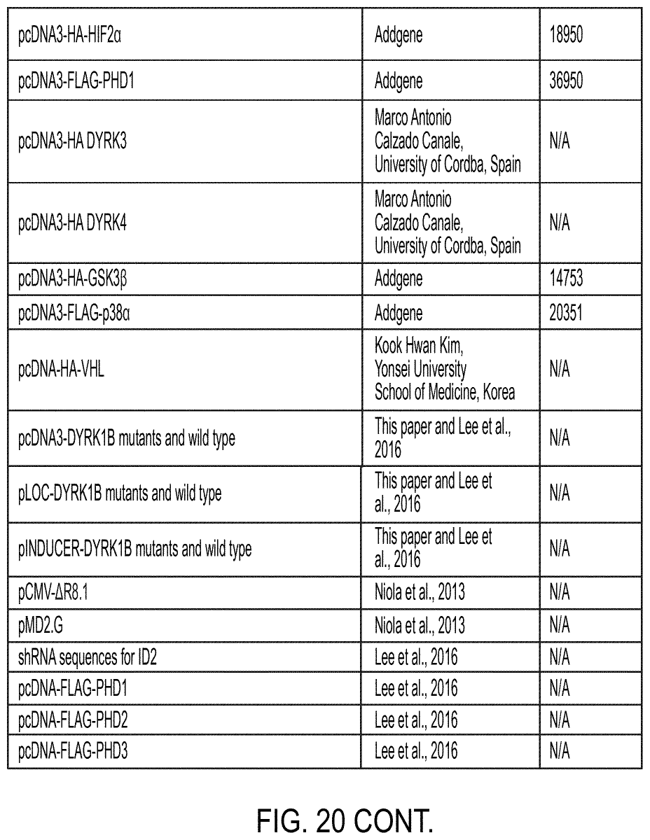

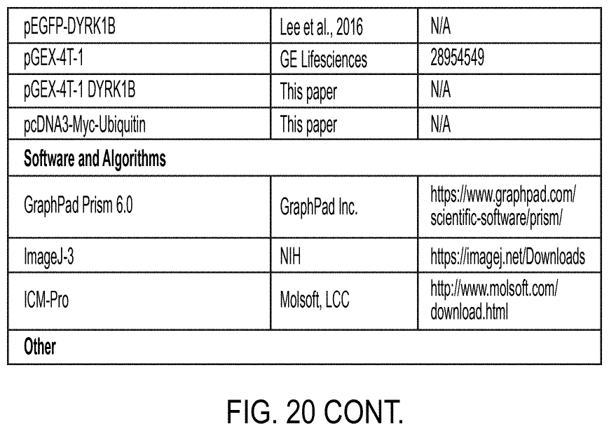

Reagents are resources are shown in FIG. 20.

### Plasmids, Cloning and Lentivirus Production

GFP-tagged DYRK1A or DYRK1B plasmids have been described previously (Lee 2016). pcDNA3-HA-DYRK3 and pcDNA3-HA-DYRK4 were kindly donated by Marco Antonio Calzato Canale (University of Cordoba, Spain). pcDNA3-HA-HIF2-α, pcDNA3-FLAG-PHD1, pcDNA3-HA-GSK3f3, and pcDNA3-FLAG-p38α were obtained from Addgene. pcDNA-HA-VHL was kindly provided by Kook Hwan Kim (Yonsei University School of Medicine, Korea). The cDNA for DYRK1B wild type and proline to alanine/glycine mutants were cloned into pcDNA3, pLOC and pINDUCER vectors. Proline to alanine/glycine mutants were generated by site-directed mutagenesis using the QuickChange Site-Directed Mutagenesis kit (Agilent) and resulting plasmids were verified by Sanger sequencing. FLAG, V5 or HA tags were added at the C-terminus. Lentiviral particles were obtained by co-transfection of lentiviral vectors with pCMV-ΔR8.1 and pMD2.G plasmids into HEK293T cells as previously described (Carro et al., 2010; Niola et al., 2013). shRNA sequences for ID2 have been previously published (Lee et al., 2016).

### Cell Culture and Treatment

U87 [ATCC HTB-14, (Allen et al., 2016)], and HEK293T (ATCC, CRL-11268) cell lines were acquired through American Type Culture Collection. U251 (Sigma, #09063001) cell line was obtained through Sigma. Cell lines were cultured in DMEM supplemented with 10% fetal bovine serum (FBS, Sigma). Cells were routinely tested for mycoplasma contamination using PCR Mycoplasma Detection Kit (Takara, #6601) and were found to be negative. Cells were transfected with Lipofectamine 2000 (Invitrogen) or calcium phosphate. To achieve expression of similar levels of wild type and mutant kinases, which were expressed at lower levels than the wild type, transfection was generally performed using plasmid DNA ratios of 1 wild type: 1.2-1.5 mutant (3.5 μg: 4.2-5 μg). Six μg of PHD enzyme plasmids were used in each transfection for 100 mm dish. For p38α analysis, cells were transfected with 1.5 μg and 2 μg of p38α wild type and p38α-P242A mutant, respectively. Lysates were prepared 20 hrs later in RIPA buffer (see section Immunoprecipitation and Immunoblot). Cells were transduced using lentiviral particles in medium containing 4 μg/ml of polybrene (Sigma, H9268). When indicated, cells were treated with the DYRK1 ATP competitive inhibitors harmine (10 μM, Selleckchem #S3868), CX-4945 (1 μM, Selleckchem #S2248), the hypoxia mimicking agents DMOG (1 mM, Millipore Sigma, #400091) or CoCl₂ (150 μM, Millipore Sigma, C8661) for 12 hours.

### Identification of Prolyl Hydroxylation Site of DYRK1B

To identify the sites of DYRK1B prolyl hydroxylation, U87 cells expressing FLAG tagged DYRK1B in the presence or the absence of PHD1 were used in immunoprecipitation assay performed with anti-hydroxyl-proline followed by anti-FLAG immunoprecipitation. Eluates containing DYRK1B protein were reduced with 5 mM DTT and alkylated with 10 mM iodoacetamide. Trypsin was added and samples were incubated at 37° C. for 16 hours. 10% of the digested samples were injected into the LC/MS system comprising the Dionex Ultimate 3000 RSLCnano system and Orbitrap Fusion (ThermoFisher Scientific). Samples were loaded onto the Acclaim PepMap C18 pre-column (2 cm×75 μm, ThermoFisher Scientific) with the loading pump at 3 μl/min for 3 min. A valve was switched to bring the pre-column into the flow path with the analytical column (Resprosil-C18, 2.4 μm, 25 cm×75 μm, Dr. Maisch GmbH) and a gradient from 2% buffer A (0.1% formic acid in water) to 20% buffer B (acetonitrile-0.1% formic acid) in 20 min to 30% buffer B in 2 min and 80% buffer B in 1 min at 200 nl/min. Data were acquired on an Orbitrap Fusion (ThermoFisher Scientific) which detects MS/MS of individual peptides. These included the oxidized and non-oxidized peptides as well as control peptides from DYRK1B.

### Immunoprecipitation and Immunoblot

Cells were lysed in NP40 lysis buffer [50 mM Tris-HCl, pH 7.5, 150 mM NaCl, 1 mM EDTA, 1% NP40, 1.5 mM Na₃VO₄, 50 mM sodium fluoride, 10 mM sodium pyrophosphate, 10 mM β-glycerolphosphate and EDTA free protease inhibitor cocktail (Millipore-Sigma #11836170001)] or RIPA buffer (50 mM Tris-HCl, pH 7.5, 150 mM NaCl, 1 mM EDTA, 1% NP40, 0.5% Sodium Deoxycholate, 0.1% Sodium dodecyl sulfate, 1.5 mM Na₃VO₄, 50 mM sodium fluoride, 10 mM sodium pyrophosphate, 10 mM (3-glycerolphosphate and EDTA free protease inhibitor cocktail). Lysates were cleared by centrifugation at 15,000 rpm for 15 min at 4° C. For immunoprecipitation, cell lysates were incubated with primary antibody (hydroxyl-proline, Abcam, ab37067) and protein G/A beads (Santa Cruz, sc-2003), phospho-tyrosine Sepharose beads (P-Tyr-100, Cell Signaling Technology #9419), c-Myc agarose affinity gel (Sigma, A7470), HA affinity agarose beads (Millipore-Sigma, #11815016001), and FLAG-M2 affinity beads (Sigma, F2426) at 4° C. overnight. Beads were washed with lysis buffer four times and eluted in 2×SDS sample buffer or FLAG peptide (Sigma, F4799). Protein samples were separated by SDS-PAGE and transferred to polyvinyl difluoride (PVDF) or nitrocellulose (NC) membrane. Membranes were blocked in Tris-buffered saline (TBS) with 5% non-fat milk and 0.1% Tween20, and probed with primary antibodies. Antibodies and working concentrations are: ID2 1:500 (C-20, sc-489), GFP 1:1000 (B-2, sc-9996), HIF2α/EPAS-1 1:250 (190b, sc-13596), c-MYC 1:1000 (9E10, SC-40) obtained from Santa Cruz Biotechnology; phospho-Tyrosine 1:1000 (P-Tyr-100, #9411), HA 1:1000 (C29F4, #3724 or #2367), FAK 1:1000 (#3285), TAU 1:1000 (#46687), phospho-CCND1 Thr-286 (#3300), cyclin D1 (#2978), AURKA 1:1000 (#14475), CDC37 1:1000 (#4793), HSP90 1:1000 (#4877), phospho-p38α MAPK 1 (T180-Y182):1000 (#9211), p38α-MAPK 1:1000 (#8690), DYRK1A 1:1000 (#2771), and DYRK1B 1:1000 (#5672), obtained from Cell Signaling Technology; β-actin 1:8000 (#A5441), α-tubulin 1:8000 (#T5168), and FLAG M2 1:500 (#F1804) obtained from Sigma; HA 1:1000 (3F10, #12158167001) obtained from Roche; ZHX2 1:1000 (GTX112232) obtained from GeneTex; phospho-Tau Thr-212 1:1000 (44-740G) obtained from ThermoFisher Scientific. Secondary antibodies anti-mouse, anti-rabbit and anti-rat horseradish-peroxidase-conjugated were purchased from Pierce and ECL reagent (GE Healthcare Amersham) was used for detection.

### Evaluation of Recombinant Proteins by Silver Staining

One-hundred ng of GST-DYRK1A and GST-DYRK1B proteins purified from baculovirus (ThermoFisher, GST-DYRK1B, PV4669 and GST-DYRK1A PV3785, respectively), 100 ng of MYC-PHD1 purified from HEK-293T cells (Origene, TP306152) and 150 ng of ID2-FLAG purified from E. coli (Lee et a., 2016) were resolved by SDS-PAGE. Gel was staining using Pierce Silver staining kit (ThermoFisher, #24612).

### In Vitro Hydroxylation Assay

For in vitro hydroxylation of DYRK1 kinases, 0.5 μg of substrate protein (purified from baculovirus, ThermoFisher, GST-DYRK1B, PV4669 or GST-DYRK1A PV3785) was incubated in 50 mM Tris-HCl, pH 7.5, 100 μM dithiothreitol (DTT), 1500 units/ml catalase, 100 μM FeSO₄, 1 mM ascorbic acid, 0.2 mM α-ketoglutarate, and 0.5 μg of PHD1 (Origene, TP306152) in 100 μL reaction volume. The reaction mixture was incubated for 30-60 min at 37° C. and then diluted by adding 400 μl NP40 buffer (50 mM Tris-HCl, pH 7.5, 150 mM NaCl, 1 mM EDTA, 1% NP40, 1.5 mM Na₃VO₄, 50 mM sodium fluoride, 10 mM sodium pyrophosphate, 10 mM β-glycerolphosphate and EDTA free protease inhibitor cocktail). GST-DYRK1 kinases were immunoprecipitated using the hydroxyl-proline antibody. Immunoprecipitated proteins were separated by SDS-PAGE and analyzed by western blot using DYRK1A or DYRK1B antibody.

### In Vivo and In Vitro Kinase Assays

In vivo kinase assay in glioma cells was performed using FLAG-DYRK1B, GFP-DYRK1B or HA-GSK3β exogenously expressed in HEK293T or U87 cells. Cell lysates were prepared in NP40 buffer (50 mM Tris-HCl, pH 7.5, 150 mM NaCl, 1 mM EDTA, 1% NP40, 1.5 mM Na₃VO₄, 50 mM sodium fluoride, 10 mM sodium pyrophosphate, 10 mM β-glycerolphosphate and EDTA free protease inhibitor cocktail). One mg of cellular lysates was immunoprecipitated overnight at 4° C. using GFP or FLAG antibodies (DYRK1B) or HA affinity matrix (GSK3β). The affinity matrix was washed with lysis buffer four times followed by two washes with kinase buffer [25 mM Tris-HCl, pH 7.5, 5 mM [β-glycerophosphate, 2 mM dithiothreitol (DTT), 0.1 mM Na₃VO₄, 10 mM MgCl₂, and 0.2 mM ATP] and incubated with 200 or 500 ng ID2-FLAG (purified from E. coli) or GST-MYC protein (Abnova, H00004609-P01) in kinase buffer for 30 min at 30° C. Kinase reactions were terminated by adding 2×SDS sample buffer, separated by SDS-PAGE and analyzed by western blot using phospho-T27-ID2 antibody or phospho-MYC antibody (Cell Signaling Technology, #9401).

For kinase assay using bacterially purified DYRK1B, GST-DYRK1B was cloned in pGEX-4T-1 plasmid. BL21(D3) E. coli were cultured for 3 hrs at 37° C. and protein expression was induced by adding 0.1 mM isopropyl-b-D-thiogalactoside (IPTG, Millipore-Sigma, 16758) for 3 hrs at 37° C. Bacterial pellet was lysed in PBS containing 5% glycerol, 1% NP40, and 100 μg/ml lysozyme (Millipore-Sigma #1052810500) and bound to glutathione-sepharose beads. In vitro hydroxylation was performed as described in the section “In vitro hydroxylation assay”. The affinity matrix was washed with NP40 buffer four times followed by two washes with kinase buffer (25 mM Tris-HCl, pH 7.5, 5 mM β-glycerophosphate, 2 mM dithiothreitol (DTT), 0.1 mM Na₃VO₄, 10 mM MgCl₂, and 0.2 mM ATP) and incubated with 500 ng ID2-FLAG (purified from E. coli) for 1 h at 30° C. Proteins were analyzed by western blot.

### Time Course of In Vitro Proline Hydroxylation-Tyrosine Phosphorylation of DYRK and GSK3β Kinases

In vitro transcription-translation of pcDNA3.1-FLAG-DYRK1B, HA-DYRK4 and HA-GSK3β (1 μg) was performed using the TNT Quick Coupled Transcription/Translation Reticulocyte Lysate System (Promega, #L1171) following the manufacturer's instructions in the presence of 100 μM FeSO₄, 300 μM CoCl₂ or 10 μM harmine as indicated. Reactions were terminated at the indicated times by the addition of 500 μl of ice cold NP40 lysis buffer [50 mM Tris-HCl, pH 7.5, 150 mM NaCl, 1 mM EDTA, 1% NP40, 1.5 mM Na₃VO₄, 50 mM sodium fluoride, 10 mM sodium pyrophosphate, 10 mM β-glycerophosphate and EDTA free protease inhibitor cocktail (Roche)] on ice. Samples were processed by immunoprecipitation using FLAG (DYRK1B) or HA (DYRK4 and GSK3β) affinity matrix. Immunoprecipitated proteins were separated by SDS-PAGE and analyzed by western blot using anti-hydroxyl-proline, anti-phospho-tyrosine antibody (47.5% of each reaction, respectively), and FLAG or HA antibody (5% of each reaction, total immunoprecipitated protein).

### In Vivo Analysis of Concurrent Proline Hydroxylation-Tyrosine Phosphorylation of DYRK1B

293T cells were transfected with FLAG-DYRK1B in the presence of PHD1 and 36 h later lysed in NP40 lysis buffer (50 mM Tris-HCl, pH 7.5, 150 mM NaCl, 1 mM EDTA, 1% NP40, 1.5 mM Na₃VO₄, 50 mM sodium fluoride, 10 mM sodium pyrophosphate, 10 mM β-glycerophosphate and EDTA free protease inhibitor cocktail). Cellular lysates were cleared by centrifugation at 15,000 rpm for 15 min at 4° C. Two mg of cell lysates were incubated with hydroxyl-proline antibody and protein G/A beads overnight at 4° C. After four washes, immunoprecipitated proteins were released from protein G/A beads in TBS containing 1% SDS and 2 mM EDTA at 100° C. for 5 minutes, diluted in 1% NP40 lysis buffer and re-precipitated using FLAG-M2 beads for 4 hrs at 4° C. Immunoprecipitated proteins were eluted with FLAG peptide and analyzed by western blot using phosphotyrosine (80% of the immunoprecipitation) and FLAG (5% of the immunoprecipitation) antibodies.

### Stoichiometry of Proline Hydroxylation of DYRK1B

The stoichiometry of DYRK1B proline hydroxylation was estimated by comparing the fraction of proline hydroxylation of DYRK1B and HIF2α, a well-established PHD substrate, using the hydroxyl-proline antibody in immunoprecipitation assay. Briefly, 293T cells were transfected with HA-DYRK1B or HA-HIF2-α in the presence of PHD1. Two mg of cellular lysates were prepared in NP40 lysis buffer (50 mM Tris-HCl, pH 7.5, 150 mM NaCl, 1 mM EDTA, 1% NP40, 1.5 mM Na₃VO₄, 50 mM sodium fluoride, 10 mM sodium pyrophosphate, 10 mM β-glycerophosphate and EDTA free protease inhibitor cocktail). Five-hundred μg of cleared lysates were immunoprecipitated with anti-hydroxyl-proline antibody (1:100). Immune complexes were collected with protein A/G agarose beads, washed 5 times in lysis buffer, and eluted in SDS loading buffer. Serial dilutions of total cellular lysates were loaded on SDS-PAGE together with 50% percent of the hydroxyl-proline immunoprecipitated HIF2-α and 100% of immunoprecipitated DYRK1B, respectively to obtain bands of similar intensities. Western blot was performed using HA antibody. The relative amount of proline hydroxylated species in DYRK1B and HIF2-α was determined by densitometry quantification of the immunoblots (hydroxyl-proline HIF2-α and DYRK1B immunoprecipitates and total cellular lysates) using the ImageJ software (NIH).

### GST-VHL Pull Down-HIF2α Capture Assay and In Vitro Binding Assay

Reagents were obtained as follow: 1) FLAG-DYRK1B wild type and FLAG-DYRK1B-P332A mutant were immunoprecipitated from U87 cells transfected with FLAG-DYRK1B wild type and FLAG-DYRK1B-P332A expressing plasmids and treated with vehicle or CoCl₂ 24-36 hrs after transfection. In preliminary experiments it was determined that complete elimination of proline hydroxylation on DYRK1 kinase was obtained by treating cells with 150 mM CoCl₂ for 16 hrs. Immunoprecipitated DYRK1B proteins were used to phosphorylate 500 ng of recombinant bacterially expressed ID2-FLAG in vitro as described in the section “In vitro kinase assay”; 2) HA-HIF2-α was produced in U87 cells transfected with the corresponding plasmid and treated with CoCl₂ or vehicle for 5 hours 24-36 hrs after transfection. In preliminary experiments it was determined that complete elimination of proline hydroxylation on HIF2-α was obtained by treating cells with 150 mM CoCl₂ for 5 hrs; 3) VBC protein complex including (GST-VHL, GST-ELO C, ELOB, Hist-CUL2 and RBX1) was purchased from Millipore Sigma (#23044M). The products of DYRK1B kinase reactions (phosphorylated/unphosphorylated T27-ID2) were used to test the binding between components of the VHL complex (VHL and CUL2) and the interaction between HIF2α (proline hydroxylated/un-hydroxylated) and VHL. Binding reactions included ID2-FLAG from in vitro DYRK1B kinase assay, 500 ng of VCB complex, and 200 μg of cell lysate containing HA-HIF2-α in binding buffer (50 mM Tris-Cl, pH 7.5, 100 mM NaCl, 1 mM EDTA, 10 mM β-glycerophosphate, 10 mM Sodium pyrophosphate, 50 mM sodium fluoride, 1.5 mM Na₃VO₄, 0.2% NP40, 10% glycerol, 0.1 mg/ml BSA and EDTA free protease inhibitor cocktail). Binding assay was performed at room temperature for 1 hour followed by 3 hours at 4° C. Protein complexes were pulled-down using glutathione sepharose beads (GE Healthcare Life Science), washed three times with buffer composed of 50 mM Tris-HCl, pH 7.5, 250 mM NaCl, 1 mM EDTA, 1% NP40, 1.5 mM Na₃VO₄, 50 mM sodium fluoride, 10 mM sodium pyrophosphate, 10 mM β-glycerophosphate and EDTA free protease inhibitor cocktail and additional five times with the same buffer containing 150 mM NaCl. GST-bound proteins were separated by SDS-PAGE and analyzed by western blot using the indicated antibodies.

For the in vitro binding of FLAG-ID2 after DYRKB1 kinase assay presented in FIG. 1F, the 200 ng of FLAG-ID2 substrate was immunoprecipitated using FLAG affinity matrix. After 2 washes in binding buffer, binding reactions were prepared including 500 ng of VBC complex. Binding conditions and washes were as described above. FLAG-ID2 immunoprecipitates were tested by western blot for the presence of bound VHL, phosphorylated ID2 (anti-pT27-ID2 antibody) and FLAG-ID2.

### Ubiquitylation Assay

U87 cells were transfected with pEGFP-DYRK1B wild type and mutants, pcDNA3-HA-VHL and pcDNA3-Myc-Ubiquitin. 36 hours after transfection, cells were treated with 10 μM MG132 (EMD Millipore) for 10 hours. After two washes with ice-cold PBS, cells were collected and lysates prepared in 100 μl of buffer containing 50 mM Tris-HCl pH 8.0, 150 mM NaCl (TBS) and 2% SDS and boiled at 100° C. for 10 min. Lysates were diluted with 900 μl of lysis buffer containing 1% NP40. Immunoprecipitation was performed using 1 mg of cellular lysates. Ubiquitinated proteins were immunoprecipitated using anti-MYC antibody (c-MYC-9E10, SC-40, Santa Cruz Biotechnology) and analyzed by western blot using the indicated antibodies.

### Sub-Cellular Fractionation

U87 cells were transfected with FLAG tagged DYRK1B wild type, DYRK1B-P332A, or DYRK1B-K140R. Cells were counted and 2×10⁶ cells from each transfection were harvested in ice cold PBS and resuspended in hypotonic buffer (10 mM Tris-HCl, pH 7.5, 10 mM KCl, 1.5 mM MgCl₂, 1 mM DTT, 0.1 mM EDTA, 0.05% NP40, 1.5 mM Na₃VO₄, 10 mM sodium fluoride, 10 mM sodium pyrophosphate and 10 mM β-glycerophosphate and EDTA free protease inhibitor cocktail). Cells were incubated on ice for 10 min and nuclei were collected by centrifugation at 1,200×g at 4° C. for 5 min. Nuclei were washed three times in hypotonic buffer, resuspended in 3 mM EDTA, 0.2 mM EGTA, 1 mM DTT, 1.5 mM Na₃VO₄, 10 mM sodium fluoride, 10 mM sodium pyrophosphate and 10 mM β-glycerophosphate and EDTA free protease inhibitor cocktail and incubated on ice for 30 min. The supernatant (nuclear soluble fraction) was collected by centrifugation at 1,700×g at 4° C. for 5 min. To prepare the cell insoluble fraction, the supernatant obtained after nuclear isolation was subjected to centrifugation to remove residual nuclei and debris and then the buffer components adjusted to a final concentration of 50 mM Tris-HCl, pH 7.5, 150 mM NaCl and 1% NP40 with protease inhibitors and incubated on ice for 30 min followed by centrifugation at 4° C., 15,000 rpm at 4° C. for 15 min. The supernatant from this step was the cytosolic fraction whereas the pellet was separated as NP40 insoluble fraction. NP40 insoluble fraction was washed in hypotonic buffer twice and resuspended in SDS sample buffer. Subcellular fractions represent equal cell numbers. Samples were separated by SDS-PAGE and analyzed by western blot using the indicated antibodies.

Structural Analysis and Modeling of the Hydroxylation Site in p38α and DYRK1B

A homology model of DYRK1B was built as previously described (Cardozo et al., 1995) based on the coordinates and sequence alignment with the crystallographic structure of DYRK1A (PDB ID 4MQL). PDB SMTX was used for analysis of the crystallographic structure of the kinase domain of p38α with relative locations of active site, octyl-glucoside ligand binding site, and CMGC insert. A tree description of a 3D model in internal coordinates was made from the SMTX coordinates as previously described (Maiorov and Abagyan, 1998) to model the energetic effect of P242 hydroxylation on bound octyl-glucoside. Van der Waals energy was calculated for the sphere of atoms 5 Å around P242 and octyl-glucoside for the model in which 4-hydroxylation of P242 was added and a wild-type model in which P242 was not hydroxylated. The electrostatic surfaces of the octylglucoside binding pocket in the p38α SMTX and DYRK1B/4MQL models were calculated and visualized as previously described (Abagyan and Totrov, 1994). All modeling and calculations were performed with ICM-Pro (Molsoft, LLC, La Jolla Calif.).

### Subcutaneous Xenograft Glioma Models

2×10⁵ U87 cells stably expressing a doxycycline inducible lentiviral vector coding for DYRK1B wild type, DYRK1B-P333A or DYRK1B-332A were injected subcutaneously in the right flank in 150 μl volume of saline solution. Mice carrying 150 mm³ subcutaneous tumors (approximately 21 days from injection) were treated with vehicle or doxycycline by oral gavage (Vibramycin, Pfizer Labs; 8 mg/ml, 0.2 ml/day). Tumor diameters were measured daily with caliper and tumor volumes estimated using the formula: width²×length/2=V (mm³). Mice were euthanized after 25 days of treatment or whenever the maximum tumor diameter reached 20 mm. Tumors were dissected and fixed in formalin for immunohistochemical analysis. Statistical significance of the difference in tumor volume during the entire experiment was determined by the analysis of covariance (ANCOVA) of the slopes calculated using GraphPad Prism 6.0 software package (GraphPad Inc.).

### Immunofluorescence of Cultured Cells and Primary Tissues

U87 glioma cells were plated on glass coverslips and transfected with pEGFP-DYRK1B wild type, pEGFP-DYRK1B-P333A or pEGFP-DYRK1B-P332A. Forty-eight hours later, cells were fixed with 4% paraformaldehyde; coverslips were mounted on glass slides using Aqua Poly/Mount (Polysciences, Inc.) Images were acquired under 40× magnification using an Olympus 1×70 microscope equipped with digital camera.

Tissue preparation and immunohistochemistry on mouse tissues were performed as previously described (Lee et al., 2016). Briefly, tumor sections were deparaffinized in xylene and rehydrated in a graded series of ethyl alcohol. Antigen retrieval was performed in citrate solution pH 6.0 using a decloaking chamber. After peroxidase block in 3% H₂O₂ for 15 min, slides were blocked overnight in 10% goat serum, 0.25% Triton X-100, lx PBS. Primary anti-Ki67 antibody (Cell Signaling #12202T, 1:1,000) was applied for 1 hour at room temperature. Sections were incubated in biotinylated secondary antibody for 1 hour, followed by 30 min of streptavidin-HRP conjugated (Vector Laboratories) and TSA-Cy3 (Perkin-Elmer). Nuclei were counterstained with DAPI (Sigma). Slides were mounted in Aqua Poly/Mount (Polysciences). Images were acquired under 20× magnification using an Olympus 1×70 microscope equipped with digital camera. Data are means±s.d. from 3 mice in each group. At least 300 cells were evaluated. Statistical significance was determined by the Student's t-test (two-tailed, unequal variance).

### Quantification and Statistical Analysis

Results in graphs are expressed as means±s.d. as indicated in figure legends, for the indicated number of observations. Statistical significance was determined by the Student's t-test (two-tailed, unequal variance) or ANCOVA using GraphPad Prism 6.0 software package (GraphPad Inc.). p-value<0.05 was considered significant and is indicated in figure legends.

### REFERENCES FOR EXAMPLE 1

Abagyan, R., and Totrov, M. (1994). Biased probability Monte Carlo conformational searches and electrostatic calculations for peptides and proteins. Journal of molecular biology 235, 983-1002.
Abu Jhaisha, S., Widowati, E. W., Kii, I., Sonamoto, R., Knapp, S., Papadopoulos, C., and Becker, W. (2017). DYRK1B mutations associated with metabolic syndrome impair the chaperone-dependent maturation of the kinase domain. Scientific reports 7, 6420.
Allen, M., Bjerke, M., Edlund, H., Nelander, S., and Westermark, B. (2016). Origin of the U87MG glioma cell line: Good news and bad news. Sci Transl Med 8, 354re353.
Aranda, S., Laguna, A., and de la Luna, S. (2011). DYRK family of protein kinases: evolutionary relationships, biochemical properties, and functional roles. FASEB J 25, 449-462.
Ashford, A. L., Dunkley, T. P., Cockerill, M., Rowlinson, R. A., Baak, L. M., Gallo, R., Balmanno, K., Goodwin, L. M., Ward, R. A., Lochhead, P. A., et al. (2016). Identification of DYRK1B as a substrate of ERK1/2 and characterisation of the kinase activity of DYRK1B mutants from cancer and metabolic syndrome. Cell Mol Life Sci 73, 883-900.
Ashford, A. L., Oxley, D., Kettle, J., Hudson, K., Guichard, S., Cook, S. J., and Lochhead, P. A. (2014). A novel DYRK1B inhibitor AZ191 demonstrates that DYRK1B acts independently of GSK3beta to phosphorylate cyclin D1 at Thr(286), not Thr(288). Biochem J 457, 43-56.
Astolfi, A., Manfroni, G., Cecchetti, V., and Barreca, M. L. (2018). A Comprehensive Structural Overview of p38alpha Mitogen-Activated Protein Kinase in Complex with ATPSite and Non-ATP-Site Binders. Chem Med Chem 13, 7-14.
Becker, W. (2012). Emerging role of DYRK family protein kinases as regulators of protein stability in cell cycle control. Cell Cycle 11, 3389-3394.
Beenstock, J., Mooshayef, N., and Engelberg, D. (2016). How Do Protein Kinases Take a Selfie (Autophosphorylate)? Trends Biochem Sci 41, 938-953.
Beltrao, P., Albanese, V., Kenner, L. R., Swaney, D. L., Burlingame, A., Villen, J., Lim, W. A., Fraser, J. S., Frydman, J., and Krogan, N. J. (2012). Systematic functional prioritization of protein posttranslational modifications. Cell 150, 413-425.
Bindra, R. S., Vasselli, J. R., Stearman, R., Linehan, W. M., and Klausner, R. D. (2002). VHL-mediated hypoxia regulation of cyclin D1 in renal carcinoma cells. Cancer Res 62, 3014-3019.
Cardozo, T., Totrov, M., and Abagyan, R. (1995). Homology modeling by the ICM method. Proteins 23, 403-414.
Carro, M. S., Lim, W. K., Alvarez, M. J., Bollo, R. J., Zhao, X., Snyder, E. Y., Sulman, E. P., Anne, S. L., Doetsch, F., Colman, H., et al. (2010). The transcriptional network for mesenchymal transformation of brain tumours. Nature 463, 318-325.
Cockman, M. E., Lippl, K., Tian, Y. M., Pegg, H. B., Figg, W. D. J., Abboud, M. I., Heilig, R., Fischer, R., Myllyharju, J., Schofield, C. J., et al. (2019). Lack of activity of recombinant HIF prolyl hydroxylases (PHDs) on reported non-HIF substrates. Elife 8.
Comess, K. M., Sun, C., Abad-Zapatero, C., Goedken, E. R., Gum, R. J., Borhani, D. W., Argiriadi, M., Groebe, D. R., Jia, Y., Clampit, J. E., et al. (2011). Discovery and characterization of non-ATP site inhibitors of the mitogen activated protein (MAP) kinases. ACS Chem Biol 6, 234-244.
Diskin, R., Engelberg, D., and Livnah, O. (2008). A novel lipid binding site formed by the MAP kinase insert in p38 alpha. J Mol Biol 375, 70-79.
Dowjat, W. K., Adayev, T., Kuchna, I., Nowicki, K., Palminiello, S., Hwang, Y. W., and Wegiel, J. (2007). Trisomy-driven overexpression of DYRK1A kinase in the brain of subjects with Down syndrome. Neurosci Lett 413, 77-81.
Gockler, N., Jofre, G., Papadopoulos, C., Soppa, U., Tejedor, F. J., and Becker, W. (2009). Harmine specifically inhibits protein kinase DYRK1A and interferes with neurite formation. FEBS J 276, 6324-6337.
Gorres, K. L., and Raines, R. T. (2010). Prolyl 4-hydroxylase. Crit Rev Biochem Mol Biol 45, 106-124.
Hammerle, B., Ulin, E., Guimera, J., Becker, W., Guillemot, F., and Tejedor, F. J. (2011). Transient expression of Mnb/Dyrk1a couples cell cycle exit and differentiation of neuronal precursors by inducing p27KIP1 expression and suppressing NOTCH signaling. Development 138, 2543-2554.
Hasanov, E., Chen, G., Chowdhury, P., Weldon, J., Ding, Z., Jonasch, E., Sen, S., Walker, C. L., and Dere, R. (2017). Ubiquitination and regulation of AURKA identifies a hypoxia-independent E3 ligase activity of VHL. Oncogene 36, 3450-3463.
Himpel, S., Tegge, W., Frank, R., Leder, S., Joost, H. G., and Becker, W. (2000). Specificity determinants of substrate recognition by the protein kinase DYRK1A. J Biol Chem 275, 2431-2438.
Jin, K., Lim, S., Mercer, S. E., and Friedman, E. (2005). The survival kinase Mirk/dyrk1B is activated through Rac1-MKK3 signaling. J Biol Chem 280, 42097-42105.
Johnson, L. N., Noble, M. E., and Owen, D. J. (1996). Active and inactive protein kinases:structural basis for regulation. Cell 85, 149-158.
Kaelin, W. G., Jr., and Ratcliffe, P. J. (2008). Oxygen sensing by metazoans: the central role of the HIF hydroxylase pathway. Mol Cell 30, 393-402.
Kalman, N., and Neuwald, A. F. (2004). Evolutionary constraints associated with functional specificity of the CMGC protein kinases MAPK, CDK, GSK, SRPK, DYRK, and CK2alpha. Protein Sci 13, 2059-2077.
Kentrup, H., Becker, W., Heukelbach, J., Wilmes, A., Schurmann, A., Huppertz, C., Kainulainen, H., and Joost, H. G. (1996). Dyrk, a dual specificity protein kinase with unique structural features whose activity is dependent on tyrosine residues between subdomains VII and VIII. J Biol Chem 271, 3488-3495.
Kershaw, N. J., and Babon, J. J. (2015). VHL: Cullin-g the hypoxic response. Structure 23, 435-436.
Kim, H., Lee, K. S., Kim, A. K., Choi, M., Choi, K., Kang, M., Chi, S. W., Lee, M. S., Lee, J. S., Lee, S. Y., et al. (2016). A chemical with proven clinical safety rescues Down syndrome-related phenotypes in through DYRK1A inhibition. Dis Model Mech 9, 839-848.
Lasorella, A., Benezra, R., and Iavarone, A. (2014). The ID proteins: master regulators of cancer stem cells and tumour aggressiveness. Nature reviews Cancer 14, 77-91.
Lee, F. S. (2019). Substrates of PHD. Cell Metab 30, 626-627.
Lee, S. B., Frattini, V., Bansal, M., Castano, A. M., Sherman, D., Hutchinson, K., Bruce, J. N., Califano, A., Liu, G., Cardozo, T., et al. (2016). An ID2-dependent mechanism for VHL inactivation in cancer. Nature 529, 172-177.
Litovchick, L., Florens, L. A., Swanson, S. K., Washburn, M. P., and DeCaprio, J. A. (2011). DYRK1A protein kinase promotes quiescence and senescence through DREAM complex assembly. Genes Dev 25, 801-813.
Lochhead, P. A., Sibbet, G., Morrice, N., and Cleghon, V. (2005). Activation-loop autophosphorylation is mediated by a novel transitional intermediate form of DYRKs. Cell 121, 925-936.
MacKenzie, E. D., Selak, M. A., Tennant, D. A., Payne, L. J., Crosby, S., Frederiksen, C. M., Watson, D. G., and Gottlieb, E. (2007). Cell-permeating alpha-ketoglutarate derivatives alleviate pseudohypoxia in succinate dehydrogenase-deficient cells. Mol Cell Biol 27, 3282-3289.
Maiorov, V., and Abagyan, R. (1998). Energy strain in three-dimensional protein structures. Fold Des 3, 259-269.
Manning, G., Whyte, D. B., Martinez, R., Hunter, T., and Sudarsanam, S. (2002). The protein kinase complement of the human genome. Science 298, 1912-1934.
Mazur-Kolecka, B., Golabek, A., Kida, E., Rabe, A., Hwang, Y. W., Adayev, T., Wegiel, J., Flory, M., Kaczmarski, W., Marchi, E., et al. (2012). Effect of DYRK1A activity inhibition on development of neuronal progenitors isolated from Ts65Dn mice. J Neurosci Res 90, 999-1010.
Mercer, S. E., Ewton, D. Z., Deng, X., Lim, S., Mazur, T. R., and Friedman, E. (2005). Mirk/Dyrk1B mediates survival during the differentiation of C2C12 myoblasts. J Biol Chem 280, 25788-25801.
Nguyen, H. C., Yang, H., Fribourgh, J. L., Wolfe, L. S., and Xiong, Y. (2015). Insights into Cullin-RING E3 ubiquitin ligase recruitment: structure of the VHL-EloBC-Cul2 complex. Structure 23, 441-449.
Niola, F., Zhao, X., Singh, D., Sullivan, R., Castano, A., Verrico, A., Zoppoli, P., Friedmann-Morvinski, D., Sulman, E., Barrett, L., et al. (2013). Mesenchymal high-grade glioma is maintained by the ID-RAP1 axis. J Clin Invest 123, 405-417.
Nolen, B., Taylor, S., and Ghosh, G. (2004). Regulation of protein kinases; controlling activity through activation segment conformation. Mol Cell 15, 661-675.
Park, J., Oh, Y., Yoo, L., Jung, M. S., Song, W. J., Lee, S. H., Seo, H., and Chung, K. C. (2010). Dyrk1A phosphorylates p53 and inhibits proliferation of embryonic neuronal cells. J Biol Chem 285, 31895-31906.
Perry, J. J., Harris, R. M., Moiani, D., Olson, A. J., and Tainer, J. A. (2009). p38alpha MAP kinase C-terminal domain binding pocket characterized by crystallographic and computational analyses. J Mol Biol 391, 1-11.
Rodriguez, J., Pilkington, R., Garcia Munoz, A., Nguyen, L. K., Rauch, N., Kennedy, S., Monsefi, N., Herrero, A., Taylor, C. T., and von Kriegsheim, A. (2016). Substrate-Trapped Interactors of PHD3 and FIH Cluster in Distinct Signaling Pathways. Cell Rep 14, 2745-2760.
Schofield, C. J., and Ratcliffe, P. J. (2004). Oxygen sensing by HIF hydroxylases. Nat Rev Mol Cell Biol 5, 343-354.
Selak, M. A., Armour, S. M., MacKenzie, E. D., Boulahbel, H., Watson, D. G., Mansfield, K. D., Pan, Y., Simon, M. C., Thompson, C. B., and Gottlieb, E. (2005). Succinate links TCA cycle dysfunction to oncogenesis by inhibiting HIF-alpha prolyl hydroxylase. Cancer Cell 7, 77-85.
Semenza, G. L. (2001). HIF-1, 0(2), and the 3 PHDs: how animal cells signal hypoxia to the nucleus. Cell 107, 1-3.
Sonamoto, R., Kii, I., Koike, Y., Sumida, Y., Kato-Sumida, T., Okuno, Y., Hosoya, T., and Hagiwara, M. (2015). Identification of a DYRK1A Inhibitor that Induces Degradation of the Target Kinase using Co-chaperone CDC37 fused with Luciferase nanoKAZ. Sci Rep 5, 12728.
Soundararajan, M., Roos, A. K., Savitsky, P., Filippakopoulos, P., Kettenbach, A. N., Olsen, J. V., Gerber, S. A., Eswaran, J., Knapp, S., and Elkins, J. M. (2013). Structures of Down syndrome kinases, DYRKs, reveal mechanisms of kinase activation and substrate recognition. Structure 21, 986-996.
Taipale, M., Krykbaeva, I., Koeva, M., Kayatekin, C., Westover, K. D., Karras, G. I., and Lindquist, S. (2012). Quantitative analysis of HSP90-client interactions reveals principles of substrate recognition. Cell 150, 987-1001.
Welcker, M., Orian, A., Jin, J., Grim, J. E., Harper, J. W., Eisenman, R. N., and Clurman, B. E. (2004). The Fbw7 tumor suppressor regulates glycogen synthase kinase 3 phosphorylation-dependent c-Myc protein degradation. Proc Natl Acad Sci USA 101, 9085-9090.
Yabut, O., Domogauer, J., and D'Arcangelo, G. (2010). Dyrk1A overexpression inhibits proliferation and induces premature neuronal differentiation of neural progenitor cells. J Neurosci 30, 4004-4014.
Yin, X., Jin, N., Shi, J., Zhang, Y., Wu, Y., Gong, C. X., Iqbal, K., and Liu, F. (2017). Dyrk1A overexpression leads to increase of 3R-tau expression and cognitive deficits in Ts65Dn Down syndrome mice. Sci Rep 7, 619.
Yueh, C., Rettenmaier, J., Xia, B., Hall, D. R., Alekseenko, A., Porter, K. A., Barkovich, K., Keseru, G., Whiny, A., Wells, J. A., et al. (2019). Kinase Atlas: Druggability Analysis of Potential Allosteric Sites in Kinases. J Med Chem 62, 6512-6524.
Zhang, J., Wu, T., Simon, J., Takada, M., Saito, R., Fan, C., Liu, X. D., Jonasch, E., Xie, L., Chen, X., et al. (2018). VHL substrate transcription factor ZHX2 as an oncogenic driver in clear cell renal cell carcinoma. Science 361, 290-295.
Zhang, J., and Zhang, Q. (2018). VHL and Hypoxia Signaling: Beyond HIF in Cancer. Biomedicines 6.
Zhang, Q., and Yang, H. (2012). The Roles of VHL-Dependent Ubiquitination in Signaling and Cancer. Front Oncol 2, 35.

What is claimed is:

1. A method of modulating kinase activity, the method comprising modulating the prolyl hydroxylation status of said kinase.

2. The method of claim 1, wherein the kinase is a CMGC kinase.

3. The method of claim 2, wherein the CMGC kinase is a dual specificity tyrosine-phosphorylation-regulated kinase (DYRK).

4. The method of claim 3, wherein the DYRK kinase is DYRK1A, DYRK1B, DYRK2, DYRK3, or DYRK4.

5. The method of claim 2, wherein the CMGC kinase is a MAPK kinase, a GSK3 kinase, a HIPK kinase, or a CDK kinase.

6. The method of claim 1, wherein the prolyl hydroxylation is achieved by a PHD hydroxylase.

7. The method of claim 6, wherein the PHD hydroxylase is PHD1.

8. The method of claim 1, wherein the prolyl hydroxylation is in a L/xGxP consensus sequence of the kinase.

9. The method of claim 4, wherein DYRK1A is hydroxylated on proline 380 of the amino acid sequence encoding DYRK1A.

10. The method of claim 4, wherein DYRK1B is hydroxylated on proline 332 of the amino acid sequence encoding DYRK1B.

11. The method of claim 1, wherein the modulation comprises increasing kinase activity.

12. The method of claim 1, wherein the modulation comprises decreasing kinase activity.

13. The method of claim 1, wherein prolyl hydroxylation of the kinase increases kinase activity.

14. The method of claim 1, wherein prolyl hydroxylation of the kinase suppresses tumor growth.

15. The method of claim 1, wherein prolyl hydroxylation of the kinase suppresses glioma growth.

16. The method of claim 1, wherein the modulator is a small molecule.

17. The method of claim 1, wherein the modulator acts on a PHD hydroxylase to alter its activity toward the kinase.

18. The method of claim 17, wherein the modulator is CoCl₂.

19. The method of claim 17, wherein the modulator is dimethyloxalylglycine (DMOG).

20. The method of claim 1, wherein the modulation comprises genetic engineering of the kinase or of a hydroxylase acting on the kinase.

21. A method of treating cancer in a subject in need thereof, the method comprising administering to the subject a therapeutically effective amount of a composition capable of modulating kinase activity.

22. The method of claim 21, wherein the kinase is a CMGC kinase.

23. The method of claim 22, wherein the CMGC kinase is a dual specificity tyrosine-phosphorylation-regulated kinase (DYRK).

24. The method of claim 23, wherein the DYRK kinase is DYRK1A, DYRK1B, DYRK2, DYRK3, or DYRK4.

25. The method of claim 22, wherein the CMGC kinase is a MAPK kinase, a GSK3 kinase, a HIPK kinase, or a CDK kinase.

26. The method of claim 21, wherein the composition promotes prolyl hydroxylation of said kinase.

27. The method of claim 26, wherein the prolyl hydroxylation is achieved by a PHD hydroxylase.

28. The method of claim 27, wherein the PHD hydroxylase is PHD1.

29. The method of claim 26, wherein the prolyl hydroxylation is in a L/xGxP consensus sequence of the kinase.

30. The method of claim 24, wherein DYRK1A is hydroxylated on proline 380 of the amino acid sequence encoding DYRK1A.

31. The method of claim 24, wherein DYRK1B is hydroxylated on proline 332 of the amino acid sequence encoding DYRK1B.

32. The method of claim 21, wherein the modulation comprises increasing kinase activity.

33. The method of claim 21, wherein the modulation comprises decreasing kinase activity.

34. The method of claim 26, wherein prolyl hydroxylation of the kinase increases kinase activity.

35. The method of claim 26, wherein prolyl hydroxylation of the kinase suppresses cancer growth.

36. The method of claim 35, wherein the cancer is a glioma, a breast cancer, a lung cancer, a bladder cancer, a colorectal cancer, melanoma, or a kidney cancer.

37. The method of claim 36, wherein the cancer is a glioma breast cancer.

38. The method of claim 21, wherein the modulator is a small molecule.

39. The method of claim 21, wherein the modulator acts on a PHD hydroxylase to alter the activity of the hydroxylase toward the kinase.

40. The method of claim 39, wherein the modulator is CoCl₂.

41. The method of claim 39, wherein the modulator is dimethyloxalylglycine (DMOG).

42. The method of claim 21, wherein the modulation comprises genetic engineering of the kinase or genetic engineering of a hydroxylase acting on the kinase.
